SEQUENCE LISTING (56 sequences)

SEQ ID NO: 1 - RNA, 85 nt, Homo sapiens
cgcuggcgacgggacauuauuacuuuugguacgcgcugugacacuucaaacucguaccgugaguaauaaugcgccguccacggca

SEQ ID NO: 2 - RNA, 22 nt, Homo sapiens
ucguaccgugaguaauaaugcg

SEQ ID NO: 3 - RNA, 22 nt, Artificial Sequence
  Synthetic ribonucleotide
cgcauuauuacucacgguacga

SEQ ID NO: 4 - RNA, 22 nt, Artificial Sequence
  Synthetic ribonucleotide
gcauuauuacucacgguacgag

SEQ ID NO: 5 - RNA, 22 nt, Artificial Sequence
  Synthetic ribonucleotide
gcgcauuauuacucacgguacg

SEQ ID NO: 6 - RNA, 24 nt, Artificial Sequence
  Synthetic ribonucleotide
gcgcauuauuacucacgguacgag

SEQ ID NO: 7 - RNA, 73 nt, Mus musculus
ugacagcacauuauuacuuuugguacgcgcugugacacuucaaacucguaccgugaguaauaaugcgcgguca

SEQ ID NO: 8 - RNA, 73 nt, Rattus norvegicus
ugacagcacauuauuacuuuugguacgcgcugugacacuucaaacucguaccgugaguaauaaugcgugguca

SEQ ID NO: 9 - RNA, 84 nt, Gallus gallus
gcuggugacggcccauuauuacuuuugguacgcgcugugacacuucaaacucguaccgugaguaauaaugcgcuguggucagca

SEQ ID NO: 10 - RNA, 85 nt, Macaca mulatta
cgcuggugaugggacauuauuacuuuugguacgcgcugugacacuucaaacucguaccgugaguaauaaugcgcuguccacagca

SEQ ID NO: 11 - RNA, 59 nt, Canis familiaris
cauuauuacuuuugguacgcgcugugacacuucaaacucguaccgugaguaauaaugcg

SEQ ID NO: 12 - DNA, 27 bp, Artificial Sequence
  Synthetic deoxyribonucleotide
tcacagcgcgtaccaaaagtaataatg

SEQ ID NO: 13 - DNA, 30 bp, Artificial Sequence
  Synthetic deoxyribonucleotide
gcgcattattactcacggtacgagtttgaa

SEQ ID NO: 14 - DNA, 28 bp, Artificial Sequence
  Synthetic deoxyribonucleotide
gtcacagcgcgtaccaaaagtaataatg

SEQ ID NO: 15 - DNA, 30 bp, Artificial Sequence
  Synthetic deoxyribonucleotide
tcacagcgcgtaccaaaagtaataatgtcc

SEQ ID NO: 16 - DNA, 27 bp, Artificial Sequence
  Synthetic deoxyribonucleotide
cattattactcacggtacgagtttgaa

SEQ ID NO: 17 - DNA, 29 bp, Artificial Sequence
  Synthetic deoxyribonucleotide
cgcattattactcacggtacgagtttgaa

SEQ ID NO: 18 - RNA, 101 nt, Danio rerio
gagccauuuuaacugcuucacaguccauuauuacuuuugguacgcgcuaggccagacucaaacucguaccgugaguaauaaugcacuguggcaguggguuu

SEQ ID NO: 19 - DNA, 31 bp, Artificial Sequence
  Synthetic deoxyribonucleotide
tgcattattactcacggtacgagtttgagtc

SEQ ID NO: 20 - DNA, 25 bp, Artificial Sequence
  Synthetic deoxyribonucleotide
gcctagcgcgtaccaaaagtaataa

SEQ ID NO: 21 - DNA, 25 bp, Artificial Sequence
  Synthetic deoxyribonucleotide
gcattattactcacggtacgagttt

SEQ ID NO: 22 - DNA, 25 bp, Artificial Sequence
  Synthetic deoxyribonucleotide
cctgaggaccagaaacagtctcacc

SEQ ID NO: 23 - DNA, 25 bp, Artificial Sequence
  Synthetic deoxyribonucleotide
gtctcctcgctcatctttccctcac

SEQ ID NO: 24 - RNA, 22 nt, Homo sapiens
uuuaacuaaauguaagguacga

SEQ ID NO: 25 - RNA, 20 nt, Homo sapiens
uuaccauauucaggguacga

SEQ ID NO: 26 - RNA, 23 nt, Homo sapiens
ggguccccacugccccgguacga

SEQ ID NO: 27 - RNA, 22 nt, Homo sapiens
ucuuuaaacgcucuagguacga

SEQ ID NO: 28 - RNA, 22 nt, Homo sapiens
aggcagguuuuguacgguacgu

SEQ ID NO: 29 - RNA, 23 nt, Homo sapiens
uguauaguacuggcaugguacgg

SEQ ID NO: 30 - DNA, 1335 bp, Homo sapiens
atgagcgaggagacggcgacttctgacaacgataatagttatgcacgagtgcgagctgtggtgatgacccgagatgactcaagtggtggatggttaccacttggagggagtggactaagcagcgtcactgtcttcaaagtccctcatcaggaagagaatggctgtgctgacttttttatccgtggagagcgactcagggacaaaatggtggttttggaatgtatgcttaaaaaagacctcatttataataaggtcactccaacatttcaccactggaagattgatgacaaaaagtttggtcttacgtttcaaagtcctgctgatgctagggcttttgatagaggtatccgaagagctatagaggatatttctcaaggatgccccgaatcaaaaaatgaagctgaaggggcagatgacttacaagcaaatgaagaggattcttccagttctctagtgaaggatcacctttttcagcaagagacagttgttaccagtgagccttatagaagctcaaatataagaccttctccctttgaagatctgaatgccagaagagtctacatgcaaagccaagccaatcagataacatttggtcagccaggcttggacattcagagcagaagtatggaatacgtacagcggcaaatatccaaggaatgtggaagcctaaagtcccaaaatagggtccctttgaaatcaatcagacatgtcagctttcaagatgaggatgagattgtcagaataaaccctcgagatatcttaatacgtcgctatgcagactacagacatcctgacatgtggaaaaatgacttggaaagagatgatgctgattccagtattcagttttctaaaccagacagtaaaaaatcagactatctgtactcttgtggggatgagactaagttaagttcacccaaagactctgtggtatttaagacgcagccttcctcattaaaaattaagaagtcaaaacgaagaaaagaggatggtgaacgttctcgctgcgtatactgccaggaaaggtttaatcatgaagaaaatgtcaggggaaaatgtcaggatgctccagaccctattaaaagatgcatatatcaagttagttgcatgctctgtgcagagagcatgttgtatcattgtatgtcagactcagagggagatttttctgatccctgttcgtgtgacactagcgacgacaagttctgcttgcgatggttagccctggtagctttgtctttcattgtaccatgtatgtgctgctacgtccctttgagaatgtgccatcgctgtggtgaggcatgtggttgctgtggtgggaaacataaagctgctggatga

SEQ ID NO: 31 - DNA, 2187 bp, Homo sapiens
atggcgggccctgagggcttccagtaccgcgctctgtacccgttccgccgggagcggccggaggacctggagctgctgcccggcgacgtgctggtagtgagccgggcggccttgcaggcgctgggcgtggccgagggtggcgagcgctgcccacagagcgtgggctggatgcccggcctcaacgagcgcacacggcagcgaggtgacttccctggcacctatgtggagttcctggggcccgtggccctggcccggcccggccctcgcccacggggcccccgcccactgcccgccaggccccgtgatggggcccctgagccaggcctcacactccccgacttgcccgagcagttctccccacctgatgtggctccccctcttctggtgaagcttgtggaggccattgaaaggacagggctggacagcgaatctcactaccgcccggagctgcccgcaccgcgtacagactggtccctgagcgacgtggatcagtgggacacggcagccctggctgacggcattaagagcttcctgctggcactgcccgcgccgctcgtgacccccgaggcctcggccgaggcgcgccgggccctgcgggaggccgcggggcccgtggggccggcgctggagccaccgacgctgccgctgcaccgcgcgctcacgctgcgcttcctgctccagcacctgggccgcgtggcccgccgcgccccggccctgggtcccgcggtccgggccctgggcgccacctttgggccgctgctgctgcgcgcgccgccgccgccgtcctcgccgccgccagggggcgctcccgacgggagtgagcccagccctgacttcccggcgctgctggtggagaagctgcttcaggaacacttggaagagcaggaggttgcgcccccagcgctgccgcctaaaccccccaaggcaaagccggcccccacagtcctggccaatggagggagcccaccctccctgcaggatgctgagtggtactggggggacatttcaagggaggaggtgaacgagaaactccgggacactcccgatggcaccttcctagtccgagatgcttctagcaagatccagggcgagtacacgctgaccctcaggaaaggcgggaacaataagctgatcaaggtcttccaccgagatgggcactatggcttctcagagccactcaccttctgctccgttgtggacctcatcaatcactaccgccacgagtctctggcccagtacaatgccaagctggacacacggctcctctaccctgtgtccaaataccagcaggaccagattgtcaaggaggacagcgtggaggcagtgggcgcccagcttaaggtctatcaccagcagtaccaggacaagagccgcgagtatgaccagctttatgaagagtacacacggacctcccaggagctgcagatgaagcgtactgcaattgaggccttcaatgagactatcaagatctttgaagagcagggccagactcaagagaaatgcagcaaggaatacctggagcgcttccggcgtgagggcaacgagaaagagatgcaaaggatcctgctgaactccgagcggctcaagtcccgcattgccgagatccatgagagccgcacgaagctggagcagcagctgcgggcccaggcctcggacaacagagagatcgacaagcgcatgaacagcctcaagccggacctcatgcagctgcgcaagatccgagaccagtacctcgtgtggctcacccagaaaggcgcccggcagaagaaaatcaacgagtggctggggattaaaaatgagactgaggaccagtacgcactcatggaggacgaggacgatctcccgcaccacgaggaacgcacttggtacgtgggcaagatcaaccgcacgcaggcagaggagatgctgagtggcaagcgggatggcaccttcctcatccgcgagagcagccagcggggctgctacgcctgctccgtggtagtggacggcgacaccaagcactgcgtcatctaccgcacggccaccggcttcggcttcgcggagccctacaacctgtacgggtcgctgaaggagctggtgctgcactaccagcacgcctcgctggtgcagcacaacgacgcgctcaccgtcaccctggcgcacccagtgcgcgccccgggccccggcccgccgcctgccgcccgctga

SEQ ID NO: 32 - DNA, 22 bp, Artificial Sequence
  Synthetic deoxyribonucleotide
tcgtaccttacatttagttaaa

SEQ ID NO: 33 - DNA, 22 bp, Artificial Sequence
  Synthetic deoxyribonucleotide
acgtaccgtacaaaacctgcct

SEQ ID NO: 34 - protein, 11 aa, Artificial Sequence
  Synthetic peptide
YGRKKRRQRRR

SEQ ID NO: 35 - protein, 9 aa, Artificial Sequence
  Synthetic peptide
RKKRRQRRR

SEQ ID NO: 36 - protein, 34 aa, Artificial Sequence
  Synthetic peptide
DAATATRGRSAASRPTERPRAPARSASRPRRPVE

SEQ ID NO: 37 - protein, 16 aa, Artificial Sequence
  Synthetic peptide
RRWRRWWRRWWRRWRR

SEQ ID NO: 38 - protein, 7 aa, Artificial Sequence
  Synthetic peptide
RRRRRRR

SEQ ID NO: 39 - protein, 10 aa, Artificial Sequence
  Synthetic peptide
RPKKRKVRRR

SEQ ID NO: 40 - protein, 28 aa, Artificial Sequence
  Synthetic peptide
GALFLGFLGGAAGSTMGAWSQPKSKRKV

SEQ ID NO: 41 - protein, 26 aa, Artificial Sequence
  Synthetic peptide
DPKGDPKGVTVTVTVTVTGKGDPKPD

SEQ ID NO: 42 - protein, 18 aa, Artificial Sequence
  Synthetic peptide
KLALKLALKALKAALKLA

SEQ ID NO: 43 - protein, 18 aa, Artificial Sequence
  Synthetic peptide
LLIILRRRIRKQAHAHSK

SEQ ID NO: 44 - protein, 16 aa, Artificial Sequence
  Synthetic peptide
RVIRVWFQNKRCKDKK

SEQ ID NO: 45 - protein, 21 aa, Artificial Sequence
  Synthetic peptide
KETWWETWWTEWSQPKKKRKV

SEQ ID NO: 46 - protein, 28 aa, Artificial Sequence
  Synthetic peptide
MANLGYWLLALFVTMWTDVGLCKKRPKP

SEQ ID NO: 47 - RNA, 25 nt, Artificial Sequence
  Synthetic ribonucleotide
uugucgaucucucuguuguccgagg

SEQ ID NO: 48 - RNA, 24 nt, Homo sapiens
guugcagagcauuaacgguacgcu

SEQ ID NO: 49 - RNA, 21 nt, Artificial Sequence
  Synthetic Ribonucleic Acid
cgcauuauugacacgguacga

SEQ ID NO: 50 - RNA, 21 nt, Artificial Sequence
  Synthetic Ribonucleic Acid
gcauuauugacacgguacgag

SEQ ID NO: 51 - RNA, 21 nt, Artificial Sequence
  Synthetic Ribonucleic Acid
gcgcauuaugaccacgguacg

SEQ ID NO: 52 - RNA, 23 nt, Artificial Sequence
  Synthetic Ribonucleic Acid
gcgcauuaugaccacgguacgag

SEQ ID NO: 53 - RNA, 66 nt, Artificial Sequence
  Synthetic Ribonucleic Acid
cgcauuauuacucacgguacgacgcauuauuacucacgguacgacgcauuauuacucacgguacga

SEQ ID NO: 54 - RNA, 84 nt, Artificial Sequence
  Synthetic Ribonucleic Acid
cgcauuauugacacgguacgacgcauuauugacacgguacgacgcauuauugacacgguacgacgcauuauugacacgguacga

SEQ ID NO: 55 - RNA, 66 nt, Artificial Sequence
  Synthetic Ribonucleic Acid
gcauuauuacucacgguacgaggcauuauuacucacgguacgaggcauuauuacucacgguacgag

SEQ ID NO: 56 - RNA, 105 nt, Artificial Sequence
  Synthetic Ribonucleic Acid
gcauuauugacacgguacgaggcauuauugacacgguacgaggcauuauugacacgguacgaggcauuauugacacgguacgaggcauuauugacacgguacgag

(19) United States
(12) Patent Application Publication
(10) Pub. No.: US 2011/0172293 A1
(43) Pub. Date: Jul. 14, 2011

(54) Methods and Compositions for Modulating Angiogenesis

(76) Inventors: Jason E. Fish, San Mateo, CA (US); Deepak Srivastava, San Francisco, CA (US)

(21) Appl. No.: 13/001,604

(22) PCT Filed: Jul. 1, 2009
(86) PCT No.: PCT/US09/49438

§ 371 Date: Mar. 21, 2011

Related U.S. Application Data
(60) Provisional application No. 61/134,352, filed on Jul. 8, 2008.

(51) Int. Cl. A61K 31/7088; C07H 21/04; C12N 15/63; A61P 35/00

(52) U.S. Cl. 514/44 A; 536/23.1; 514/44 R; 536/24.5; 435/320.1

(57) ABSTRACT

The present invention provides compositions comprising antisense nucleic acids that reduce miR-126 levels in an endothelial cell. The present invention provides compositions comprising a target protector nucleic acid. The present invention provides methods of modulating angiogenesis in an individual, the methods generally involving administering to the individual an effective amount of an agent that increases or that decreases the level of miR-126 in endothelial cells of the individual.

### CROSS-REFERENCE

This application claims the benefit of U.S. Provisional Patent Application No. 61/134,352, filed Jul. 8, 2008, which application is incorporated herein by reference in its entirety.

### BACKGROUND

The vascular network is composed of an intricate series of vessels that serve as conduits for blood flow, regulate organ growth, and modulate the response to injury. It is also requisite for expansion of tumor masses, and inhibition of vessel formation prevents tumor growth.

Vascular endothelial cells initially differentiate from angioblastic precursors and proliferate and migrate to form the primitive vascular plexus through the process of vasculogenesis. This network is further remodeled by angiogenesis and stabilized by recruitment of pericytes and vascular smooth muscle cells to form a functioning circulatory system. Several angiogenic stimuli are essential to establish the circulatory system during development and to control physiologic and pathologic angiogenesis in the adult. For example, secreted growth factors, including members of the vascular endothelial growth factor (VEGF), platelet-derived growth factor (PDGF), and fibroblast growth factor (FGF) families, bind to membrane-bound receptors and transmit signals through kinase-dependent signaling cascades. These signals ultimately result in gene expression changes that affect the growth, migration, morphology, and function of endothelial cells.

MicroRNAs are transcribed by RNA polymerase II as parts of longer primary transcripts known as pri-microRNAs. Pri-microRNAs are subsequently cleaved by Drosha, a double-stranded-RNA-specific ribonuclease, to form microRNA precursors or pre-microRNAs. Pre-microRNAs are exported from the nucleus into the cytoplasm where they are processed by Dicer. Dicer is a member of the RNase III family of nucleases that cleaves the pre-microRNA, resulting in a double-stranded RNA with overhangs, at both 3′ termini, that are one to four nucleotides long. The mature microRNA is derived from either the leading or the lagging arm of the microRNA precursor. The miRNA can bind a target mRNA and inhibit translation of the bound mRNA.

There is a need in the art for methods of modulating angiogenesis.

### Literature

Taniguchi et al. (2007) Mol. Cell Biol. 27:4541; Musiyenko et al. (2008) J. Mol. Med. 86:313; Harris et al. (2008) Proc. Natl. Acad. Sci. USA 105:1516; Musiyenko et al. (2008) J. Mol. Med. 86:313; WO 2007/112754; U.S. Patent Publication No. 2009/0131354.

### SUMMARY OF THE INVENTION

The present disclosure provides compositions comprising antisense nucleic acids that reduce miR-126 levels in an endothelial cell. The present disclosure provides compositions comprising a target protector nucleic acid. The present disclosure provides methods of modulating angiogenesis in an individual, the methods generally involving administering to the individual an effective amount of an agent that increases or that decreases the level of miR-126 in endothelial cells of the individual.

### BRIEF DESCRIPTION OF THE DRAWINGS

FIGS. 1A-C present data indicating that miR-126 is not sufficient for the differentiation of pluripotent cells to the endothelial cell lineage.

FIGS. 2A-E depict microRNAs enriched in endothelial cells.

FIGS. 3A-D depict effects of miR-126 on endothelial migration and capillary tube stability in vitro.

FIGS. 4A-E depict phenotypic analysis of endothelial cells with altered miR-126 expression.

FIGS. 5A-G depict effects of miR-126 on vascular integrity and lumen maintenance in vivo.

FIGS. 6A and 6B depict miR126 nucleotide sequence, the position of antisense morpholinos (MOs), and target sequences. FIG. 6A depicts a miR126 nucleotide sequence (SEQ ID NO:18) from Danio rerio, and the position of miR-126 MO-1 and miR-126 MO-2 antisense morpholinos used to block miR-126/126* expression in zebrafish. FIG. 6B depicts miR-126 (SEQ ID NO:2) binding sites in predicted human miR-126 target mRNAs SPRED1 (SEQ ID NO:24) CRK (SEQ ID NO:25), RGS3 (SEQ ID NO:26), ITGA6 (SEQ ID NO:27), PIK3R2 (SEQ ID NO:28), and VCAM1 (SEQ ID NO:29).

FIGS. 7A and 7B depict a feed-back loop involving miR-126 regulates EGFL7 expression.

FIGS. 8A-G depict miR-126 mRNA targets. FIG. 4E presents a Danio rerio miR-126 (dre-miR-126) nucleotide sequence (SEQ ID NO:2) and a spred1 mRNA target sequence (SEQ ID NO:48).

FIGS. 9A-D depict effects of miR-126 on SPRED1 and PIK3R2.

FIG. 10 depicts data showing that Spred1 is expressed in zebrafish endothelial cells.

FIGS. 11A-F depict effects of Spred1 on vascular instability and hemorrhage.

FIGS. 12A-D depict nucleotide sequences of miR-126 nucleic acids.

FIGS. 13A and 13B depict nucleotide sequences of exemplary target protector nucleic acids.

FIG. 14 depicts a nucleotide sequence of a SPRED1 mRNA.

FIGS. 15A and 15B depict a nucleotide sequence of a PIK3R2 mRNA.

FIGS. 16A and 16B depict the effect of a miR-126 antagomir on angiogenesis in vivo.

### DEFINITIONS

As used throughout, “modulation” is meant to refer to an increase or a decrease in the indicated phenomenon (e.g., modulation of a biological activity refers to an increase in a biological activity or a decrease in a biological activity). Accordingly, “modulation” of angiogenesis includes an increase or a decrease in angiogenesis.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art.

As used herein, the term “microRNA” refers to any type of interfering RNAs, including but not limited to, endogenous microRNAs and artificial microRNAs (e.g., synthetic miRNAs). Endogenous microRNAs are small RNAs naturally encoded in the genome which are capable of modulating the productive utilization of mRNA. An artificial microRNA can be any type of RNA sequence, other than endogenous microRNA, which is capable of modulating the activity of an mRNA. A microRNA sequence can be an RNA molecule composed of any one or more of these sequences. MicroRNA (or “miRNA”) sequences have been described in publications such as, Lim, et al., 2003, Genes & Development, 17, 991-1008, Lim et al., 2003, Science, 299, 1540, Lee and Ambrose, 2001, Science, 294, 862, Lau et al., 2001, Science 294, 858-861, Lagos-Quintana et al., 2002, Current Biology, 12, 735-739, Lagos-Quintana et al., 2001, Science, 294, 853-857, and Lagos-Quintana et al., 2003, RNA, 9, 175-179, which are incorporated herein by reference. Examples of microRNAs include any RNA that is a fragment of a larger RNA or is a miRNA, siRNA, stRNA, sncRNA, tncRNA, snoRNA, smRNA, snRNA, or other small non-coding RNA. See, e.g., US Patent Applications 20050272923, 20050266552, 20050142581, and 20050075492. A “microRNA precursor” (or “pre-miRNA”) refers to a nucleic acid having a stem-loop structure with a microRNA sequence incorporated therein. A “mature microRNA” (or “mature miRNA”) includes a microRNA that has been cleaved from a microRNA precursor (a “pre-miRNA”), or that has been synthesized (e.g., synthesized in a laboratory by cell-free synthesis), and has a length of from about 19 nucleotides to about 27 nucleotides, e.g., a mature microRNA can have a length of 19 nt, 20 nt, 21 nt, 22 nt, 23 nt, 24 nt, 25 nt, 26 nt, or 27 nt. A mature microRNA can bind to a target mRNA and inhibit translation of the target mRNA.

A “stem-loop structure” refers to a nucleic acid having a secondary structure that includes a region of nucleotides which are known or predicted to form a double strand (step portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. The actual primary sequence of nucleotides within the stem-loop structure is not critical to the practice of the invention as long as the secondary structure is present. As is known in the art, the secondary structure does not require exact base-pairing. Thus, the stem may include one or more base mismatches. Alternatively, the base-pairing may be exact, i.e. not include any mismatches.

A “small interfering” or “short interfering RNA” or siRNA is a RNA duplex of nucleotides that is targeted to a gene of interest (a “target gene”). An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length.

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Other examples are well known to those of skill in the art.

A nucleobase may be comprised in a nucleoside or nucleotide, using any chemical or natural synthesis method described herein or known to one of ordinary skill in the art. Such nucleobase may be labeled or it may be part of a molecule that is labeled and contains the nucleobase.

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar.

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

A nucleic acid is “hybridizable” to another nucleic acid, such as a cDNA, genomic DNA, or RNA, when a single stranded form of the nucleic acid can anneal to the other nucleic acid under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization. Hybridization conditions and post-hybridization washes are useful to obtain the desired determine stringency conditions of the hybridization. One set of illustrative post-hybridization washes is a series of washes starting with 6×SSC (where SSC is 0.15 M NaCl and 15 mM citrate buffer), 0.5% SDS at room temperature for 15 minutes, then repeated with 2×SSC, 0.5% SDS at 45° C. for 30 minutes, and then repeated twice with 0.2×SSC, 0.5% SDS at 50° C. for 30 minutes. Other stringent conditions are obtained by using higher temperatures in which the washes are identical to those above except for the temperature of the final two 30 minute washes in 0.2×SSC, 0.5% SDS, which is increased to 60° C. Another set of highly stringent conditions uses two final washes in 0.1×SSC, 0.1% SDS at 65° C. Another example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. Stringent hybridization conditions and post-hybridization wash conditions are hybridization conditions and post-hybridization wash conditions that are at least as stringent as the above representative conditions.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of the melting temperature (Tm) for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; and at least about 30 nucleotides. Furthermore, the skilled artisan will recognize that the temperature and wash solution salt concentration may be adjusted as necessary according to factors such as length of the probe.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm nih.gov/BLAST. See, e.g., Altschul et al. (1990), J. Mol. Biol. 215:403-10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

“Complementary,” as used herein, refers to the capacity for precise pairing between two nucleotides of a polynucleotide (e.g., an antisense polynucleotide) and its corresponding target polynucleotide. For example, if a nucleotide at a particular position of a polynucleotide is capable of hydrogen bonding with a nucleotide at a particular position of a target nucleic acid (e.g., a microRNA), then the position of hydrogen bonding between the polynucleotide and the target polynucleotide is considered to be a complementary position. The polynucleotide and the target polynucleotide are complementary to each other when a sufficient number of complementary positions in each molecule are occupied by nucleotides that can hydrogen bond with each other. Thus, “specifically hybridizable” and “complementary” are terms which are used to indicate a sufficient degree of precise pairing or complementarity over a sufficient number of nucleotides such that stable and specific binding occurs between the polynucleotide and a target polynucleotide.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A subject polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. As such, an antisense polynucleotide which is 18 nucleotides in length having 4 (four) noncomplementary nucleotides which are flanked by two regions of complete complementarity with the target nucleic acid would have 77.8% overall complementarity with the target nucleic acid. Percent complementarity of an oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, a human, a non-human primate, a rodent (e.g., a mouse, a rat, etc.), a lagomorph, an ungulate, a canine, a feline, etc. In some embodiments, a subject of interest is a human.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an antisense RNA” includes a plurality of such antisense RNAs and reference to “the miR-126 nucleic acid” includes reference to one or more miR-126 nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides compositions comprising antisense nucleic acids that reduce miR-126 levels in an endothelial cell. The present disclosure further provides target protector nucleic acids that bind to a miR-126 target mRNA. The present disclosure provides methods of modulating angiogenesis in an individual, the methods generally involving administering to the individual an effective amount of an agent that increases or that decreases the level of miR-126 in endothelial cells of the individual.

### Nucleic Acids

The present disclosure provides antisense nucleic acids, nucleic acids encoding the antisense nucleic acids, and composition comprising the antisense nucleic acids, where the nucleic acids modulate angiogenesis. The present disclosure further provides target protector nucleic acids that bind to a miR-126 target mRNA, and compositions comprising the target protector nucleic acids.

### Antisense Nucleic Acids

The present disclosure provides antisense nucleic acids, nucleic acids encoding the antisense nucleic acids, and composition comprising the antisense nucleic acids, where the nucleic acids modulate angiogenesis. A subject antisense nucleic acid is in some embodiments a DNA. A subject antisense nucleic acid is in some embodiments an RNA. A subject antisense nucleic acid is in some embodiments a peptide nucleic acid (PNA), a morpholino nucleic acid (MO), a locked nucleic acid (LNA), or some other form of nucleic acid, as described in more detail below. In some embodiments, a subject antisense nucleic acid comprises a nucleotide sequence capable of forming a stable duplex with a ribonuclease III cleavage site-containing portion of a miR-126 precursor nucleic acid. Ribonuclease III cleavage sites include Dicer cleavage sites and Drosha cleavage sites.

A subject antisense nucleic acid in some embodiments forms a stable duplex with a ribonuclease III cleavage site (e.g., a Drosha cleavage site, or a Dicer cleavage site) present in a miR-126 precursor nucleic acid. A subject antisense nucleic acid reduces the level of mature miR-126 nucleic acid in an endothelial cell by at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more than 90%, compared to the level of mature miR-126 nucleic acid in the endothelial cell in the absence of the antisense nucleic acid.

Drosha cleaves pri-microRNA at the base of a stem-loop structure, releasing the stem-loop structure. Helvik et al. (2007) Bioinformatics 23:142; Zeng et al. (2005) EMBO J. 24:138; MacRae and Doudna (2007) Curr. Opinion Structural Biol. 17:138.

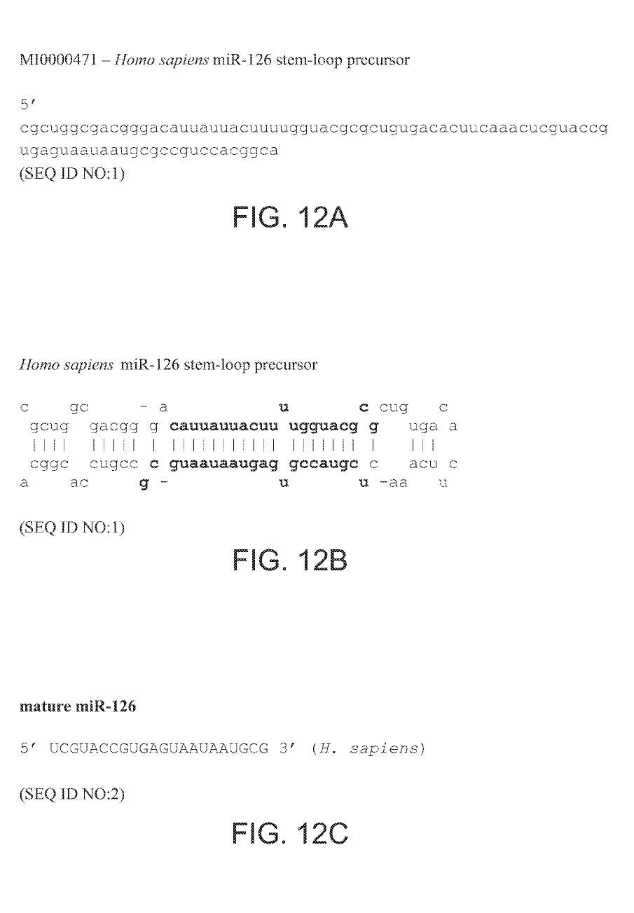

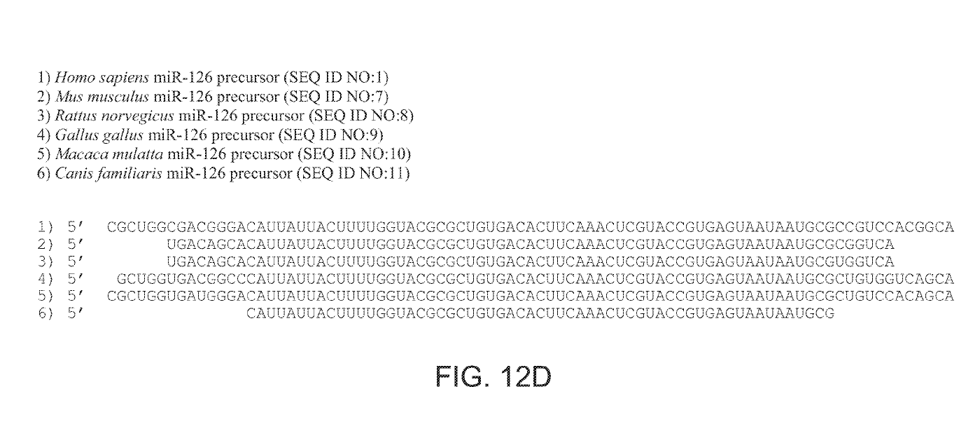

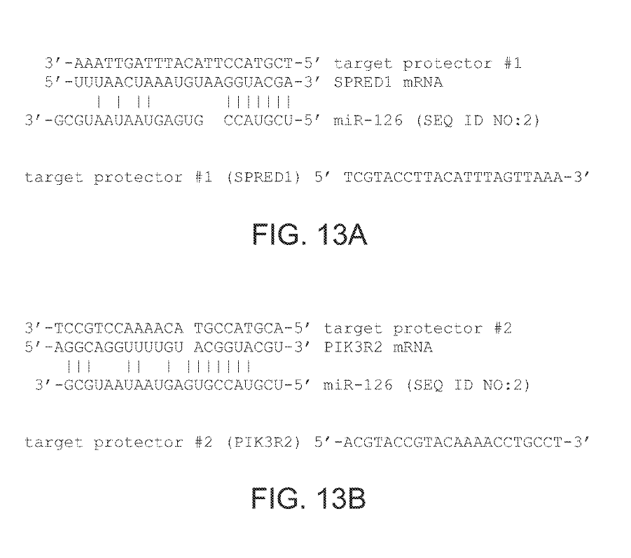

Dicer recognizes double-stranded RNA. MacRae and Doudna (2007) Curr. Opinion Structural Biol. 17:138. Thus, a Dicer cleavage site is located within the double-stranded portion of a miR-126 precursor nucleic acid, e.g., as depicted in FIG. 12B (SEQ ID NO:1). For example, a Dicer cleavage site is found in nucleotides 15 through 41, and in nucleotides 45 through 74, of the nucleotide sequence depicted in FIG. 12A (SEQ ID NO:1).

A miR-126 precursor nucleic acid comprises a nucleotide sequence having at least about 75%, at least about 80%, at least about 85%, at least about 90%, at least about 95%, at least about 98%, at least about 99%, or 100%, nucleotide sequence identity to the nucleotide sequence depicted in FIG. 12A (SEQ ID NO:1). The nucleotide sequence depicted in FIG. 12A is Homo sapiens miR-126 precursor nucleic acid. For example, a miR-126 precursor nucleic acid comprises a nucleotide sequence having at least about 75%, at least about 80%, at least about 85%, at least about 90%, at least about 95%, at least about 98%, at least about 99%, or 100%, nucleotide sequence identity to a contiguous stretch of 60 nucleotides of the nucleotide sequence from 15 to 74 of the nucleotide sequence depicted in FIG. 12A (SEQ ID NO:1). As shown in FIG. 12D, there is a high degree of nucleotide sequence identity among miR-126 precursor nucleic acids of various species to the nucleotide sequence from 15 to 74 of the nucleotide sequence depicted in FIG. 12A (H. sapiens miR-126 precursor; SEQ ID NO:1).

A suitable antisense nucleic acid comprises a nucleotide sequence that is complementary to nucleotides 15 through 41, nucleotides 45 through 74, nucleotides 14 through 40, nucleotides 14 through 41, nucleotides 16 through 42, nucleotides 44 through 74, nucleotides 45 through 71, nucleotides 45 through 72, nucleotides 45 through 73, nucleotides 52 through 73, or other similar portion, of the nucleotide sequence depicted in FIG. 12A (SEQ ID NO:1). A suitable antisense nucleic acid comprises a nucleotide sequence having fewer than five mismatches in complementarity with nucleotides 15 through 41, nucleotides 45 through 74, nucleotides 14 through 40, nucleotides 14 through 41, nucleotides 16 through 42, nucleotides 44 through 74, nucleotides 45 through 71, nucleotides 45 through 72, nucleotides 45 through 73, nucleotides 52 through 73, or other similar portion, of the nucleotide sequence depicted in FIG. 12A (SEQ ID NO:1). Thus, e.g., a suitable antisense nucleic acid can comprise a nucleotide sequence that has 1, 2, 3, or 4 mismatches in complementarity with nucleotides 15 through 41, nucleotides 45 through 74, nucleotides 14 through 40, nucleotides 14 through 41, nucleotides 16 through 42, nucleotides 44 through 74, nucleotides 45 through 71, nucleotides 45 through 72, nucleotides 45 through 73, nucleotides 52 through 73, or other similar portion, of the nucleotide sequence depicted in FIG. 12A (SEQ ID NO:1).

The portion of a subject antisense nucleic acid that forms a duplex with a miR-126 precursor nucleic acid (e.g., the portion of a subject antisense nucleic acid that forms a duplex with nucleotides 15 through 41, nucleotides 45 through 74, nucleotides 14 through 40, nucleotides 14 through 41, nucleotides 16 through 42, nucleotides 44 through 74, nucleotides 45 through 71, nucleotides 45 through 72, nucleotides 45 through 73, nucleotides 52 through 73, or other similar portion, of the nucleotide sequence depicted in FIG. 12A (SEQ ID NO:1)) has a length of from about 20 nucleotides to about 50 nucleotides. For example, a subject antisense nucleic acid can have a length of from about 20 nt to about 50 nt. One having ordinary skill in the art will appreciate that this embodies antisense nucleic acids having a length of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

The total length of a subject antisense nucleic acid can be greater than the duplex-forming portion, e.g., the total length of a subject antisense nucleic acid can be from about 20 nucleotides (nt) to about 30 nt, from about 30 nt to about 40 nt, from about 40 nt to about 50 nt, from about 50 nt to about 75 nt, from about 75 nt to about 100 nt, from about 100 nt to about 125 nt, from about 125 nt to about 150 nt, from about 150 nt to about 175 nt, or from about 175 nt to about 200 nt, or greater than 200 nt, in length.

Exemplary, non-limiting nucleotide sequences that can be included in a subject antisense nucleic acid are as follows:

(SEQ ID NO: 12)
1) 5′-tcacagcgcgtaccaaaagtaataatg-3′;
(SEQ ID NO: 13)
2) 5′-gcgcattattactcacggtacgagtttgaa-3′;
(SEQ ID NO: 14)
3) 5′-gtcacagcgcgtaccaaaagtaataatg-3′;
(SEQ ID NO: 15)
4) 5′-tcacagcgcgtaccaaaagtaataatgtcc-3′;
(SEQ ID NO: 16)
5) 5′-cattattactcacggtacgagtttgaa-3′;
and
(SEQ ID NO: 17)
6) 5′-cgcattattactcacggtacgagtttgaa-3′.

In some embodiments, a subject antisense nucleic acid is referred to as an antagomir. Krützfeldt et al. (2005) Nature 438:685. A subject antisense nucleic acid can include one or more 2′-O-methyl (2′-OMe) sugar modifications. A subject antisense can include one or more phosphate backbone modifications, e.g., phosphorothioate, phosphoroamidate, etc. A subject antisense nucleic acid can include a cholesterol moiety conjugated to the nucleic acid, e.g., at the 3′ end of the nucleic acid. Cholesterol can be linked to a 2′-O-methyl-oligoribonucleotide (2′-OMe-RNA) via a disulfide bond by reacting the 3′-(pyridyldithio)-modified 2′-OMe-RNA with thiocholesterol in dichloromethane-methanol solution. See, e.g., Oberhauser and Wagner (1992) Nucl. Acids Res. 20:533. Cholesterol can be linked to the 3′ end of a nucleic acid via a hydroxyprolinol linkage. See, e.g., Krützfeldt et al. (2005) Nature 438:685.

Exemplary, non-limiting nucleotide sequences that can be included in a subject antisense nucleic acid include:

| | |
|---|---|
| 5′-cgcauuauuacucacgguacga-3′; | (SEQ ID NO: 3) |
| 5′-gcauuauuacucacgguacgag-3′; | (SEQ ID NO: 4) |
| 5′-gcgcauuauuacucacgguacg-3′; | (SEQ ID NO: 5) |

and

| | |
|---|---|
| 5′-gcgcauuauuacucacgguacgag-3′. | (SEQ ID NO: 6) |

In some embodiments, a subject antisense nucleic acid has a length of from about 20 nt to about 25 nt, where one or more (in some cases all) of the nucleotides includes a 2′-OMe modification, where one or more (in some cases all) of the phosphate backbone linkages includes phosphorothioate linkages, and where the 3′ end of the nucleic acid comprises a cholesterol moiety covalently linked (e.g., via a hydroxyprolinol linkage). A subject antisense nucleic acid can also be a PNA, a LNA, or some other form of nucleic acid.

### Competitive Inhibitor Nucleic Acids

The present disclosure provides nucleic acids (e.g., synthetic nucleic acids) that are competitive inhibitors of a miR-126 nucleic acid (e.g., a naturally-occurring endogenous miR-126 nucleic acid) and that reduce the activity of a miR-126 nucleic acid. These competitive inhibitor nucleic acids are also referred to as “microRNA sponges.” A subject competitive inhibitor nucleic acid comprises multiple, tandem binding sites to a miR-126 nucleic acid. The present disclosure also provides a vector nucleic acid comprising a nucleotide sequence encoding a subject competitive inhibitor of a miR-126 nucleic acid.

A subject competitive inhibitor nucleic acid can inhibit binding of a miR-126 nucleic acid with a target nucleic acid in an endothelial cell. For example, a subject competitive inhibitor nucleic acid can inhibit binding of a miR-126 nucleic acid with a target nucleic acid in an endothelial cell by at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more than 90%, compared to the binding of the miR-126 to the target nucleic acid in the cell in the absence of the competitive inhibitor nucleic acid.

In some embodiments, a subject competitive inhibitor nucleic acid has the structure 5′-Xₘ-(A)ₙ-X′ₚ-3′, where X and X′ are optional flanking nucleotides; A is a nucleotide sequence that is complementary to a miR-126 nucleic acid (e.g., to a mature miR-126 nucleic acid); m and p are independently an integer from 1 to about 50 or greater than 50 (e.g., from about 50 to about 100, from about 100 to about 150, from about 150 to about 200, from about 200 to about 500, or greater than 500); and n is an integer from 2 to about 20 (e.g., 2, 3, 4, 5, 6, 7, 8, 9, 10-15, or 15-20), or greater than 20 (e.g., from about 20 to about 25, from about 25 to about 30, from about 30 to about 40, from about 40 to about 50, or greater than 50). In some embodiments, the nucleotide sequence that is complementary to a miR-126 nucleic acid has a length of from about 15 nucleotides (nt) to about 25 nt (e.g., 15, 16, 17, 18, 19, 20, 21, 22, 23, 24, or 25 nt), and has at least about 75%, at least about 80%, at least about 85%, at least about 90%, at least about 95%, at least about 98%, at least about 99%, or 100%, nucleotide sequence identity to the complement of SEQ ID NO:2 (5′ UCGUACCGUGAGUAAUAAUGCG 3′).

In some embodiments, a subject competitive inhibitor nucleic acid includes a “bulge” in or around the center of the nucleotide sequence that is complementary to a miR-126 nucleic acid, where the “bulge” is a region of 2 nt, 3 nt, 4 nt, 5 nt, or 6 nt of non-complementarity with the miR-126 nucleic acid. Such a region of non-complementarity in or around the center of the nucleotide sequence that is complementary to a miR-126 nucleic acid reduces RNA interference-type cleavage and degradation of the competitive inhibitor nucleic acid.

Exemplary nucleotide sequences that are complementary to a miR-126 nucleic acid, and that can be included in a subject competitive inhibitor nucleic acid, include, but are not limited to:

| | |
|---|---|
| 5′-cgcauuauuacucacgguacga-3′; | (SEQ ID NO: 3) |
| 5′-gcauuauuacucacgguacgag-3′; | (SEQ ID NO: 4) |
| 5′-gcgcauuauuacucacgguacg-3′; | (SEQ ID NO: 5) |
| 5′-gcgcauuauuacucacgguacgag-3′; | (SEQ ID NO: 6) |
| 5′-cgcauuauugacacgguacga-3′; | (SEQ ID NO: 49) |
| 5′-gcauuauugacacgguacgag-3′; | (SEQ ID NO: 50) |
| 5′-gcgcauuaugaccacgguacg-3′; | (SEQ ID NO: 51) |
| and | |
| 5′-gcgcauuaugaccacgguacgag-3′. | (SEQ ID NO: 52) |

In some embodiments, a subject competitive inhibitor nucleic acid has the structure 5′-Xₘ-(A)ₙ-X′ₚ-3′, where X, X′, m, and p are as described above, and (A)ₙ has one of following exemplary, non-limiting sequences:

(SEQ ID NO: 53)
5′-cgcauuauuacucacgguacga cgcauuauuacucacgguacga
cgcauuauuacucacgguacga-3′,
i.e., three tandem repeats of SEQ ID NO: 3;
(SEQ ID NO: 54)
5′-cgcauuauugacacgguacga cgcauuauugacacgguacga
cgcauuauugacacgguacga cgcauuauugacacgguacga-3′,
i.e., four tandem repeats of SEQ ID NO: 49);
(SEQ ID NO: 55)
5′-gcauuauuacucacgguacgag gcauuauuacucacgguacgag
gcauuauuacucacgguacgag-3′,
i.e., three tandem repeats of SEQ ID NO: 4; and
(SEQ ID NO: 56)
5′-gcauuauugacacgguacgag gcauuauugacacgguacgag
gcauuauugacacgguacgag gcauuauugacacgguacgag
gcauuauugacacgguacgag-3′,
i.e., five tandem repeats of SEQ ID NO: 50).

As noted above, the present disclosure provides a recombinant vector comprising a nucleotide sequence encoding a subject competitive inhibitor nucleic acid, where the nucleotide sequence encoding a subject competitive inhibitor nucleic acid is operably linked to a promoter that is functional in a eukaryotic cell (e.g., a mammalian cell, e.g., a mammalian endothelial cell). A subject recombinant vector, when present in a mammalian cell (e.g., a mammalian endothelial cell) provides for production of a subject competitive inhibitor nucleic acid in the cell. In some embodiments, the promoter is an endothelial cell-specific promoter (described elsewhere herein). In some embodiments, the promoter is a strong RNA Polymerase III promoter. In some embodiments, the promoter is an RNA Polymerase III U6 promoter. Suitable vectors are known to those skilled in the art. Exemplary vectors are described elsewhere herein. See also Ebert et al. (2007) Nature Methods 4:721 for non-limiting examples of promoters and vectors suitable for use in expressing an miRNA “sponge” competitive inhibitor nucleic acid in a cell.

### Target Protector Nucleic Acids

The present disclosure provides a synthetic target protector nucleic acid that binds to a miR-126 target mRNA. A subject target protector nucleic acid does not induce cleavage or translational repression of the target mRNA; however, a subject target protector nucleic acid does inhibit binding of a miR-126 to the miR-126 target mRNA.

A subject synthetic target protector nucleic acid reduces miR-126-mediated inhibition of translation of a target mRNA by at least about 10%, at least about 20%, at least about 25%, at least about 30%, at least about 40%, at least about 50%, at least about 60%, at least about 70%, at least about 80%, or at least about 90%, or more than 90%, compared to the level of miR-126-mediated inhibition of the target mRNA in the absence of the synthetic target protector nucleic acid.

Where the miR-126 target mRNA is a negative regulator of angiogenic signaling (a negative regulator of angiogenesis), a subject synthetic target protector nucleic acid reduces miR-126-mediated inhibition of translation of the negative regulator, thereby increasing the levels in a cell of the negative regulator; in these cases, a subject synthetic target protector nucleic acid inhibits angiogenesis. Thus, for example, a subject synthetic target protector nucleic acid can result in at least about 10%, at least about 20%, at least about 25%, at least about 30%, at least about 40%, at least about 50%, at least about 60%, at least about 70%, at least about 80%, or at least about 90%, or more than 90%, inhibition of angiogenesis, e.g., where the synthetic target protector nucleic acid is introduced into an endothelial cell.

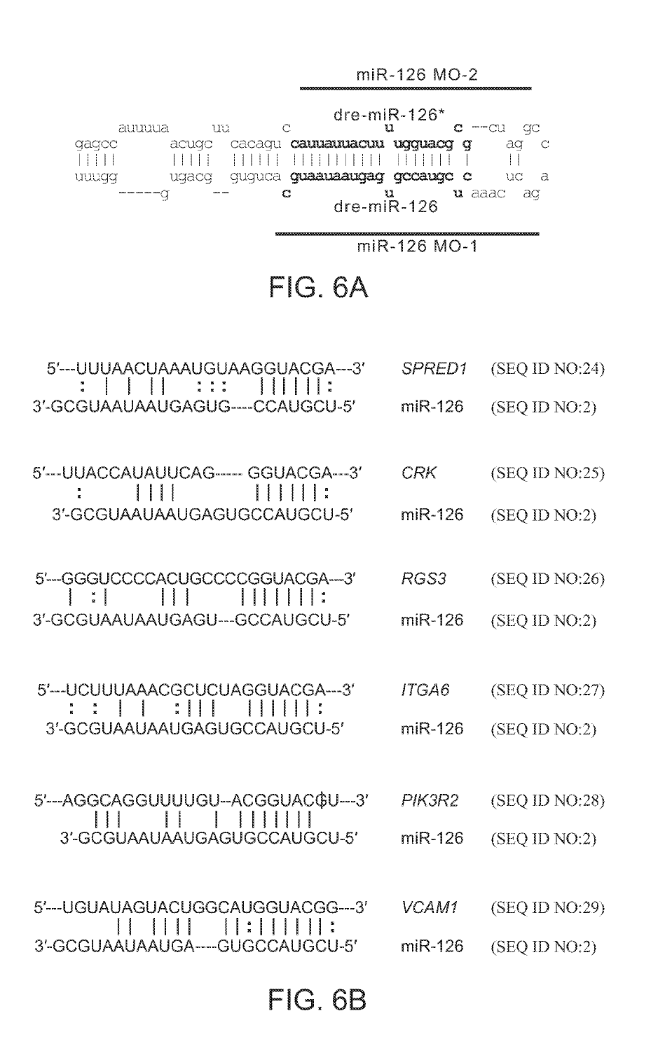

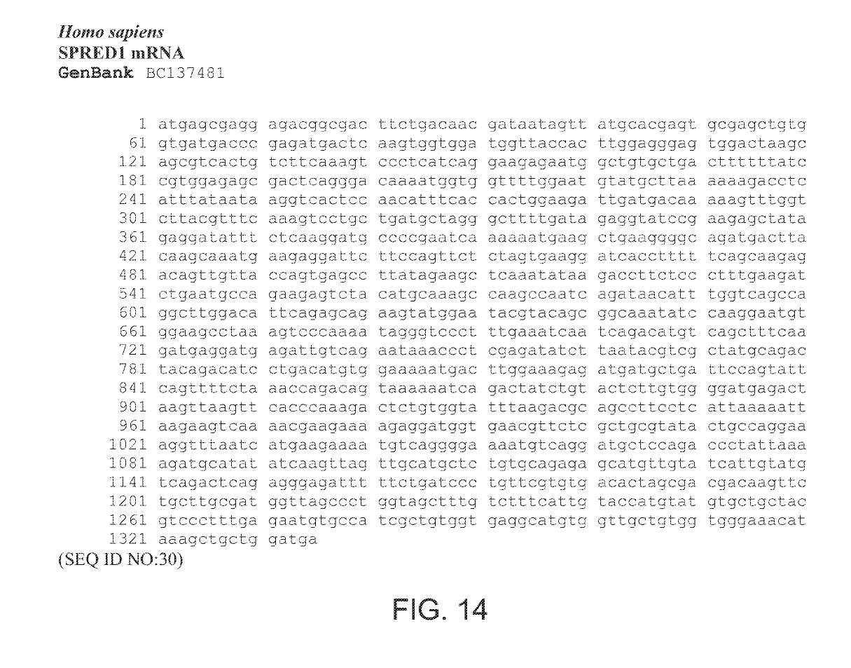

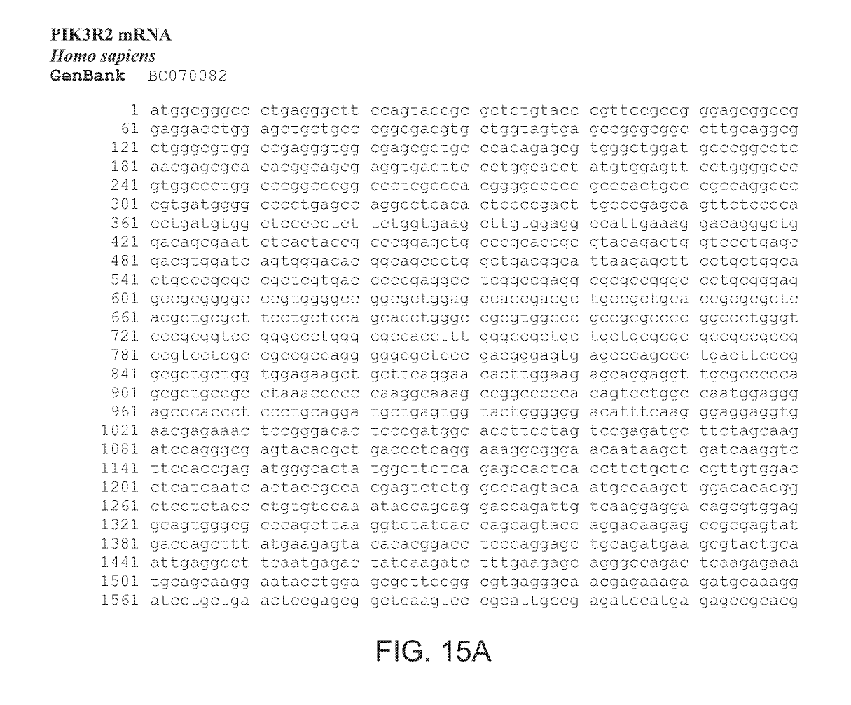

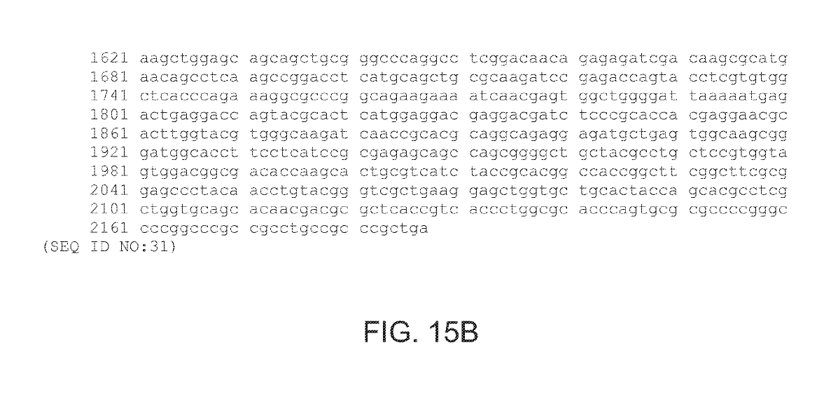

Target mRNAs that are targets for miR-126-mediated inhibition of translation include, e.g., SPRED1, PIK3R2, and VCAM1. Target sequences of these mRNA are depicted in FIG. 6B. Nucleotide sequences of miR-126 target mRNAs are known in the art. A SPRED1 mRNA can comprise a nucleotide sequence having a least about 85%, at least about 90%, at least about 95%, at least about 98%, at least about 99%, or 100% nucleotide sequence identity, to the nucleotide sequence (or the complement thereof) depicted in FIG. 14 (SEQ ID NO:30). A PIK3R2 mRNA can comprise a nucleotide sequence having a least about 85%, at least about 90%, at least about 95%, at least about 98%, at least about 99%, or 100% nucleotide sequence identity, to the nucleotide sequence (or the complement thereof) depicted in FIGS. 15A and 15B (SEQ ID NO:31).

A subject synthetic target protector nucleic acid can have a length of from about 19 nt to about 50 nt or more, e.g., a subject synthetic target protector nucleic acid can have a length of 19 nt, 20 nt, 21 nt, 22 nt, 23 nt, 24 nt, 25 nt, from 25 nt to about 30 nt, from about 30 nt to about 35 nt, from about 35 nt to about 40 nt, or from about 40 nt to about 50 nt, or longer than 50 nt.

As one non-limiting example, the target mRNA is a SPRED1 mRNA, and a subject synthetic target protector nucleic acid comprises a nucleotide sequence having at least about 85%, at least about 90%, at least about 95%, at least about 98%, at least about 99%, or 100% nucleotide sequence identity to the following nucleotide sequence: 5′ TCGTACCTTACATTTAGTTAAA-3′ (SEQ ID NO:32). For example, a subject synthetic target protector nucleic acid can have a length of 22 nt to about 25 nucleotides, and can comprise a nucleotide sequence having at least about 85%, at least about 90%, at least about 95%, at least about 98%, at least about 99%, or 100% nucleotide sequence identity to the following nucleotide sequence: 5′ TCGTACCTTACATTTAGTTAAA-3′ (SEQ ID NO:32).

As another non-limiting example, the target mRNA is a PIK3R2 mRNA, and a subject synthetic target protector nucleic acid comprises a nucleotide sequence having at least about 85%, at least about 90%, at least about 95%, at least about 98%, at least about 99%, or 100% nucleotide sequence identity to the following nucleotide sequence: 5′-ACGTACCGTACAAAACCTGCCT-3′ (SEQ ID NO:33). For example, a subject synthetic target protector nucleic acid can have a length of 22 nt to about 25 nucleotides, and can comprise a nucleotide sequence having at least about 85%, at least about 90%, at least about 95%, at least about 98%, at least about 99%, or 100% nucleotide sequence identity to the following nucleotide sequence: 5′-ACGTACCGTACAAAACCTGCCT-3′ (SEQ ID NO:33).

A subject synthetic target protector nucleic acid can be present in a composition, e.g., a pharmaceutical composition, as described in more detail below. In addition, as described in more detail below, a subject synthetic target protector nucleic acid can include one or more modifications (e.g., base modifications, linkage modifications, etc.).

### Recombinant Vectors

The present disclosure provides a nucleic acid comprising a nucleotide sequence encoding a subject antisense nucleic acid, a subject target protector nucleic acid, or a subject competitive inhibitor nucleic acid. In some embodiments, a nucleic acid comprising a nucleotide sequence encoding a subject antisense nucleic acid, a subject target protector nucleic acid, or a subject competitive inhibitor nucleic acid is a recombinant expression vector that provides for production of the encoded antisense nucleic acid, target protector nucleic acid, or competitive inhibitor nucleic acid in a cell (e.g., a eukaryotic cell, a mammalian cell, a mammalian endothelial cell).

A nucleotide sequence encoding a subject antisense nucleic acid, a subject target protector nucleic acid, or a subject competitive inhibitor nucleic acid can be included in an expression vector, resulting in a recombinant expression vector comprising a nucleotide sequence encoding a subject antisense nucleic acid, a subject target protector nucleic acid, or a subject competitive inhibitor nucleic acid. Expression vectors generally have convenient restriction sites located near the promoter sequence to provide for the insertion of a nucleic acid of interest. A selectable marker operative in the expression host may be present.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641648, 1999; Ali et al., Hum Mol Genet. 5:591594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; a lentivirus; a human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Suitable eukaryotic vectors include, for example, bovine papilloma virus-based vectors, Epstein-Barr virus-based vectors, vaccinia virus-based vectors, SV40, 2-micron circle, pcDNA3.1, pcDNA3.1/GS, pYES2/GS, pMT, p IND, pIND(Sp1), pVgRXR (Invitrogen), and the like, or their derivatives. Such vectors are well known in the art (Botstein et al., Miami Wntr. SyTnp. 19:265-274, 1982; Broach, In: “The Molecular Biology of the Yeast Saccharomyces: Life Cycle and Inheritance”, Cold Spring Harbor Laboratory, Cold Spring Harbor, N.Y., p. 445-470, 1981; Broach, Cell 28:203-204, 1982; Dilon et al., J. Clin. Hematol. Onco1.10:39-48, 1980; Maniatis, In: Cell Biology: A Comprehensive Treatise, Vol. 3, Gene Sequence Expression, Academic Press, NY, pp. 563-608, 1980.

The recombinant vector can include one or more coding regions that encode a polypeptide (a “selectable marker”) that allow for selection of the recombinant vector in a genetically modified host cell comprising the recombinant vector. Suitable selectable markers include those providing antibiotic resistance; e.g., blasticidin resistance, neomycin resistance. Several selectable marker genes that are useful include the hygromycin B resistance gene (encoding aminoglycoside phosphotranferase (APH)) that allows selection in mammalian cells by conferring resistance to hygromycin; the neomycin phosphotranferase gene (encoding neomycin phosphotransferase) that allows selection in mammalian cells by conferring resistance to G418; and the like.

In some embodiments, the recombinant vector integrates into the genome of the host cell (e.g., an endothelial cell); in other embodiments, the recombinant vector is maintained extrachromosomally in the host cell comprising the recombinant vector. A host cell (e.g., an endothelial cell) comprising a subject recombinant vector is a “genetically modified” host cell.

A nucleotide sequence encoding a subject antisense nucleic acid, a subject target protector nucleic acid, or a subject competitive inhibitor nucleic acid is operably linked to one or more transcriptional control elements, e.g., a promoter. Non-limiting examples of suitable eukaryotic promoters (promoters functional in a eukaryotic cell) include cytomegalovirus (CMV) immediate early, herpes simplex virus (HSV) thymidine kinase, early and late SV40, long terminal repeats (LTRs) from retrovirus, and mouse metallothionein-I. In some embodiments, the promoter is a constitutive promoter. Non-limiting examples of constitutive promoters include: ubiquitin promoter, CMV promoter, JeT promoter (U.S. Pat. No. 6,555,674), SV40 promoter, Elongation Factor 1 alpha promoter (EF1-alpha), RSV, and Mo-MLV-LTR. In some embodiments, the promoter is an inducible promoter. Non-limiting examples of inducible/repressible promoters include: Tet-On, Tet-Off, Rapamycin-inducible promoter, and Mx1. Selection of the appropriate vector and promoter is well within the level of ordinary skill in the art.

In some embodiments, the promoter is an endothelial cell-specific promoter. Endothelial cell-specific promoters include, e.g., a preproendothelin-1 (PPE-1) promoter, a PPE-1-3× promoter, a TIE-1 promoter, a TIE-2 promoter, an endoglin promoter, a von Willebrand factor (vWF) promoter, a KDR/flk-1 promoter, an endothelin-1 promoter, a FLT-1 promoter, an Egr-1 promoter, an ICAM-1 promoter, a VCAM-1 promoter, a PECAM-1 promoter, and an aortic carboxypeptidase-like protein (ACLP) promoter. Endothelial cell-specific promoters are known in the art; see, e.g., U.S. Pat. No. 5,888,765 (KDR/flk-1 promoter); U.S. Pat. No. 6,200,751 (endothelin-1 promoter); Cowan et al. (1998) J. Biol. Chem. 273:11737 (ICAM-2 promoter); Fadel et al. (1998) Biochem. J. 330:335 (TIE-2 promoter); and Dai et al. (2004) J. Virol. 78:6209 (synthetic EC-specific promoters); U.S. Pat. No. 7,067,649 (PPE-1 promoter); Varda-Bloom et al. (2001) Gene Ther. 8:819 (PPE-1 promoter); Velasco et al. (2001) Gene Ther. 8:897 (endoglin promoter; U.S. Pat. No. 6,103,527 (endoglin promoter); Ozaki et al. (1996) Hum. Gene Ther. 20:1483 (vWF promoter); and WO 2006/051545. Also suitable for use are a vascular-endothelial-cadherin (VE-Cadherin) promoter (Prandini et al, Oncogene, 2005, Apr. 21; 24(18):2992-3001); a MEF2C promoter (de Val et al, Cell, 2008, Dec. 12; 135(6); and an endothelial nitric oxide synthase (eNOS) promoter (Guillot et al, J. Clin Invest, 1999, March; 103(6):799-805). Also suitable are inducible versions of an endothelial cell-specific promoter; e.g., TIE-2 (Forde et al, Genesis, 2002, August; 33(4):191-7 and Deutsch et al, Exp Cell Res, 2008, Apr. 1; 314(6):1202-16) and VE-Cadherin (used in Hellstrom et al, Nature, 2007, Feb. 15; 445(7129):776-80)).

### Modifications

In some embodiments, a subject nucleic acid comprises one or more modifications, including phosphate backbone modifications, base modifications, sugar modifications, and other types of modifications. As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage

### Modified Backbones and Modified Internucleoside Linkages

Examples of suitable nucleic acids (e.g., a subject antisense nucleic acid; a subject synthetic target protector nucleic acid; a subject competitive inhibitor nucleic acid) containing modifications include nucleic acids containing modified backbones and/or non-natural internucleoside linkages. Nucleic acids (e.g., a subject antisense nucleic acid; a subject synthetic target protector nucleic acid) having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some embodiments, a subject nucleic acid (e.g., a subject antisense nucleic acid; a subject synthetic target protector nucleic acid; a subject competitive inhibitor nucleic acid) comprises one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in t U.S. Pat. No. 5,602,240.

In some embodiments, a subject nucleic acid (e.g., a subject antisense nucleic acid; a subject synthetic target protector nucleic acid) comprises one or more morpholino backbone structures as described in, e.g., U.S. Pat. No. 5,034,506. For example, in some embodiments, a subject nucleic acid (e.g., a subject antisense nucleic acid; a subject synthetic target protector nucleic acid) comprises a 6-membered morpholino ring in place of a ribose ring. In some of these embodiments, a phosphorodiamidate or other non-phosphodiester internucleoside linkage replaces a phosphodiester linkage Morpholino nucleic acids (“morpholinos”) include bases bound to morpholine rings instead of deoxyribose rings; in addition, the phosphate backbone can include a non-phosphate group, e.g., a phosphorodiamidate group instead of phosphates. Summerton (1999) Biochim. Biophys. Acta 1489:141; Heasman (2002) Dev. Biol. 243:209; Summerton and Weller (1997) Antisense & Nucl. Acid Drug Dev. 7:187; Hudziak et al. (1996) Antisense & Nucl. Acid Drug Dev. 6:267; Partridge et al. (1996) Antisense & Nucl. Acid Drug Dev. 6:169; Amantana et al. (2007) Bioconj. Chem. 18:1325; Morcos et al. (2008) BioTechniques 45:616.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Modifications that Facilitate Entry into a Mammalian Cell

In some embodiments, a subject nucleic acid comprises a moiety that facilitates entry into a mammalian cell. For example, in some embodiments, a subject nucleic acid comprises a cholesterol moiety covalently linked to the 3′ end of the nucleic acid. As another example, in some embodiments, a subject nucleic acid comprises a covalently linked peptide that facilitates entry into a mammalian cell. For example, a suitable peptide is an arginine-rich peptide. Amantana et al. (2007) Bioconj. Chem. 18:1325. As another example, in some embodiments, a subject nucleic acid comprises an octa-guanidinium dendrimer attached to the end of the nucleic acid. Morcos et al. (2008) BioTechniques 45:616.

### Mimetics

A subject nucleic acid (e.g., a subject antisense nucleic acid; a subject synthetic target protector nucleic acid, a subject competitive inhibitor nucleic acid) can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in an DNA/RNA molecule is replaced with a cyclohenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun, 1998, 4, 455-456). LNA and LNA analogs display very high duplex thermal stabilities with complementary DNA and RNA (Tm=+3 to +10 C), stability towards 3′-exonucleolytic degradation and good solubility properties. Potent and nontoxic antisense oligonucleotides containing LNAs have been described (Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

### Modified Sugar Moieties

A subject nucleic acid (e.g., a subject antisense nucleic acid; a subject synthetic target protector nucleic acid; a subject competitive inhibitor nucleic acid) can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂ CH₂OCH₃, also known as 2′-O-(2-methoxyethyl) or 2′-MOE) (Martin et al., Helv. Chim Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH₂CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

A subject nucleic acid (e.g., a subject antisense nucleic acid; a subject synthetic target protector nucleic acid; a subject competitive inhibitor nucleic acid) may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′: 4,5)pyrrolo[2,3-d]pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound (e.g., an antisense nucleic acid; a target protector nucleic acid). These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

### Conjugates

Another possible modification of a subject nucleic acid (e.g., a subject antisense nucleic acid; a subject synthetic target protector nucleic acid, a subject competitive inhibitor nucleic acid) involves chemically linking to the polynucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject antisense nucleic acid or target protector nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

In some embodiments, a subject nucleic acid is linked, covalently or non-covalently, to a cell penetrating peptide. Suitable cell penetrating peptides include those discussed in U.S. Patent Publication No. 2007/0129305. The cell penetrating peptides can be based on known peptides, including, but not limited to, penetratins; transportans; membrane signal peptides; viral proteins (e.g., Tat protein, VP22 protein, etc.); and translocating cationic peptides. Tat peptides comprising the sequence YGRKKRRQRRR (SEQ ID NO:34) are sufficient for protein translocating activity. Additionally, branched structures containing multiples copies of Tat sequence RKKRRQRRR (SEQ ID NO:35; Tung, C. H. et al., Bioorg. Med. Chem 10:3609-3614 (2002)) can translocate efficiently across a cell membrane. Variants of Tat peptides capable of acting as a cell penetrating agent are described in Schwarze, S. R. et al., Science 285:1569-1572 (1999). A composition containing the C-terminal amino acids 159-301 of HSV VP22 protein is capable of translocating different types of cargoes into cells. Translocating activity is observed with a minimal sequence of DAATATRGRSAASRPTERPRAPARSASRPRRPVE (SEQ ID NO:36). Active peptides with arginine rich sequences are present in the Grb2 binding protein, having the sequence RRWRRWWRRWWRRWRR (SEQ ID NO:37; Williams, E. J. et al., J. Biol. Chem. 272:22349-22354 (1997)) and polyarginine heptapeptide (SEQ ID NO:38; Chen, L. et al., Chem. Biol. 8:1123-1129 (2001); Futaki, S. et al., J. Biol. Chem. 276:5836-5840 (2001); and Rothbard, J. B. et al., Nat. Med. 6(11):1253-7 (2000)). An exemplary cell penetrating peptide has the sequence RPKKRKVRRR (SEQ ID NO:39), which is found to penetrate the membranes of a variety of cell types. Also useful are branched cationic peptides capable of translocation across membranes, e.g., (KKKK)₂GGC, (KWKK)₂GCC, and (RWRR)₂GGC (Plank, C. et al., Human Gene Ther. 10:319-332 (1999)). A cell penetrating peptide can comprise chimeric sequences of cell penetrating peptides that are capable of translocating across cell membrane. An exemplary molecule of this type is transportan GALFLGFLGGAAGSTMGAWSQPKSKRKV (SEQ ID NO:40), a chimeric peptide derived from the first twelve amino acids of galanin and a 14 amino acid sequence from mastoporan (Pooga, M et al., Nature Biotechnol. 16:857-861 (1998). Other types of cell penetrating peptides are the VT5 sequences DPKGDPKGVTVTVTVTVTGKGDPKPD (SEQ ID NO:41), which is an amphipathic, beta-sheet forming peptide (Oehlke, J., FEBS Lett. 415(2):196-9 (1997); unstructured peptides described in Oehlke J., Biochim Biophys Acta. 1330(1):50-60 (1997); alpha helical amphipathic peptide with the sequence KLALKLALKALKAALKLA (SEQ ID NO:42; Oehlke, J. et al., Biochim Biophys Acta. 1414(1-2):127-39 (1998); sequences based on murine cell adhesion molecule vascular endothelial cadherin, amino acids 615-632 LLIILRRRIRKQAHAHSK (SEQ ID NO:43; Elmquist, A. et al., Exp Cell Res. 269(2):237-44 (2001); sequences based on third helix of the islet 1 gene enhancer protein RVIRVWFQNKRCKDKK (SEQ ID NO:44; Kilk, K. et al., Bioconjug. Chem. 12(6):911-6 (2001)); amphipathic peptide carrier Pep-1 KETWWETWWTEWSQPKKKRKV (SEQ ID NO:45; Morris, M. C. et al., Nat. Biotechnol. 19(12):1173-6 (2001)); and the amino terminal sequence of mouse prion protein MANLGYWLLALFVTMWTDVGLCKKRPKP (SEQ ID NO:46; Lundberg, P. et al., Biochem. Biophys. Res. Commun 299(1):85-90 (2002).

### Compositions and Formulations

The present disclosure provides compositions, e.g., pharmaceutical compositions, comprising a subject nucleic acid (e.g., a subject antisense nucleic acid; a subject synthetic target protector nucleic acid; a subject competitive inhibitor nucleic acid; or a nucleic acid (e.g., a recombinant vector) comprising a nucleotide sequence encoding a subject antisense nucleic acid, a subject synthetic target protector nucleic acid, or a subject competitive inhibitor nucleic acid). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A subject composition can include: a) a subject nucleic acid (where a subject nucleic acid can be a subject antisense nucleic acid; a subject synthetic target protector nucleic acid; a subject competitive inhibitor nucleic acid; or a nucleic acid (e.g., a recombinant vector) comprising a nucleotide sequence encoding a subject antisense nucleic acid, a subject synthetic target protector nucleic acid, or a subject competitive inhibitor nucleic acid); and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyethylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl, KCl, MgSO₄, etc.

A subject pharmaceutical formulation can include a subject antisense nucleic acid in an amount of from about 0.001% to about 90% (w/w). A subject pharmaceutical formulation can include a subject target protector nucleic acid in an amount of from about 0.001% to about 90% (w/w). A subject pharmaceutical formulation can include a subject competitive inhibitor nucleic acid in an amount of from about 0.001% to about 90% (w/w). A subject pharmaceutical formulation can include a subject nucleic acid (e.g., a recombinant vector) that comprises a nucleotide sequence encoding a subject antisense nucleic acid, a subject synthetic target protector nucleic acid, or a subject competitive inhibitor nucleic acid, in an amount of from about 0.001% to about 90% (w/w). In the description of formulations, below, “subject nucleic acid” will be understood to include a subject antisense nucleic acid; a subject synthetic target protector nucleic acid; a subject competitive inhibitor nucleic acid; and a subject nucleic acid (e.g., a recombinant vector) that comprises a nucleotide sequence encoding a subject antisense nucleic acid, a subject synthetic target protector nucleic acid, or a subject competitive inhibitor nucleic acid. For example, in some embodiments, a subject formulation comprises a subject antisense nucleic acid. In other embodiments, a subject formulation comprises a subject target protector nucleic acid. In other embodiments, a subject formulation comprises a subject competitive inhibitor nucleic acid. In other embodiments, a subject formulation comprises a subject nucleic acid (e.g., a recombinant vector) that comprises a nucleotide sequence encoding a subject antisense nucleic acid, a subject synthetic target protector nucleic acid, or a subject competitive inhibitor nucleic acid.

A subject nucleic acid can be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption.

A subject nucleic acid can encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. For example, prodrug versions a subject nucleic acid can be prepared as SATE ((S acetyl-2-thioethyl) phosphate) derivatives according to the methods disclosed in WO 93/24510, WO 94/26764, and U.S. Pat. No. 5,770,713.

The term “pharmaceutically acceptable salts” refers to physiologically and pharmaceutically acceptable salts of a subject nucleic acid: i.e., salts that retain the desired biological activity of the parent compound and do not impart undesired toxicological effects thereto. For polynucleotides, suitable examples of pharmaceutically acceptable salts and their uses are further described in U.S. Pat. No. 6,287,860, which is incorporated herein by reference in its entirety.

The present disclosure also includes compositions and formulations, including pharmaceutical compositions and formulations, which include one or more of a subject nucleic acid. A subject composition can be administered in a number of ways depending upon whether local or systemic treatment is desired and upon the area to be treated. Administration can be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral, or parenteral. Parenteral administration includes, but is not limited to, intravenous, intraarterial, subcutaneous, intraperitoneal, or intramuscular injection or infusion; or intracranial, e.g., intrathecal or intraventricular, administration. Nucleic acids with at least one 2′-O-methoxyethyl modification can be used for oral administration. Compositions and formulations for topical administration can include transdermal patches, ointments, lotions, creams, gels, drops, suppositories, sprays, liquids, and powders. Conventional pharmaceutical carriers, aqueous, powder or oily bases, thickeners and the like may be necessary or desirable.

A subject formulation, which may conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

A subject composition can be formulated into any of many possible dosage forms such as, but not limited to, tablets, capsules, gel capsules, liquid syrups, soft gels, suppositories, and enemas. A subject composition can also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

A subject composition n may include solutions, emulsions, foams and liposome-containing formulations. A subject composition or formulation can omprise one or more penetration enhancers, carriers, excipients, or other active or inactive ingredients.

Emulsions are typically heterogeneous systems of one liquid dispersed in another in the form of droplets, which can exceed 0.1 μm in diameter. Emulsions may contain additional components in addition to the dispersed phases, and the active agent (e.g., antisense polynucleotides; target protector polynucleotides; competitive inhibitor polynucleotides; recombinant vector polynucleotides) which can be present as a solution in the aqueous phase, the oily phase, or as a separate phase. Microemulsions are also suitable. Emulsions and their uses are well known in the art and are further described in U.S. Pat. No. 6,287,860.

A subject formulation can be a liposomal formulation. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes that can interact with negatively charged nucleic acid molecules to form a stable complex. Liposomes that are pH sensitive or negatively charged are believed to entrap nucleic acid rather than complex with it. Both cationic and noncationic liposomes can be used to deliver a subject antisense nucleic acid.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein by reference in its entirety.

The formulations and compositions of the present disclosure may also include surfactants. The use of surfactants in drug products, formulations and in emulsions is well known in the art. Surfactants and their uses are further described in U.S. Pat. No. 6,287,860.

In one embodiment, various penetration enhancers are included, to effect the efficient delivery of nucleic acids, e.g., a subject antisense polynucleotide or a subject target protector nucleic acid. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein by reference in its entirety.

A subject nucleic acid can be conjugated to poly(L-lysine) to increase cell penetration. Such conjugates are described by Lemaitre et al., Proc. Natl. Acad. Sci. USA, 84, 648-652 (1987). The procedure requires that the 3′-terminal nucleotide be a ribonucleotide. The resulting aldehyde groups are then randomly coupled to the epsilon-amino groups of lysine residues of poly(L-lysine) by Schiff base formation, and then reduced with sodium cyanoborohydride. This procedure converts the 3′-terminal ribose ring into a morpholine structure antisense oligomer.

One of skill in the art will recognize that formulations are routinely designed according to their intended use and/or route of administration.

Suitable formulations for topical administration include those in which a subject nucleic acid is in admixture with a topical delivery agent such as lipids, liposomes, fatty acids, fatty acid esters, steroids, chelating agents and surfactants. Suitable lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, a subject nucleic acid can be encapsulated within liposomes or may form complexes thereto, in particular to cationic liposomes. Alternatively, a subject nucleic acid can be complexed to lipids, in particular to cationic lipids. Suitable fatty acids and esters, pharmaceutically acceptable salts thereof, and their uses are further described in U.S. Pat. No. 6,287,860.

Compositions and formulations for oral administration include powders or granules, microparticulates, nanoparticulates, suspensions or solutions in water or non-aqueous media, capsules, gel capsules, sachets, tablets, or minitablets. Thickeners, flavoring agents, diluents, emulsifiers, dispersing aids or binders may be desirable. Suitable oral formulations include those in which a subject antisense nucleic acid is administered in conjunction with one or more penetration enhancers, surfactants, and chelators. Suitable surfactants include, but are not limited to, fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. An exemplary suitable combination is the sodium salt of lauric acid, capric acid, and UDCA. Further penetration enhancers include, but are not limited to, polyoxyethylene-9-lauryl ether, and polyoxyethylene-20-cetyl ether. Suitable penetration enhancers also include propylene glycol, dimethylsulfoxide, triethanoiamine, N,N-dimethylacetamide, N,N-dimethylformamide, 2-pyrrolidone and derivatives thereof, tetrahydrofurfuryl alcohol, and AZONE™.

A subject nucleic acid can be delivered orally, in granular form including sprayed dried particles, or complexed to form micro or nanoparticles. Nucleic acid complexing agents and their uses are further described in U.S. Pat. No. 6,287,860.

Compositions and formulations for parenteral, intrathecal, or intraventricular administration may include sterile aqueous solutions which may also contain buffers, diluents and other suitable additives such as, but not limited to, penetration enhancers, carrier compounds and other pharmaceutically acceptable carriers or excipients.

### Delivery and Routes of Administration

A subject nucleic acid (e.g., a subject antisense nucleic acid; a subject synthetic target protector nucleic acid; a subject competitive inhibitor nucleic acid; a subject nucleic acid (e.g., a recombinant vector) comprising a nucleotide sequence encoding a subject antisense nucleic acid, a subject synthetic target protector nucleic acid, or a subject competitive inhibitor nucleic acid) can be administered by any suitable means. One skilled in the art will appreciate that many suitable methods of administering a subject nucleic acid (e.g., a subject antisense nucleic acid; a subject synthetic target protector nucleic acid; a subject competitive inhibitor nucleic acid; a subject nucleic acid (e.g., a recombinant vector) comprising a nucleotide sequence encoding a subject antisense nucleic acid, a subject synthetic target protector nucleic acid, or a subject competitive inhibitor nucleic acid) to a host in the context of the present disclosure, in particular a human, are available, and although more than one route may be used to administer a particular subject nucleic acid, a particular route of administration may provide a more immediate and more effective reaction than another route. In the following description of delivery and routes of administration, a “subject nucleic acid” will be understood to include a subject antisense nucleic acid and a subject synthetic target protector nucleic acid.

Suitable routes of administration include enteral and parenteral routes. Administration can be via a local or a systemic route of administration. A subject nucleic acid (e.g., a subject antisense nucleic acid; a subject synthetic target protector nucleic acid; a subject competitive inhibitor nucleic acid; a subject nucleic acid (e.g., a recombinant vector) comprising a nucleotide sequence encoding a subject antisense nucleic acid, a subject synthetic target protector nucleic acid, or a subject competitive inhibitor nucleic acid) can be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes, but is not limited to, intravenous, intraarterial, subcutaneous, intraperitoneal, or intramuscular injection or infusion; and intracranial, e.g., intrathecal or intraventricular, administration. Peritumoral administration is also contemplated.

### Dosing

The formulation of therapeutic compositions and their subsequent administration (dosing) is within the skill of those in the art. Dosing is dependent on several criteria, including severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC50s found to be effective in vitro and in vivo animal models.

For example, a suitable dose of a subject nucleic acid (e.g., a subject antisense nucleic acid; a subject synthetic target protector nucleic acid; a subject competitive inhibitor nucleic acid; a subject nucleic acid (e.g., a recombinant vector) comprising a nucleotide sequence encoding a subject antisense nucleic acid, a subject synthetic target protector nucleic acid, or a subject competitive inhibitor nucleic acid) is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein a subject nucleic acid (e.g., a subject antisense nucleic acid; a subject synthetic target protector nucleic acid; a subject competitive inhibitor nucleic acid; a subject nucleic acid (e.g., a recombinant vector) comprising a nucleotide sequence encoding a subject antisense nucleic acid, a subject synthetic target protector nucleic acid, or a subject competitive inhibitor nucleic acid) is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

In some embodiments, multiple doses of a subject nucleic acid (e.g., a subject antisense nucleic acid; a subject synthetic target protector nucleic acid; a subject competitive inhibitor nucleic acid; a subject nucleic acid (e.g., a recombinant vector) comprising a nucleotide sequence encoding a subject antisense nucleic acid, a subject synthetic target protector nucleic acid, or a subject competitive inhibitor nucleic acid) are administered. The frequency of administration of an active agent (a subject nucleic acid) can vary depending on any of a variety of factors, e.g., severity of the symptoms, etc. For example, in some embodiments, a subject nucleic acid (e.g., a subject antisense nucleic acid; a subject synthetic target protector nucleic acid; a subject competitive inhibitor nucleic acid; a subject nucleic acid (e.g., a recombinant vector) comprising a nucleotide sequence encoding a subject antisense nucleic acid, a subject synthetic target protector nucleic acid, or a subject competitive inhibitor nucleic acid) is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of an active agent (e.g., a subject antisense nucleic acid; a subject synthetic target protector nucleic acid; a subject competitive inhibitor nucleic acid; a subject nucleic acid (e.g., a recombinant vector) comprising a nucleotide sequence encoding a subject antisense nucleic acid, a subject synthetic target protector nucleic acid, or a subject competitive inhibitor nucleic acid), e.g., the period of time over which an active agent is administered, can vary, depending on any of a variety of factors, e.g., patient response, etc. For example, an active agent can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Methods of Inhibiting Angiogenesis

The present disclosure provides methods of inhibiting angiogenesis in an individual in need thereof, where the methods generally involve administering to the individual an effective amount of an agent that reduces the level and/or activity of a miR-126 nucleic acid in an endothelial cell in the individual, or administering to the individual an effective amount of a subject synthetic target protector nucleic acid.

Agents that reduce the level and/or activity of an miR-126 nucleic acid in an endothelial cell include antisense nucleic acids (e.g., a miR-126 antisense nucleic acid, as described above); nucleic acids comprising nucleotide sequences encoding a subject miR-126 antisense nucleic acid; a subject competitive inhibitor nucleic acid; a nucleic acid comprising a nucleotide sequence encoding a subject competitive inhibitor nucleic acid; and the like.

Whether angiogenesis is reduced can be determined using any known method. Methods of determining an effect of an agent (e.g., a subject nucleic acid, e.g., a subject antisense nucleic acid; a subject synthetic target protector nucleic acid; a subject competitive inhibitor nucleic acid; a nucleic acid comprising a nucleotide sequence encoding a subject antisense nucleic acid, a subject synthetic target protector nucleic acid, or a subject competitive inhibitor nucleic acid) on angiogenesis are known in the art and include, but are not limited to, inhibition of neovascularization into implants impregnated with an angiogenic factor; inhibition of blood vessel growth in the cornea or anterior eye chamber; inhibition of endothelial tube formation in vitro; the chick chorioallantoic membrane assay; the hamster cheek pouch assay; the polyvinyl alcohol sponge disk assay. Such assays are well known in the art and have been described in numerous publications, including, e.g., Auerbach et al. ((1991) Pharmac. Ther. 51:1-11), and references cited therein.

The invention further provides methods for treating a condition or disorder associated with or resulting from pathological angiogenesis. In the context of cancer therapy, a reduction in angiogenesis according to the methods of the invention effects a reduction in tumor size; and a reduction in tumor metastasis. Whether a reduction in tumor size is achieved can be determined, e.g., by measuring the size of the tumor, using standard imaging techniques. Whether metastasis is reduced can be determined using any known method. Methods to assess the effect of an agent on tumor size are well known, and include imaging techniques such as computerized tomography and magnetic resonance imaging.

Any condition or disorder that is associated with or that results from pathological angiogenesis, or that is facilitated by neovascularization (e.g., a tumor that is dependent upon neovascularization), is amenable to treatment with an agent that reduces the level of an miR-126 nucleic acid in an endothelial cell, so as to inhibit angiogenesis.

Conditions and disorders amenable to treatment include, but are not limited to, cancer; atherosclerosis; proliferative retinopathies such as retinopathy of prematurity, diabetic retinopathy, age-related maculopathy, retrolental fibroplasia; excessive fibrovascular proliferation as seen with chronic arthritis; psoriasis; and vascular malformations such as hemangiomas, and the like.

The instant methods are useful in the treatment of both primary and metastatic solid tumors, including carcinomas, sarcomas, leukemias, and lymphomas. Of particular interest is the treatment of tumors occurring at a site of angiogenesis. Thus, the methods are useful in the treatment of any neoplasm, including, but not limited to, carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). The instant methods are also useful for treating solid tumors arising from hematopoietic malignancies such as leukemias (i.e. chloromas, plasmacytomas and the plaques and tumors of mycosis fungoides and cutaneous T-cell lymphoma/leukemia) as well as in the treatment of lymphomas (both Hodgkin's and non-Hodgkin's lymphomas). In addition, the instant methods are useful for reducing metastases from the tumors described above either when used alone or in combination with radiotherapy and/or other chemotherapeutic agents.

Other conditions and disorders amenable to treatment using the methods of the instant invention include autoimmune diseases such as rheumatoid, immune and degenerative arthritis; various ocular diseases such as diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, and other abnormal neovascularization conditions of the eye; skin diseases such as psoriasis; blood vessel diseases such as hemangiomas, and capillary proliferation within atherosclerotic plaques; Osler-Webber Syndrome; plaque neovascularization; telangiectasia; hemophiliac joints; angiofibroma; and excessive wound granulation (keloids).

In order to accomplish reduction of angiogenesis in vivo (e.g., as in the context of treating pathological angiogenesis), an agent that reduces the level of an miR-126 nucleic acid in an endothelial cell, or a subject synthetic target protector nucleic acid, will be administered in any suitable manner, typically with pharmaceutically acceptable carriers. One skilled in the art will readily appreciate that the a variety of suitable methods of administering an active agent (e.g., an agent that reduces the level and/or activity of an miR-126 nucleic acid in an endothelial cell; a subject synthetic target protector nucleic acid) in the context of the present disclosure to a subject are available, and, although more than one route can be used to administer a particular compound, a particular route can provide a more immediate, more effective, and/or associated with fewer side effects than another route. In general, an active agent can be administered according to the method of the invention by, for example, a parenteral, intratumoral, peritumoral, intravenous, intra-arterial, inter-pericardial, intramuscular, intraperitoneal, transdermal, transcutaneous, subdermal, intradermal, or intrapulmonary route.

In some embodiments, an active agent (e.g., an agent that reduces the level and/or activity of an miR-126 nucleic acid in an endothelial cell; a subject synthetic target protector nucleic acid) will be delivered locally. Local administration can be accomplished by, for example, direct injection (e.g., intramuscular injection, intratumoral injection) at the desired treatment site, by introduction of the active agent formulation intravenously at a site near a desired treatment site (e.g., into a vessel or capillary that feeds a treatment site), by intra-arterial introduction, by introduction (e.g., by injection or other method of implantation) of an active agent formulation in a biocompatible gel or capsule within or adjacent a treatment site, by injection directly into muscle or other tissue in which a decrease in pathological angiogenesis is desired, etc.

In another embodiment of interest, the active agent formulation is delivered in the form of a biocompatible gel, which can be implanted (e.g., by injection into or adjacent a treatment site, by extrusion into or adjacent a tissue to be treated, etc.). Gel formulations comprising an active agent can be designed to facilitate local release of the active agent for a sustained period (e.g., over a period of hours, days, weeks, etc.). The gel can be injected into or near a treatment site, e.g., using a needle or other delivery device.

The desirable extent of reduction of pathological angiogenesis will depend on the particular condition or disease being treated, as well as the stability of the patient and possible side-effects.

### Combination Therapy

A subject method of decreasing angiogenesis (e.g., to treat a disorder associated with pathological angiogenesis) can involve administering an agent that decreases the level and/or activity of miR-126 nucleic acid in an endothelial cell in an individual, and can further involve administering at least a second therapeutic agent. A subject method of decreasing angiogenesis (e.g., to treat a disorder associated with pathological angiogenesis) can involve administering a subject synthetic target protector nucleic acid, and can further involve administering at least a second therapeutic agent. Suitable second therapeutic agents include agents that reduce angiogenesis; anti-cancer chemotherapeutic agents; anti-inflammatory agents; etc.

An agent that decreases the level of miR-126 nucleic acid in an endothelial cell can be administered in combination therapy with one or more additional nucleic acids that modulate the level of a pro-angiogenic microRNA. Proangiogenic microRNAs include, e.g., miR-27b, miR-210, miR-130a, miR-296, and miR-378. An agent that reduces the level of a proangiogenic microRNA in an endothelial cell would be expected to reduce angiogenesis in the endothelial cell. Such agents include, e.g., antisense nucleic acids, antagomirs, competitive inhibitor nucleic acids, etc.

An agent that decreases the level of miR-126 nucleic acid in an endothelial cell can be administered in combination therapy with at least a second therapeutic agent, e.g. an agent that reduces angiogenesis. Agents that reduce angiogenesis include, e.g., a soluble vascular endothelial growth factor (VEGF) receptor; 2-ME (NSC-659853); PI-88 (D-mannose), O-6-O-phosphono-alpha-D-mannopyranosyl-(1-3)-O-alpha-D-manno-pyranosyl-(1-3)-O-alpha-D-mannopyranosyl-(1-3)-O-alpha-D-mannopyranosyl-(1- -2)-hydrogen sulphate); thalidomide (1H-isoindole-1,3 (2H)-dione, 2-(2,6-dioxo-3-piperidinyl)-); CDC-394; CC-5079; ENMD-0995 (S-3-amino-phthalidoglutarimide); AVE-8062A; vatalanib; SH-268; halofuginone hydrobromide; atiprimod dimaleate (2-azaspivo[4.5]decane-2-propanamine, N,N-diethyl-8,8-dipropyl, dimaleate); ATN-224; CHIR-258; combretastatin A-4 (phenol, 2-methoxy-5-[2-(3,4,5-trimethoxyphenyl]etheny-1]-, (Z)-); GCS-100LE, or an analogue or derivative thereof; 2-methoxyestradiol; A6; ABT-510; ABX-IL8, actimid, Ad5FGF-4, AG3340, alpha5beta1 integrin antibody, AMG001, anecortave acetate, angiocol, angiogenix, angiostatin, angiozyme, antiangiogenic antithrombin 3, anti-VEGF, anti-VEGF monoclonal antibody (mAb), aplidine, aptosyn, ATN-161, avastin, AVE8062A, Bay 12-9566, benefin, BioBypass CAD, MS275291, CAI, carboxymidotriazole, CC 4047, CC 5013, CC7085, CDC801, Celebrex, CEP-7055, CGP-41251/PKC412, cilengitide, CM101, col-3, combretastatin, combretastatin A4P, CP-547, 632, CP-564, 959, Del-1, dexrazoxane, didemnin B, DMXAA, EMD 121974, endostatin, FGF (AGENT 3), flavopiridol, GBC-100, genistein concentrated polysaccharide, green tea extract, HIF-1 alpha, human chorio-gonadotrophin, IM862, INGN 201, interferon alpha-2a, interleukin-12, Iressa, ISV-120, LY317615, LY-333531, mAb huJ591-DOTA-90 Yttrium, marimastat, Medi-522, metaret, neoretna, neovastat, NM-3, NPe6, NVIFGF, octreotide, oltipraz, paclitaxel, pegaptanib sodium, penicillamine, pentosan polysulphate, prinomastat, PSK, psorvastat, PTK787/ZK222584, ranibizumab, razoxane, replistatatin, revimid, RhuMab, Ro317453, squalamine, SU101, SU11248, SU5416, SU6668, tamoxifen, tecogalan sodium, temptostatin, tetrathiomol, tetrathiomolybdate, thalomid, TNP-470, UCN-01, VEGF, VEGF trap, Vioxx, vitaxin, vitaxin-2, ZD6126, ZD6474, angiostatin (plasminogen fragment), a tissue inhibitor of matrix metalloproteinase (TIMP), antiangiogenic antithrombin III, pigment epithelial-derived factor (PEDF), canstatin, placental ribonuclease inhibitor, cartilage-derived inhibitor (CDI), plasminogen activator inhibitor, CD59 complement fragment, platelet factor-4, endostatin (collagen XVIII fragment), prolactin 16 kD fragment, fibronectin fragment, proliferin-related protein, gro-beta, a retinoid, a heparinase, tetrahydrocortisol-S, heparin hexasaccharide fragment, thrombospondin-1, human chorionic gonadotropin, transforming growth factor-beta, interferon alpha, interferon beta, or interferon gamma, tumistatin, interferon inducible protein, vasculostatin, interleukin-12, vasostatin (calreticulin fragment), kringle 5 (plasminogen fragment), angioarrestin, or 2-methoxyestradiol.

Angiogenesis inhibitors also include antagonists of angiogenin, placental growth factor, angiopoietin-1, platelet-derived endothelial cell growth factor, Del-1, platelet-derived growth factor-BB, aFGF, bFGF, pleiotrophin, follistatin, proliferin, granulocyte colony-stimulating factor, transforming growth factor-alpha, hepatocyte growth factor, transforming growth factor-beta, interleukin-8, tumor necrosis factor-alpha, and vascular endothelial growth factor. Angiogenesis inhibitors further include ABT-510, ABX-IL8 (Abgenix), actimid, Ad5FGF-4 (Collateral Therapeutics), AG3340 (Agouron Pharmaceuticals Inc. LaJolla, Calif.), α5β1 integrin antibody, AMG001 (AnGes/Daichi Pharmaceuticals), anecortave acetate (Retaane, Alcon), angiocol, angiogenix (Endovasc Ltd), angiostatin (EntreMed), angiozyme, antiangiogenic antithrombin 3 (Genzyme Molecular Oncology), anti-VEGF (Genentech), anti-VEGF mAb, aplidine, aptosyn, ATN-161, avastin (bevacizumab), AVE8062A, Bay 12-9566 (Bayer Corp. West Haven, Conn.), benefin, BioBypass CAD (VEGF-121) (GenVec), MS275291, CAI (carboxy-amido imidazole), carboxymidotriazole, CC 4047 (Celgene), CC 5013 (Celgene), CC7085, CDC 801 (Celgene), Celebrex (Celecoxib), CEP-7055, CGP-41251/PKC412, cilengitide, CM 101 (Carbomed Brentwood, Term.), col-3 (CollaGenex Pharmaceuticals Inc. Newton, Pa.), combretastatin, combretastatin A4P (Oxigene/Bristol-Myers Squibb), CP-547, 632, CP-564, 959, Del-1 (VLTS-589) (Valentis), dexrazoxane, didemnin B, DMXAA, EMD 121974, endostatin (EntreMed), FGF (AGENT 3) (Berlex (Krannert Institute of Cardiology)), flavopiridol, GBC-100, genistein concentrated polysaccharide, IM862 (Cytran), INGN 201, interferon alpha-2a, interleukin-12, Iressa, ISV-120 (Batimastat), LY317615, LY-333531 (Eli Lilly and Company), mAb huJ591-DOTA-90 Yttrium (90Y), marimastat (British Biotech Inc. Annapolis, Md.), Medi-522, metaret (suramin), neoretna, neovastat (AEtema Laboratories), NM-3, NPe6, NV1FGF (Gencel/Aventis), octreotide, oltipraz, paclitaxel (e.g., taxol, docetaxel, or paxene), pegaptanib sodium (Eyetech), penicillamine, pentosan polysulphate, PI-88, prinomastat (Agouron Pharmaceuticals), PSK, psorvastat, PTK787/ZK222584, ranibizumab (Lucentis, Genentech), razoxane, replistatatin (Platelet factor-4), revimid, RhuMab, Ro317453, squalamine (Magainin Pharmaceuticals, Inc. Plymouth Meeting, Pa.), SU101 (Sugen Inc. Redwood City, Calif.), SU11248, SU5416 (Sugen), SU6668 (Sugen), tamoxifen, tecogalan sodium, temptostatin, tetrathiomol, and tetrathiomolybdate.

An agent that decreases the level and/or activity of miR-126 nucleic acid in an endothelial cell can be administered in combination therapy with one or more chemotherapeutic agents for treating cancer. Chemotherapeutic agents for treating cancer include non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

### Methods of Increasing Angiogenesis

The present disclosure provides methods of increasing angiogenesis in an individual in need thereof, the methods generally involving administering to the individual an effective amount of an agent that increases the level of a miR-126 nucleic acid in an endothelial cell in the individual. Increasing angiogenesis can provide for therapeutic angiogenesis. An agent that increases the level of a miR-126 nucleic acid in an endothelial cell in an individual can stimulate therapeutic angiogenesis in the individual. Thus, in some embodiments, the instant invention provides a method of increasing or stimulating therapeutic angiogenesis in an individual, where increasing or stimulating therapeutic angiogenesis can treat a disorder that is amenable to treatment by stimulating or increasing angiogenesis.

An effective amount of an active agent (e.g., an agent that increases the level of a miR-126 nucleic acid in an endothelial cell) increases angiogenesis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 2-fold, at least about 5-fold, at least about 10-fold, or more, when compared to an untreated (e.g., a placebo-treated) control. Stimulation of angiogenesis is useful to treat a variety of conditions that would benefit from stimulation of angiogenesis, stimulation of vasculogenesis, increased blood flow, and/or increased vascularity.

An agent that increases the level of a miR-126 nucleic acid in an endothelial cell in an individual includes a recombinant nucleic acid comprising a nucleotide sequence encoding a miR-126 nucleic acid. A recombinant nucleic acid can be an expression vector that comprises a nucleotide sequence encoding a miR-126 nucleic acid.

A miR-126-encoding nucleotide sequence can be included in an expression vector, resulting in a recombinant expression vector comprising a nucleotide sequence encoding a miR-126 nucleic acid. Expression vectors generally have convenient restriction sites located near the promoter sequence to provide for the insertion of a nucleic acid of interest (e.g., a nucleic acid comprising a nucleotide sequence encoding a miR-126 nucleic acid). A selectable marker operative in the expression host may be present.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641648, 1999; Ali et al., Hum Mol Genet. 5:591594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; a lentivirus; a human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Suitable eukaryotic vectors include, for example, bovine papilloma virus-based vectors, Epstein-Barr virus-based vectors, vaccinia virus-based vectors, SV40, 2-micron circle, pcDNA3.1, pcDNA3.1/GS, pYES2/GS, pMT, p IND, pIND(Sp1), pVgRXR (Invitrogen), and the like, or their derivatives. Such vectors are well known in the art (Botstein et al., Miami Wntr. SyTnp. 19:265-274, 1982; Broach, In: “The Molecular Biology of the Yeast Saccharomyces: Life Cycle and Inheritance”, Cold Spring Harbor Laboratory, Cold Spring Harbor, N.Y., p. 445-470, 1981; Broach, Cell 28:203-204, 1982; Dilon et al., J. Clin. Hematol. Onco1.10:39-48, 1980; Maniatis, In: Cell Biology: A Comprehensive Treatise, Vol. 3, Gene Sequence Expression, Academic Press, NY, pp. 563-608,1980.

The recombinant vector can include one or more coding regions that encode a polypeptide (a “selectable marker”) that allow for selection of the recombinant vector in a genetically modified host cell comprising the recombinant vector. Suitable selectable markers include those providing antibiotic resistance; e.g., blasticidin resistance, neomycin resistance. Several selectable marker genes that are useful include the hygromycin B resistance gene (encoding aminoglycoside phosphotranferase (APH)) that allows selection in mammalian cells by conferring resistance to hygromycin; the neomycin phosphotranferase gene (encoding neomycin phosphotransferase) that allows selection in mammalian cells by conferring resistance to G418; and the like.

In some embodiments, the recombinant vector integrates into the genome of the host cell (e.g., an endothelial cell); in other embodiments, the recombinant vector is maintained extrachromosomally in the host cell comprising the recombinant vector. A host cell (e.g., an endothelial cell) comprising a recombinant vector comprising a nucleotide sequence encoding a miR-126 nucleic acid is a “genetically modified” host cell.

A miR-126-encoding nucleotide sequence is operably linked to one or more transcriptional control elements, e.g., a promoter. Non-limiting examples of suitable eukaryotic promoters (promoters functional in a eukaryotic cell) include cytomegalovirus (CMV) immediate early, herpes simplex virus (HSV) thymidine kinase, early and late SV40, long terminal repeats (LTRs) from retrovirus, and mouse metallothionein-I. In some embodiments, the promoter is a constitutive promoter. Non-limiting examples of constitutive promoters include: ubiquitin promoter, CMV promoter, JeT promoter (U.S. Pat. No. 6,555,674), SV40 promoter, Elongation Factor 1 alpha promoter (EF1-alpha), RSV, and Mo-MLV-LTR. In some embodiments, the promoter is an inducible promoter. Non-limiting examples of inducible/repressible promoters include: Tet-On, Tet-Off, Rapamycin-inducible promoter, and Mx1. Selection of the appropriate vector and promoter is well within the level of ordinary skill in the art.

In some embodiments, the promoter is an endothelial cell-specific promoter. Endothelial cell-specific promoters include, e.g., a preproendothelin-1 (PPE-1) promoter, a PPE-1-3× promoter, a TIE-1 promoter, a TIE-2 promoter, an endoglin promoter, a von Willebrand factor (vWF) promoter, a KDR/flk-1 promoter, an endothelin-1 promoter, a FLT-1 promoter, an Egr-1 promoter, an ICAM-1 promoter, a VCAM-1 promoter, a PECAM-1 promoter, and an aortic carboxypeptidase-like protein (ACLP) promoter. Endothelial cell-specific promoters are known in the art; see, e.g., U.S. Pat. No. 5,888,765 (KDR/flk-1 promoter); U.S. Pat. No. 6,200,751 (endothelin-1 promoter); Cowan et al. (1998) J. Biol. Chem. 273:11737 (ICAM-2 promoter); Fadel et al. (1998) Biochem. J. 330:335 (TIE-2 promoter); and Dai et al. (2004) J. Virol. 78:6209 (synthetic EC-specific promoters); U.S. Pat. No. 7,067,649 (PPE-1 promoter); Varda-Bloom et al. (2001) Gene Ther. 8:819 (PPE-1 promoter); Velasco et al. (2001) Gene Ther. 8:897 (endoglin promoter; U.S. Pat. No. 6,103,527 (endoglin promoter); Ozaki et al. (1996) Hum. Gene Ther. 20:1483 (vWF promoter); and WO 2006/051545. Also suitable for use are a vascular-endothelial-cadherin (VE-Cadherin) promoter (Prandini et al, Oncogene, 2005, Apr. 21; 24(18):2992-3001); a MEF2C promoter (de Val et al, Cell, 2008, Dec. 12; 135(6); and an endothelial nitric oxide synthase (eNOS) promoter (Guillot et al, J. Clin Invest, 1999, March; 103(6):799-805). Also suitable are inducible versions of an endothelial cell-specific promoter; e.g., TIE-2 (Forde et al, Genesis, 2002, August; 33(4):191-7 and Deutsch et al, Exp Cell Res, 2008, Apr. 1; 314(6):1202-16) and VE-Cadherin (used in Hellstrom et al, Nature, 2007, Feb. 15; 445(7129):776-80)).

Examples of conditions and diseases amenable to treatment according to a subject method related to increasing angiogenesis include any condition associated with an obstruction of a blood vessel, e.g., obstruction of an artery, vein, or of a capillary system. Specific examples of such conditions or disease include, but are not necessarily limited to, coronary occlusive disease, carotid occlusive disease, arterial occlusive disease, peripheral arterial disease, atherosclerosis, myointimal hyperplasia (e.g., due to vascular surgery or balloon angioplasty or vascular stenting), thromboangiitis obliterans, thrombotic disorders, vasculitis, and the like. Examples of conditions or diseases that can be reduced using the methods of the invention include, but are not necessarily limited to, heart attack (myocardial infarction) or other vascular death, stroke, death or loss of limbs associated with decreased blood flow, and the like.

Other forms of therapeutic angiogenesis include, but are not necessarily limited to, the use of an active agent that increases the level of a miR-126 nucleic acid in an endothelial cell to accelerate healing of wounds or ulcers (e.g., as a result of physical injury or disease, e.g., cutaneous ulcers, diabetic ulcers, ulcerative colitis, and the like); to improve the vascularization of skin grafts or reattached limbs so as to preserve their function and viability; to improve the healing of surgical anastomoses (e.g., as in re-connecting portions of the bowel after gastrointestinal surgery); and to improve the growth of skin or hair.

In order to accomplish stimulation of angiogenesis in vivo (e.g., as in the context of therapeutic angiogenesis), an active agent that increases the level of a miR-126 nucleic acid in an endothelial cell can be administered in any suitable manner, preferably with pharmaceutically acceptable carriers. One skilled in the art will readily appreciate that the a variety of suitable methods of administering an active agent in the context of the present disclosure to a subject are available, and, although more than one route can be used to administer a particular compound, a particular route can provide a more immediate, more effective, and/or associated with fewer side effects than another route. In general, an active agent is administered according to the method of the invention by, for example, a parenteral, intravenous, intra-arterial, inter-pericardial, intramuscular, intraperitoneal, transdermal, transcutaneous, subdermal, intradermal, or intrapulmonary route.

In some embodiments, an active agent will be delivered locally. Local administration can be accomplished by, for example, direct injection (e.g., intramuscular injection) at the desired treatment site, by introduction of the active agent formulation intravenously at a site near a desired treatment site (e.g., into a vessel or capillary that feeds a treatment site), by intra-arterial or intra-pericardial introduction, by introduction (e.g., by injection or other method of implantation) of an active agent formulation in a biocompatible gel or capsule within or adjacent a treatment site, by injection directly into muscle or other tissue in which increased blood flow and/or increased vascularity is desired, by rectal introduction of the formulation (e.g., in the form of a suppository to, for example, facilitate vascularization of a surgically created anastomosis after resection of a piece of the bowel), etc.

In some embodiments it may be desirable to deliver the active agent directly to the wall of a vessel. One exemplary method of vessel wall administration involves the use of a drug delivery catheter, particularly a drug delivery catheter comprising an inflatable balloon that can facilitate delivery to a vessel wall. Thus, in one embodiment the method of the invention comprises delivery of an active agent to a vessel wall by inflating a balloon catheter, wherein the balloon comprises an active agent formulation covering a substantial portion of the balloon. The active agent formulation is held in place against the vessel wall, promoting adsorption through the vessel wall. In one example, the catheter is a perfusion balloon catheter, which allows perfusion of blood through the catheter while holding the active agent against the vessel walls for longer adsorption times. Examples of catheters suitable for active agent application include drug delivery catheters disclosed in U.S. Pat. Nos. 5,558,642; U.S. Pat. Nos. 5,554,119; 5,591,129; and the like.

In another embodiment of interest, the active agent formulation is delivered in the form of a biocompatible gel, which can be implanted (e.g., by injection into or adjacent a treatment site, by extrusion into or adjacent a tissue to be treated, etc.). Gel formulations comprising an active agent can be designed to facilitate local release of the active agent for a sustained period (e.g., over a period of hours, days, weeks, etc.). The gel can be injected into or near a treatment site, e.g., using a needle or other delivery device. In one embodiment, the gel is placed into or on an instrument which is inserted into the tissue and then slowly withdrawn to leave a track of gel, resulting in stimulation of angiogenesis along the path made by the instrument. This latter method of delivery may be particularly desirable for, for the purpose of directing course of the biobypass.

In other embodiments it may be desirable to deliver the active agent formulation topically, e.g., for localized delivery, e.g., to facilitate wound healing. Topical application can be accomplished by use of a biocompatible gel, which may be provided in the form of a patch, or by use of a cream, foam, and the like. Several gels, patches, creams, foams, and the like appropriate for application to wounds can be modified for delivery of active agent formulations according to the invention (see, e.g., U.S. Pat. Nos. 5,853,749; 5,844,013; 5,804,213; 5,770,229; and the like). In general, topical administration is accomplished using a carrier such as a hydrophilic colloid or other material that provides a moist environment. Alternatively, for the purpose of wound healing the active agent could be supplied, with or without other angiogenic agents in a gel or cream then could be applied to the wound. An example of such an application would be as a sodium carboxymethylcellulose-based topical gel with a low bioburden containing the active agent and other active ingredients together with preservatives and stabilizers.

In other embodiments, the active agent formulation is delivered locally or systemically, e.g., locally, using a transdermal patch. Several transdermal patches are well known in the art for systemic delivery of nicotine to facilitate smoking cessation, and such patches may be modified to provide for delivery of an amount of active agent effective to stimulate angiogenesis according to the invention (see, e.g., U.S. Pat. Nos. 4,920,989; and 4,943,435, NICOTROL™ patch, and the like).

In other methods of delivery, the active agent can be administered using iontophoretic techniques. Methods and compositions for use in iontophoresis are well known in the art (see, e.g., U.S. Pat. Nos. 5,415,629; 5,899,876; 5,807,306; and the like).

The desirable extent of angiogenesis will depend on the particular condition or disease being treated, as well as the stability of the patient and possible side-effects. In proper doses and with suitable administration, the present disclosure provides for a wide range of development of blood vessels, e.g., from little development to essentially full development.

### Combination Therapy

A subject method of increasing angiogenesis (e.g., to treat a disorder amenable to treatment by increasing angiogenesis) can involve administering an agent that increases the level of miR-126 nucleic acid in an endothelial cell in an individual, and can further involve administering at least a second therapeutic agent. Suitable second therapeutic agents include agents (including polypeptide agents and non-polypeptide agents) that increase angiogenesis; wound-healing agents; additional proangiogenic microRNAs; etc.

An agent that increases the level of a miR-126 nucleic acid in an endothelial cell can be administered in combination therapy with at least one additional agent that increases the level of a proangiogenic microRNA (other than miR-126) in an endothelial cell. Proangiogenic microRNAs include, e.g., miR-27b, miR-210, miR-130a, miR-296, and miR-378.

An agent that increases the level of a miR-126 nucleic acid in an endothelial cell can be administered in combination therapy with at least one angiogenic polypeptide. Suitable angiogenic polypeptides include, but are not limited to, VEGF polypeptides, including VEGF₁₂₁, VEGF₁₆₅, VEGF-C, VEGF-2, etc.; transforming growth factor-beta; basic fibroblast growth factor; glioma-derived growth factor; angiogenin; angiogenin-2; and the like. The amino acid sequences of various angiogenic agents are publicly available, e.g., in public databases such as GenBank; journal articles; patents and published patent applications; and the like. For example, amino acid sequences of VEGF polypeptides are disclosed in U.S. Pat. Nos. 5,194,596, 5,332,671, 5,240,848, 6,475,796, 6,485,942, and 6,057,428; amino acid sequences of VEGF-2 polypeptides are disclosed in U.S. Pat. Nos. 5,726,152 and 6,608,182; amino acid sequences of glioma-derived growth factors having angiogenic activity are disclosed in U.S. Pat. Nos. 5,338,840 and 5,532,343; amino acid sequences of angiogenin are found under GenBank Accession Nos. AAA72611, AAA51678, AAA02369, AAL67710, AAL67711, AAL67712, AAL67713, and AAL67714; etc.

### Subjects Suitable for Treatment

Individuals who are suitable for treatment with a subject method include individuals having a disorder that is amenable to treatment by increasing angiogenesis (in the case of a subject method of increasing angiogenesis); and individuals having a disorder that is amenable to treatment by decreasing angiogenesis (in the case of a subject method of decreasing angiogenesis).

### Methods of Decreasing Angiogenesis

Individuals who are suitable for treatment with a subject method for decreasing angiogenesis include individuals having a disorder associated with (e.g., resulting from) pathological angiogenesis. For example, individuals who are suitable for treatment with a subject method of decreasing angiogenesis include individuals who have a disorder such as cancer; atherosclerosis; an ocular disorder such as proliferative retinopathies such as retinopathy of prematurity, diabetic retinopathy, age-related maculopathy, retrolental fibroplasia; excessive fibrovascular proliferation as seen with chronic arthritis; psoriasis; and vascular malformations such as hemangiomas, and the like.

For example, individuals who are suitable for treatment with a subject method of decreasing angiogenesis include individuals who have any of the above-mentioned cancers; individuals who have cancer and in whom the cancer has metastasized; individuals who have undergone treatment for a cancer and who failed to respond; and individuals who have undergone treatment for a cancer, who initially responded, and who subsequently relapsed.

Individuals who are suitable for treatment with a subject method of decreasing angiogenesis include individuals having a disorder such as an autoimmune disease such as rheumatoid, immune and degenerative arthritis; an ocular disease such as diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, and other abnormal neovascularization conditions of the eye; a skin disease such as psoriasis; a blood vessel disease such as hemangiomas, and capillary proliferation within atherosclerotic plaques; Osler-Webber Syndrome; plaque neovascularization; telangiectasia; hemophiliac joints; angiofibroma; and excessive wound granulation (keloids).

### Methods of Increasing Angiogenesis

Individuals who are suitable for treatment with a subject method for increasing angiogenesis include individuals having a disorder such as a wound or an ulcer (e.g., as a result of physical injury or disease, e.g., cutaneous ulcers, diabetic ulcers, ulcerative colitis, and the like); an individual who is the recipient of a skin graft; an individual who has undergone limb reattachment; an individual who has undergone surgical anastomoses (e.g., as in re-connecting portions of the bowel after gastrointestinal surgery); etc.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Regulation of VEGF Signaling and Vascular Integrity by miR-126

### Experimental Procedures

### Cell Culture

Human umbilical vein endothelial cells (HUVECs) were purchased from ScienCell and cultured according to the manufacturer's recommendations. The HeLa cell line was purchased from the American Type Culture Collection (ATCC). E14 embryonic stem (ES) cells were cultured on gelatin and supplemented with maintenance medium (Glasgow MEM (Sigma) containing 10% fetal bovine serum (FBS) (HyClone), 1 mM 2-mercaptoethanol (Sigma), 2 mM L-glutamine (Gibco-BRL), 1 mM sodium pyruvate, 0.1 mM minimal essential medium containing nonessential amino acids, and leukemia inhibitory factor (LIF)-conditioned medium (1:1000)). Differentiation of ES cells into embryoid bodies (EBs) was performed by the hanging-drop method. Approximately 500 ES cells were suspended in 20 μL of differentiation medium (containing the same components as maintenance medium but with 20% FBS and no LIF) per well of a 96-well conical plate and left inverted for 2 days. Plates were then inverted right-side up, and new differentiation medium was added. Media were changed every 2 days of culture.

### Fluorescence-Activated Cell Sorting

Single-cell suspensions were generated by digesting EBs or mouse embryos with Accutase (Chemicon). Cells were resuspended in phosphate buffered saline (PBS) containing 1% bovine serum albumin (BSA) and labeled with fluorochrome-conjugated primary antibodies. For the separation of Flk1-positive cells from mouse EBs, phycoerytherin (PE)-conjugated anti-mouse Flk1 antibody (BD Pharmingen, Avas 12α, Cat. Number: 555308) was used. For the separation of CD31-positive cells from mouse EBs or embryos, fluorescein isothiocyanate (FITC)-conjugated anti-mouse CD31 antibody (BD Pharmingen, MEC 13.3, Cat. Number: 553372) was used. Antibodies were used at 5 μg/mL and were incubated with cells for 30 min at 4° C. with rotation, and cells were then washed with PBS-1% BSA. Cells were sorted in PBS-0.1% BSA with a fluorescence activated cell sorting (FACS) Diva flow cytometer and cell sorter (Becton Dickinson). For the sorting of endothelial cells from Tg(flk1:GFP)^{s843} zebrafish, embryos were digested with trypsin and sorted based on green fluorescent protein (GFP) fluorescence.

### Plasmids

For identifying mRNAs targeted by miR-126, portions of the 3′ untranslated region (3′UTR) (˜500 bp) of potential targets were polymerase chain reaction (PCR) amplified with XbaI linkers from human or zebrafish genomic DNA or cDNA and cloned into the XbaI site at the end of the luciferase open reading frame (ORF) of pGL3 control. Sequencing was used to determine the orientation of the inserts. To over-express miR-1 and miR-126 in cells, genomic regions of these genes, including some flanking DNA (˜500 bp total), were inserted into an engineered intron within the GFP ORF. This construct is based on the pEGFP-N3 construct (Clontech). An empty construct without a microRNA precursor was used as a control.

To stably overexpress miR-126 in mouse ES cells, a lentiviral vector expressing mouse pri-miR-126 (−500 bp total) under control of the EF1-αpromoter, containing a blasticiding resistance cassette, was constructed. Ivey et al. (2008) Cell Stem Cell 2:219. ES cells were stably transfected with the miR-126 expression construct or an empty construct and pools of blasticidin-resistant clones were selected.

### Transfection/Electroporation of Plasmids, MOs and MicroRNA Mimics

HeLa cells were transfected using Lipofectamine 2000 according to the manufacturer's recommendations. Cells were transfected at 90% confluency in six-well dishes with 2 μg of pGL3 (empty or with 3′ UTR of potential targets inserted), 2 μg of expression construct (empty or with miR-1 or miR-126 pri-cursor sequence), and 0.1 μg of Renilla construct (for normalizing transfection efficiency). Cells were analyzed at 48 h post-transfection.

Human umbilical vein endothelial cells (HUVECs) (0.5×10⁶) were electroporated using the Amaxa Nucleofector according to the manufacturer's recommendations, with 15 nmol of control MO or MOs that block processing of the miR-126 pri-cursor or translation of SPRED1 (Gene Tools; see below for sequences). For rescue experiments, HUVECs were co-electroporated with 15 nmol of control or miR-126 MOs together with SPRED1 MO. For luciferase experiments, HUVECs were transfected with 1 μg of pGL3 luciferase constructs and 0.5 μg Renilla construct, together with MOs. Cells were analyzed 72 h post-transfection. For transfection of endothelial cells with miR-126 mimic, Oligofectamine (Invitrogen) was used according to the manufacturers's recommendations with 300 nM of control or miR-126 mimic (Dharmacon). Cells were analyzed 48 h post-transfection.

For siRNA-mediated knockdown of PIK3R2, endothelial cells were transfected with 300 nM Stealth siRNA (top strand: 5′-UUG UCG AUC UCU CUG UUG UCC GAG G-3′; SEQ ID NO:47) or a GC-matched control (Invitrogen) using Oligofectamine. For rescue experiments, HUVECs were electroporated with control or miR-126 MO using the Amaxa kit, and then following 24 h were transfected with control or PIK3R2 siRNAs. Analysis of protein or RNA was performed after an additional 48 h.

### Luciferase Assays

Firefly and Renilla luciferase activities were quantified in lysates using the Dual Luciferase Reporter Assay kit (Promega) on a Victor³ 1420 multilabel counter (PerkinElmer), according to the manufacturer's recommendations. Luciferase readings were corrected for background, and Firefly luciferase values were normalized to Renilla to control for transfection efficiency.

### Tube Formation Assays

The ability of HUVECs to form capillary-like tubes in culture was assessed by adding 8×10⁴ cells to 250 μL of pre-gelled Matrigel (BD Biosciences) in 1 mL of complete medium (ScienCell). The extent of tube formation was assessed at various time-points following seeding.

### Migration/Scratch Assays

Migration of endothelial cells was monitored by generating a ‘scratch’ in a monolayer of confluent endothelial cells with a P1000 pipet tip and observing the extent of wound closure after 8 or 24 h. These experiments were performed with complete medium or in basal medium (no FBS or growth factors) with 50 ng/mL of vascular endothelial growth factor (VEGF).

### MicroRNA Arrays

RNA was extracted from CD31-positive and -negative cells isolated by FACS from day 7 (d7) or day 14 (d14) EBs. 1 μg of RNA was used for microRNA array analysis using Exiqon arrays. Endothelial-enriched microRNAs were identified based on a 1.5-fold enrichment of expression in CD31-positive versus CD31-negative cells.

### Expression Arrays

HUVECs were transfected with 15 nmol of standard control or miR-126 antisense MOs (Gene-Tools) with the Amaxa Nucleofector. After 72 h, RNA was extracted, labeled with biotin, and used for hybridization to Affymetrix Human Gene ST 1.0 arrays. Arrays were performed on three biological replicates. Raw intensities from the CEL files were analyzed using Affymetrix Power Tool (APT, version 1.8.5) to generate robust multi-array average intensity in log2 scale for each probe set. The perfect match intensities for each probe set were corrected for background and quantile-normalized using a robust fit of linear models. Differential expression was determined using the limma package in R/Bioconductor.

For zebrafish arrays, approximately 5 ng of RNA from sorted Tg(flk1:GFP)^{s843}-expressing cells from 48 hours post-fertilization (hpf) embryos were amplified using the NuGen WT-Ovation Pico amplification kit, and 5 μg of amplified cDNA was biotin-labeled using the WT-Ovation cDNA Biotin Module V2 and hybridized to Affymetrix Zebrafish Genome arrays. Arrays were performed using four biological replicates of control zebrafish and zebrafish injected with two independent miR-126 MOs. Human orthologs of zebrafish genes were extracted from HomoloGene (build 59, dated February 2008) and mapped to probe sets with EntrezGene IDs. This yielded 5543 orthogolous pairs of zebrafish-human gene pairs. Gene Ontology analysis was performed using GOStat (see the following internet site: http://gostat(dot)wehi(dot)edu(dot)au).

### Quantification of Gene Expression Using Quantitative Reverse Transcription PCR

To analyze microRNA expression by qRT-PCR, 10 ng of RNA was reverse transcribed using microRNA-specific primers from Applied Biosystems or Qiagen. Real-time PCR was performed on diluted samples with miR-16 as an internal control. To compare miR-126 to miR-126* expression, standard curves were generated using a known amount of miR-126 or miR-126* mimic (Dharmacon). For quantification of mRNA expression, first-strand synthesis was performed on 1 μg of RNA using SuperScript III (Invitrogen). After diluting to a final volume of 100 μL, 2 μL was used in triplicate for real-time PCR with an ABI 2100 real-time PCR thermocyler. Taqman gene expression assays were purchased from Applied Biosystems. Alternatively, primer sets were designed using Vector NTI, and Sybr green technology (Applied Biosystems) was used to quantify gene expression. All primer sets for mRNAs crossed an exon-exon junction to avoid the amplification of genomic DNA. The expression of TATA box binding protein (TBP) and GAPDH (glyceraldehyde 3-phosphate dehydrogenase) were used as controls for mRNA expression. Gene expression changes were quantified using the delta-delta C_{T} method. Primer sequences are available upon request.

### Chromatin Immunoprecipitation (ChIP)

ChIP was performed as described (Fish et al. (2005) J. Biol. Chem. 280:24824), with antibodies directed to the large subunit of RNA Pol II (N-20, Santa Cruz). ChIP was performed on approximately 10⁶ endothelial cells that were transfected with control or miR-126 MOs for 72 h Immunoprecipitations were done with 2 μg of antibody, and a control with no antibody was performed in parallel. Samples were resuspended in 30 μL of water. The density of Pol II was determined by quantifying the number of copies of the target amplicon (EGFL7 promoter or coding region) in the Pol II sample, subtracting the number of copies in the no antibody control and dividing by a diluted input sample that was removed before immunoprecipitation. Human genomic DNA was used to generate a standard curve for quantification.

### Zebrafish Experiments

Tg(flk1: GFP)^{s843}; Tg(gata1:dsRed)^{sd2} zebrafish embryos (Jin et al. (2007) Dev. Biol. 307:29) were injected at the one-cell stage with 4 ng of MO. For spred1 mRNA injection, full-length zebrafish spred1 was cloned into pcDNA3.1. mRNA was generated by T7 mMessage mMachine (Ambion), and 100 pg of mRNA was injected into embryos. As a control, zebrafish heart adapter protein 1 (hadp1) mRNA (Accession number: EU380770) was synthesized and injected in parallel. While development was essentially normal with 100 pg of injected hadp1, developmental abnormalities were evident at higher amounts of injected mRNA. Embryo development was assessed at 24-72 hpf.

### Morpholinos

MOs targeting dre-pri-miR-126 were 5′-TGC ATT ATT ACT CAC GGT ACG AGT TTG AGT C-3′ (MO-1; SEQ ID NO:19) and GCC TAG CGC GTA CCA AAA GTA ATA A (MO-2; SEQ ID NO:20), and those targeting hsa-miR-126 were 5′-GCA TTA TTA CTC ACG GTA CGA GTT T (SEQ ID NO:21). To direct nonsense-mediated decay of zebrafish spred1, a MO was designed to cause exon 2 skipping and the generation of a premature stop codon. The MO was 5′-CCT GAG GAC CAG AAA CAG TCT CAC C (SEQ ID NO:22). To block translation of human SPRED1, the following MO was used: 5′-GTC TCC TCG CTC ATC TTT CCC TCA C (SEQ ID NO:23).

### Western Blotting

Western blots were performed with 20-40 μg of protein. The antibodies used were anti-AKT1 (Santa Cruz), phospho-AKT1/2/3 (Cell Signaling), ERK2 (Santa Cruz), -phospho-ERK1/2 (Cell Signaling), -EGFL7 (Santa Cruz or Abnova), -SPRED1 (Abgent and kindly provided by Dr. Yoshimura), -PIK3R2 (Abcam), -GAPDH (Santa Cruz), and VCAM1 (Santa Cruz). For phospho-AKT and -ERK western blots, HUVECs were serum-starved in medium containing 0.1% FBS without growth factors overnight and then stimulated with 10 ng/mL of human recombinant vascular endothelial growth factor (VEGF) (BD Biosciences) for 10 min.

### Statistical Analyses

All experiments were repeated a minimum of three times, and the error bars on graphs represent the mean±the standard error of the mean, unless otherwise stated. Statistical significance was determined by a Student's T-test or ANOVA, as appropriate and a p-value of <0.05 was considered as significant.

### Results

miR-126 is the Most Highly Enriched MicroRNA In Endothelial Cells

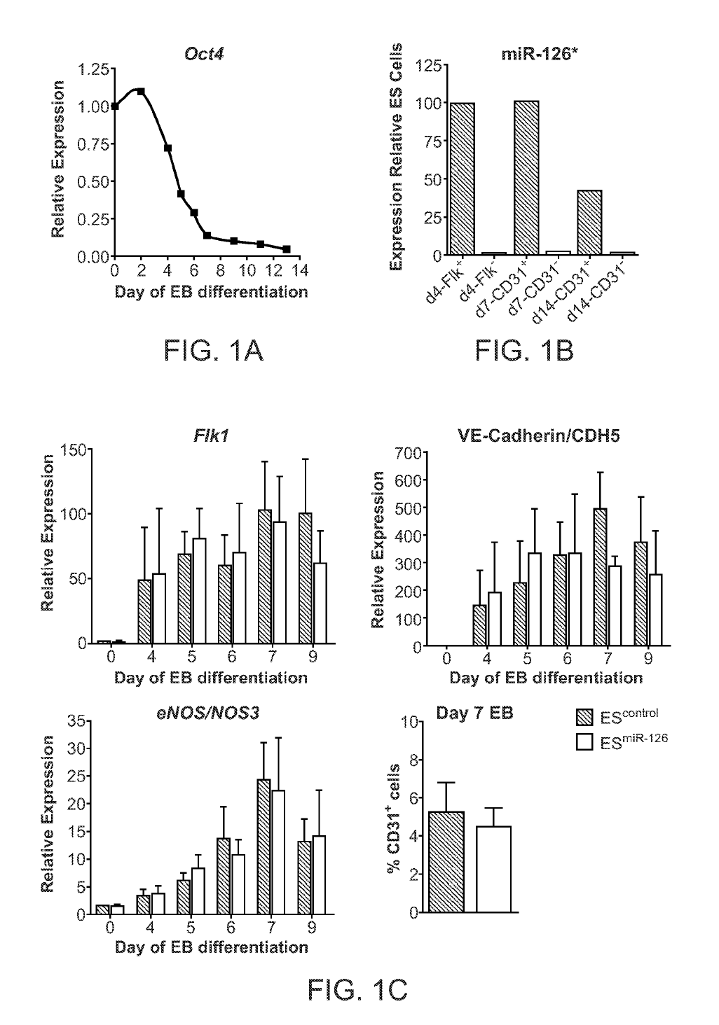

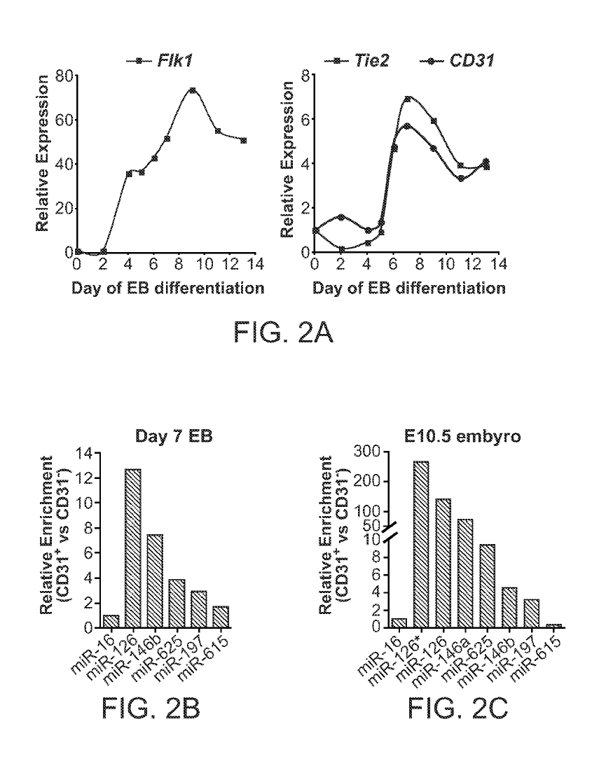

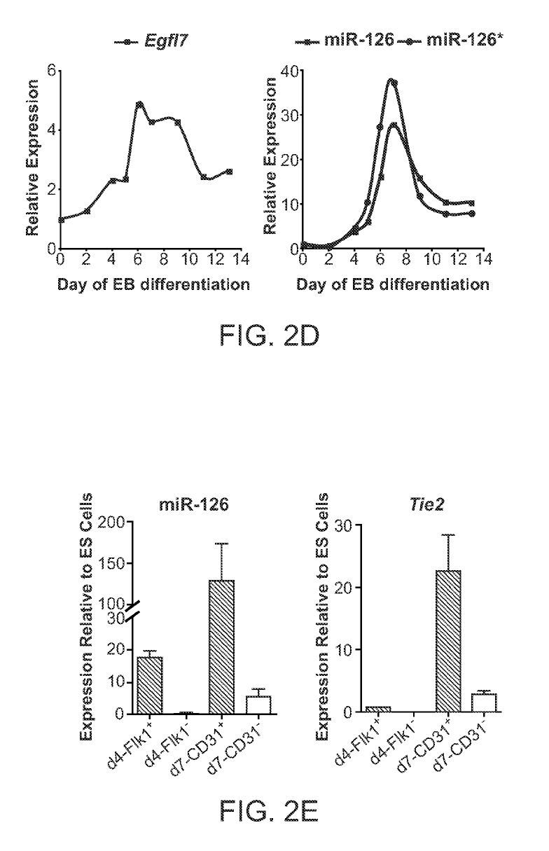

To determine when the endothelial lineage first appears in differentiating mouse embryonic stem (ES) cells in the embryoid body (EB) model, extensive mRNA expression profiling was performed for endothelial marker expression using quantitative reverse transcriptase PCR (qRT-PCR). Oct4, a marker of pluripotent ES cells, was rapidly down-regulated during differentiation (FIG. 1A). At day 4 (d4) of EB formation, Flk1/Vegfr2 was dramatically induced, but other endothelial markers, such as Tie2/Tek and CD31/Pecam1, were unchanged (FIG. 2A). This population of Flk1-positive cells at day 4 (d4) contains vascular precursor cells, as evidenced by the ability of isolated Flk1-positive cells to differentiate into the endothelial lineage in the presence of VEGF. By day 6 (d6) of EB formation, endothelial markers, including CD31 and Tie2, were robustly expressed and remained elevated even after 14 days of differentiation.

CD31-positive and -negative cells were isolated from day 7 (d7) EBs by fluorescence-activated cell sorting (FACS) and performed microRNA microarray profiling. While several microRNAs, including miR-146b, miR-197, miR-615, and miR-625, were enriched more than 1.5-fold in CD31-positive cells, miR-126 was the most highly enriched microRNA (FIG. 2B). microRNA arrays were also performed at d14 of EB formation, and the same subset of microRNAs was enriched, suggesting that relatively few microRNAs are enriched in endothelial cells.

To determine if these microRNAs were also enriched in endothelial cells in vivo, FACS was used to isolate CD31-positive cells from E10.5 mouse embryos. Quantitative reverse transcription-polymerase chain reaction (qRT-PCR) with RNA from these cells confirmed the enrichment of the above microRNAs, with the exception of miR-615, in CD31-positive cells compared to CD31-negative cells from the same embryos (FIG. 2C). Additionally, miR-126*, expressed from the opposite strand of the miR-126 pre-miRNA, was also highly enriched in endothelial cells in vivo, as was miR-146a, which differs from miR-146b by only two nucleotides near the 3′ end of the mature microRNA (FIG. 2C).

miR-126 is located in an intron of Egfl7, a gene that is highly expressed in endothelial cells (Parker et al. (2004) Nature 428:754). The expression of Egfl7 largely mirrored that of endothelial markers during EB formation (FIG. 2D). Interestingly, Egfl7, miR-126 and miR-126* were induced at d4 of EB formation and further induced at d6, when endothelial markers were robustly expressed. This finding suggested that miR-126 may be expressed in vascular progenitors. FACS was used to isolate Flk1-positive cells from EBs at d4 of differentiation. miR-126 and miR-126* were highly enriched in these vascular progenitors at d4 and were also enriched in mature CD31-expressing endothelial cells (FIG. 2E).

FIGS. 1A-C. miR-126 is not sufficient for the differentiation of pluripotent cells to the endothelial cell lineage. (A) Expression of Oct4, a pluripotency marker was monitored during EB formation by qRT-PCR. (B) miR-126* was enriched in Flk1⁺ vascular precursors (day 4; d4) and in mature, CD31⁺ endothelial cells (day 7 and day 14; d7 and d14). (C) Expression of the endothelial markers, Flk1, VE-Cadherin/CDH5 and eNOS/NOS3 were not altered in EBs derived from miR-126 over-expressing ES cells (ES^{miR-126}). The number of CD31-positive cells measured by FACS at day 7 (d7) of EB formation was also not altered.

FIGS. 2A-E. Identification of microRNAs enriched in endothelial cells. (A) Gene expression changes were monitored by qRT-PCR during differentiation of mouse ES cells in an embryoid body (EB) model. Flk1 is expressed in vascular progenitors and mature endothelial cells, while CD31 and Tie2 are markers of mature endothelial cells. Expression was normalized to Tata-binding protein (Tbp) levels. The average of multiple experiments is shown. (B) Endothelial cells were isolated from day 7 (d7) EBs by cell sorting with anti-CD31 antibodies and microRNA arrays were performed. microRNAs enriched more than 1.5-fold relative to miR-16 in CD31⁺ cells compared to CD31⁻ cells are shown. (C) Enrichment of microRNAs identified in (B) in CD31⁺ endothelial cells sorted from E10.5 mouse embryos assayed by qRT-PCR. (D) Expression of Egfl7, miR-126 and miR-126* in EBs assayed by qRT-PCR. (E) miR-126 was enriched in sorted vascular progenitors (Flk1⁺) from d4 EBs and in endothelial cells (CD31⁺) from d7 EBs compared to ES cells. Tie2 expression was used to assess endothelial gene expression in sorted cells used in (B) and (E).

miR-126 Does not Control Endothelial Lineage Commitment

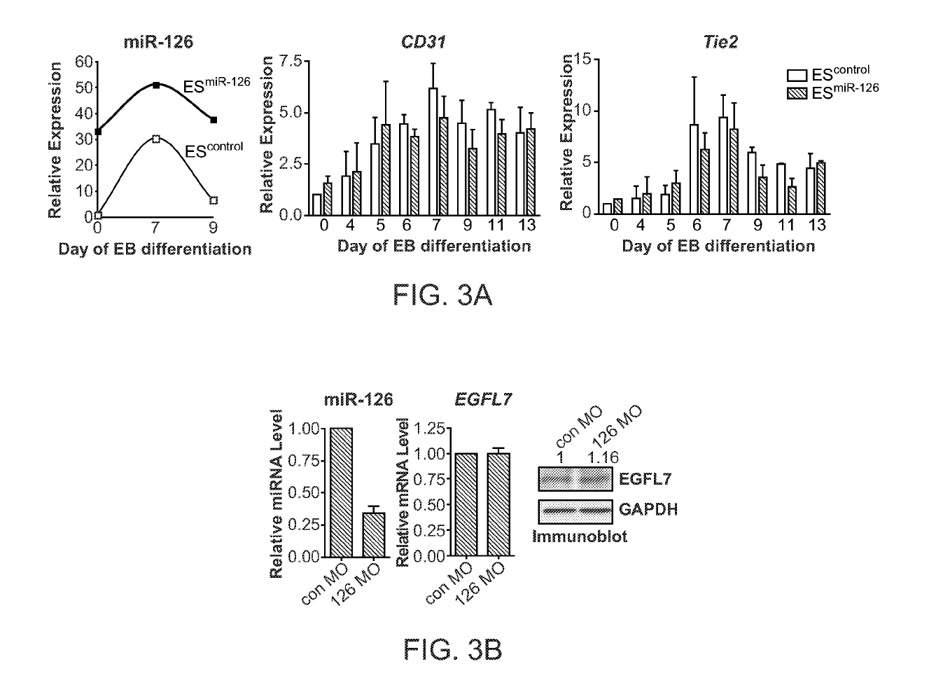

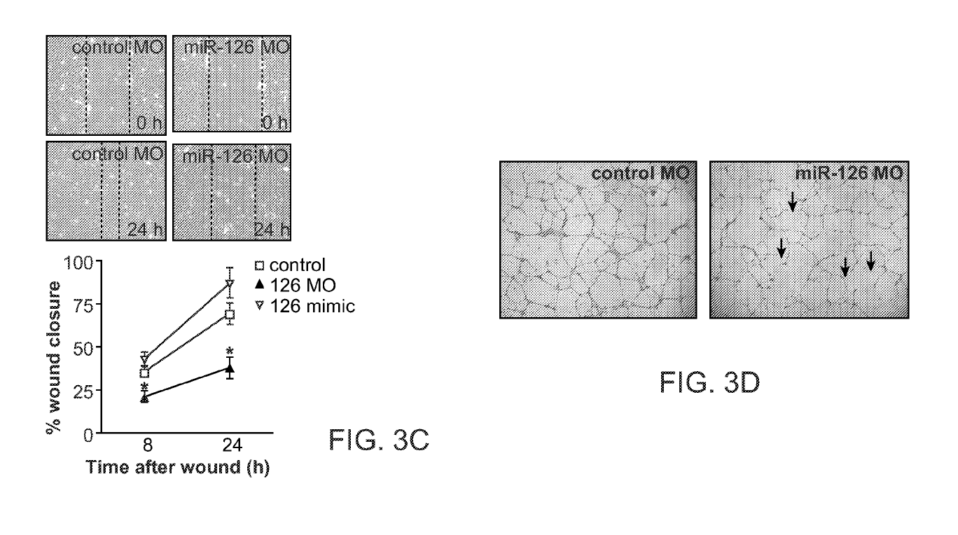

Because of the early induction of miR-126 in vascular progenitors, experiments were conducted to determine if this microRNA might regulate differentiation towards the endothelial lineage. Stable mouse ES cell lines that express miR-126 under control of the ubiquitously-expressed EF1-αpromoter (mES^{miR-126}) were created. Increased expression of miR-126 was documented in mES^{miR-126} cells and in EBs derived from them (FIG. 3A). Analysis of the expression pattern of several endothelial genes, including Flk1, VE-cadherin/CDH5, eNOS/NOS3, Tie2 and CD31, in mES^{control} and mES^{miR-126} cells during differentiation did not reveal any major alterations in endothelial gene expression (FIG. 3A and FIG. 1C), and the number of CD31-positive cells at d7 was not altered (FIG. 1C). This suggests that while miR-126 is enriched in vascular progenitors, it is not sufficient to promote differentiation of pluripotent cells towards the endothelial lineage.

miR-126 modulates the response of endothelial cells to VEGF

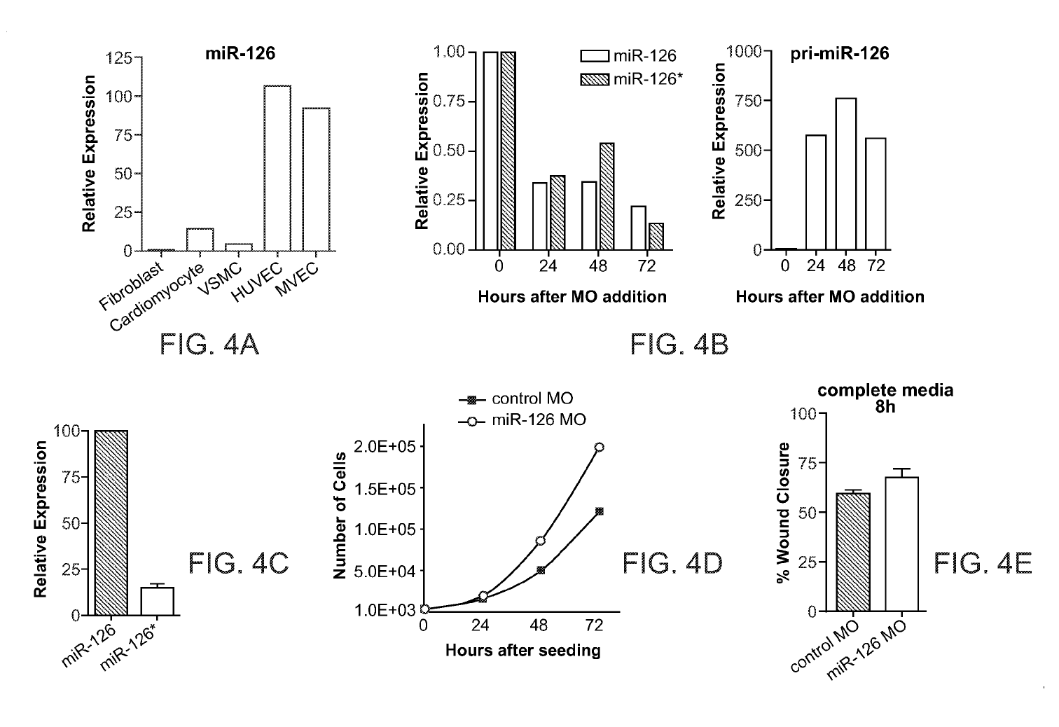

To study the loss-of-function of miR-126 in endothelial cells, a morpholino (MO) antisense to miR-126 that spanned the miR-126 5′ Dicer cleavage site of the miR-126 pri-cursor, was introduced into human umbilical vein endothelial cells (HUVECs). These cells express high levels of miR-126 (FIG. 4A). Introduction of this MO resulted in decreased levels of both mature miR-126 and miR-126* and an increase in miR-126 pri-cursor, beginning at 24 hour (h) post-transfection (FIG. 4B). While both miR-126 and miR-126* were reduced to a similar extent, the absolute basal level of miR-126 was much higher than miR-126* in endothelial cells (FIG. 4C). Importantly, levels of spliced EGFL7 mRNA detected by qRT-PCR with primers surrounding the intron containing miR-126, and protein levels of EGFL7, were unaffected by introduction of this MO (FIG. 3B).

Endothelial cells with reduced levels of miR-126 were phenotypically indistinguishable from control MO-transfected cells, but had elevated proliferation rates (FIG. 4D). The endothelial phenotype was further studied in an in vitro wound closure assay, in which the rate of migration of cells into a denuded area of a confluent monolayer was monitored. Modulating miR-126 levels had no effect on cell migration when complete medium was used (FIG. 4E). However, VEGF-induced migration was inhibited in miR-126 knockdown cells compared to control MO-transfected cells (FIG. 3C). Conversely, in cells transfected with miR-126 mimic, which resulted in a 50-fold increase in levels of miR-126, there was a trend towards increased migration in response to VEGF stimulation (FIG. 3C). These data suggest that VEGF-dependent endothelial cell migration is regulated by miR-126 abundance. The effects of miR-126 on the formation and stability of capillary tubes on matrigel were also assessed. While initial formation of tubes appeared normal, the capillary tubes were less stable and appeared thin, with dissociation of many tubes within 24 h (FIG. 3D). This suggests that miR-126 may play a role in regulating vessel stability.

FIGS. 3A-D. miR-126 regulates endothelial migration and capillary tube stability in vitro. (A) miR-126 levels were measured in ES^{control} and ES^{miR-126} cells at various stages of EB differentiation by qPCR (left panel). qRT-PCR of CD31 and Tie2, two endothelial-restricted transcripts, in ES^{control} and ES^{miR-126} cells at progressive days of EB differentiation shows no difference in endothelial cell lineage determination from pluripotent cells (right panels). (B) Relative levels of mature miR-126, spliced EGFL7 mRNA across the miR-126-containing intron, and EGFL7 protein (immunoblot) were measured in HUVECs transfected with miR-126 morpholinos (MOs) or control (con) MO. Densitometric analysis of EGFL7 protein levels are indicated above. (C) The effect of miR-126 knockdown (MO) and over-expression (mimic) on endothelial cell migration was determined by generating a “scratch” in a confluent monolayer of endothelial cells and measuring the degree of “wound closure” after 8 and 24 h. Dashed lines indicate width of “wound”. Percent wound closure is shown as the mean±SEM of five scratches from one representative experiment. *p<0.05 compared to control. (D) Capillary tube formation of endothelial cells transfected with control or miR-126 MOs and seeded onto Matrigel. Capillary tubes formed in endothelial cells transfected with miR-126 MOs were unstable, exhibiting frequent dissociation of tubular structures (arrows).

FIGS. 4A-E. Phenotypic analysis of endothelial cells with altered miR-126 expression. (A) miR-126 expression was quantified by qRT-PCR in several primary human cell types; cardiac fibroblasts, cardiac myocytes, vascular smooth muscle (VSMC), human umbilical vein endothelial cells (HUVEC) and dermal microvascular endothelial cells (MVEC). (B) Introduction of a miR-126 MO into HUVECs resulted in decreased levels of mature miR-126 and miR-126* (left) and an increase in the levels of the pri-cursor for miR-126 (right). (C) miR-126 is more abundant in human endothelial cells than miR-126* as determined by qRT-PCR. Standard curves were generated with known amounts of miR-126 and miR-126* mimics to determine absolute copy numbers. (D) Cells with reduced miR-126 levels proliferated at a more rapid rate than control cells. (E) Migration of endothelial cells in a scratch assay in complete media was not significantly affected by miR-126 knockdown.

miR-126 Regulates Blood Vessel Stability In Vivo

Considering the dramatic effect of miR-126 on the behavior of human endothelial cells in vitro, the in vivo function of miR-126 was assessed. For this purpose zebrafish was used as a model system, in which a functioning cardiovascular system is not required for viability through relatively advanced stages of embryogenesis. The mature forms of zebrafish miR-126 and miR-126* are identical to their human orthologues.

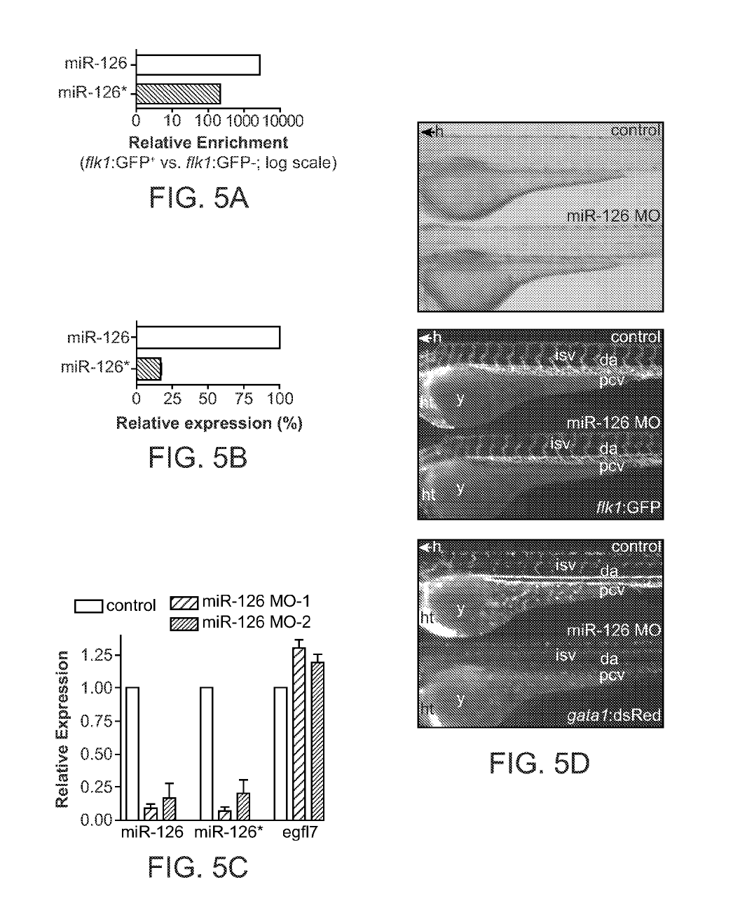

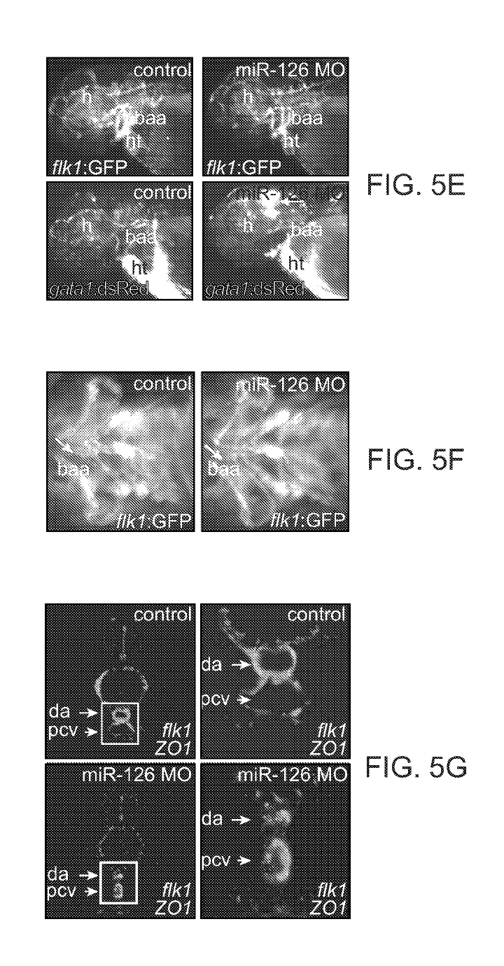

FACS isolation of GFP-positive cells from the endothelial cell-specific zebrafish reporter line, Tg(flk1:GFP)^{s843} (Jin et al. (2007) supra), demonstrated that miR-126 and miR-126* were highly enriched in zebrafish endothelial cells (FIG. 5A). As in human endothelial cells, miR-126 was more abundant than miR-126* in zebrafish embryos (FIG. 5B). miR-126 expression was decreased during zebrafish development by injecting two unique morpholinos (miR-126 MO1 and MO2 (FIG. 6A)) into fertilized eggs. Injection of these MOs blocked processing of pri-miR-126, resulting in a profound decrease in mature levels of miR-126 and miR-126* (FIG. 5C). Importantly, levels of egfl7, which hosts one of the two copies of zebrafish miR-126 and regulates tubulogenesis in zebrafish (Parker et al. (2004) Nature 428:754), were not dramatically altered by the miR-126 MOs (FIG. 5C).

Through the use of Tg(flk1:GFP)^{s843}; Tg(gata1:dsRed)^{sd2} zebrafish, which express GFP in the vasculature, and dsRed in blood cells, the effect of miR-126 knockdown on vascular development and circulation was assessed. Between 48-72 hpf hours post-fertilization (hpf), no differences in gross morphology (FIG. 5D, top panel) or vascular patterning (FIG. 5D, middle panel) were evident between control and miR-126 morpholino-injected (morphant) embryos. Additionally, FACS quantification revealed no significant difference in the percentage of Tg(flk1:GFP)^{s843}-expressing endothelial cells in control, miR-126 MO1 or miR-126 MO2-treated zebrafish (72 hpf) (2.38±0.38%, 2.70±0.40%, 3.06±0.62%, respectively). However, several functional abnormalities were evident in the circulation and vessel morphology. These defects occurred in >70% of the miR-126 MO-injected embryos, and were similar with either MO. The presence of Tg(gata1:dsRed)^{sd2}-expressing blood cells in the head vasculature, intersomitic vessels (ISVs), dorsal aorta (DA), and primary cardial vein (PCV) was reduced between 48 and 72 hpf (FIG. 5D (bottom panel), FIG. 5E). However, blood cells were made and were visible in the heart (ht) (FIGS. 5D, E). Severe hemorrhages were also evident in the head, evidenced by the accumulation of dsRed-positive cells (FIG. 5E). Importantly, heart function and morphology were not obviously affected by miR-126 MO at either 48 or 72 h.

It was observed that some Tg(gata1:dsRed)^{sd2}-expressing cells were trapped in the ISVs of miR-126 morphants, indicating that blood vessel integrity might be compromised. Indeed, branchial arch vessels appeared to have a reduced lumen diameter in morphants (FIG. 5F). To better characterize these defects endothelial tube integrity of the DA and PCV was analyzed by confocal analyses of embryo cross-sections (FIG. 5G). These experiments revealed collapsed lumens and compromised endothelial integrity in miR-126 morphants, suggesting that miR-126 expression is required to maintain vessel integrity and caliber during zebrafish vascular development. This phenotype correlates well with our in vitro findings of a decrease in the stability of capillary networks in miR-126 knockdown human endothelial cells.

FIGS. 5A-G. miR-126 regulates vascular integrity and lumen maintenance in vivo. (A) miR-126 and miR-126* enrichment (qRT-PCR) in GFP endothelial cells from 72 hpf Tg(flk1:GFP)^{s843} zebrafish compared to GFP⁻ cells. (B) Relative levels of miR-126 and miR-126* compared to known standards by qRT-PCR in 72 hpf zebrafish embryos. (C) Levels of miR-126/126* or egfl7 quantified by qRT-PCR in 72 hpf zebrafish injected with miR-126 MOs relative to control. Expression of the egfl7 transcript, measured across intron containing miR-126, was not markedly affected by MO injection. (D) Lateral views of control and miR-126 MO-injected Tg(flk1:GFP)^{s843}; Tg(gata1:dsRed)^{sd2} zebrafish (72 hpf). Brightfield (top) reveals no major changes in morphology, while flk1:GFP shows no alteration in blood vessel patterning (middle). Presence of blood cells (gata1:dsRed) in the intersomitic vessels (isv), dorsal aorta (da) and primary cardinal vein (pcv) was greatly reduced in morphants (bottom). y=yolksac, h=head, ht=heart. (E) miR-126 morphants had normal vessel patterning (flk1:GFP), but developed cranial hemorrhages (gata1:dsRed; arrow) in the head (h). ht=heart, baa=branchial arch arteries. (F) Ventral view of BAA suggested smaller luminal size (indicated by arrow) in miR-126 morphants. (G) Transverse section of control or miR-126 MO-treated fish revealed that the DA and PCV of morphants had a smaller lumen size than controls; higher magnification (right panels) of boxed area shows collapsed DA and small PCV in morphants. ZO-1 is an epithelial marker.

FIGS. 6A and 6B. (A) Schematic of antisense MOs used to block miR-126/126* expression in zebrafish. miR-126 and miR-126* are indicated in red. (B) Schematic of miR-126 binding sites in predicted human miR-126 target mRNAs. Complementary nucleotides indicated by vertical bars and G:U wobble indicated by “:”.

Identification of Genes Regulated by miR-126 by Microarray

Although miR-126 morphants had severe defects in vessel stability, the number of endothelial cells was not altered. Tg(flk1:GFP)^{s843}-expressing endothelial cells were isolated by FACS from control and miR-126 MO1- and MO2-injected fish and analyzed mRNA expression by microarray. Since similar genes were altered in miR-126 MO1 and MO2 injected fish, the data sets were combined to identify dysregulated genes in miR-126 morphants (see Tables 1 and 2 for up- and down-regulated genes, respectively).

Table 1 depicts select genes upregulated (>1.5 fold) in endothelial cells isolated from zebrafish injected with miR-126 morpholino (p>0.05). Table 2 depicts genes downregulated (<−1.5-fold) in endothelial cells isolated from zebrafish injected with miR-126 morpholino (p>0.05).

TABLE 1

| Entrez Gene | Gene Symbol | Gene Name | Fold Change | P-value |
|---|---|---|---|---|
| 565439 | slc12a3 | solute carrier family 12, member 3 | 1.93 | 4.60E−05 |
| 550569 | mylc2pl | myosin light chain 2, precursor lympocyte- | 1.84 | 0.033 |
| | | specific | | |
| 337731 | pvalb4 | parvalbumin 4 | 1.79 | 0.037 |
| 321552 | smyhc1 | slow myosin heavy chain 1 | 1.79 | 0.018 |
| 415223 | murf1 | muscle specific ring finger protein 1 | 1.78 | 0.014 |
| 408256 | actc1l | actin, alpha, cardiac muscle 1 like | 1.77 | 0.009 |
| 558036 | lmx1a | LIM homeobox transcription factor 1 alpha | 1.77 | 0.0098 |
| 569876 | rgs4 | regulator of G-protein signaling 4 | 1.77 | 0.00017 |
| 30148 | desmin | desmin | 1.72 | 0.023 |
| 406588 | sstr5 | somatostatin receptor 5 | 1.72 | 0.00041 |
| 337514 | mpx | myeloid-specific peroxidase | 1.71 | 3.30E−05 |
| 58074 | dct | dopachrome tautomerase | 1.69 | 0.014 |
| 573301 | nexn | nexilin (F-actin binding protein) | 1.68 | 0.0063 |
| 360136 | penk | proenkephalin | 1.68 | 0.024 |
| 30084 | ndpkz2 | nucleoside diphosphate kinase-Z2 | 1.68 | 0.023 |
| 406496 | aldoab | aldolase a, fructose-bisphosphate b | 1.67 | 0.013 |
| 30344 | hoxb9a | homeo box B9a | 1.65 | 0.00032 |
| 449648 | hoxc8a | homeo box C8a | 1.60 | 0.0026 |
| 321035 | pabpc4 | poly(A) binding protein, cytoplasmic 4 | 1.60 | 0.00065 |
| 554697 | slc16a3 | solute carrier family 16, member 3 | 1.60 | 3.10E−05 |
| 393999 | pgam2 | phosphpoglycerate mutase 2 | 1.57 | 0.029 |
| 30234 | tnw | tenascin W | 1.56 | 0.00024 |
| 393874 | tmem38a | transmembrance protein 38A | 1.56 | 0.0016 |
| 323320 | actn3 | alpha actinin 3 | 1.56 | 0.011 |
| 30591 | hsp90a | heat shock protein 90-alpha 1 | 1.55 | 0.0011 |
| 140634 | cyp1a | cytochrome P450, family 1, subfamily A | 1.55 | 0.0082 |
| 436878 | sirt5 | sirtuin 5 | 1.55 | 0.00013 |

TABLE 2

| Entrez Gene | Gene Symbol | Gene Name | Fold Change | P-value |
|---|---|---|---|---|
| 30262 | Ins | Preproinsulin | −2.91 | 5.80E−05 |
| 378986 | fga | fibrinogen alpha chain | −2.77 | 0.00017 |
| 30255 | tfa | transferrin-a | −2.41 | 0.00026 |
| 326018 | sst1 | somatostatin 1 | −2.00 | 7.10E−05 |
| 492490 | slc6a1 | solute carrier family 6, member 1 | −1.99 | 0.0015 |
| 79185 | gcga | glucagon a | −1.82 | 0.003 |
| 100000329 | hbae1 | hemoglobin alpha embryonic 1 | −1.80 | 0.0093 |
| 114415 | atoh2b | atonal homolog 2b | −1.75 | 0.049 |
| 30601 | hbae3 | hemoglobin alpha embryonic 3 | −1.72 | 0.012 |
| 797346 | spint1 | serine peptidase inhibitor, Kunitz type 1 | −1.69 | 0.0062 |
| 337132 | anxa5 | annexin A5 | −1.68 | 0.00012 |
| 405890 | esrrg | estrogen-related receptor gamma | −1.65 | 0.002 |
| 100000558 | vip | vasoactive intestinal polypeptide | −1.64 | 0.0022 |
| 100004501 | fgg | fibrinogen, gamma polypeptide | −1.64 | 0.0059 |
| 337315 | fgb | fibrinogen, B beta polypeptide | −1.63 | 0.0011 |
| 406303 | tuba2 | tubulin, alpha 2 | −1.59 | 0.013 |
| 445095 | tm4sf4 | transmembrane 4 suberfamily, member 4 | −1.59 | 0.009 |
| 556665 | nfia | nuclear Factor I/A | −1.59 | 0.00045 |
| 563087 | sema4d | semaphorin 4D | −1.59 | 0.00032 |
| 30038 | sox19a | SRY-box containing gene 19a | −1.58 | 2.30E−05 |
| 405772 | hbbe2 | hemoglobin beta embryonic 2 | −1.57 | 0.02 |
| 100004700 | cyp24a1l | cyp24a1 like | −1.54 | 0.016 |
| 565575 | smg7 | smg7-homolog | −1.53 | 0.0078 |
| 560026 | dsg2 | desmoglein 2 | −1.53 | 0.0031 |
| 30542 | foxb1.2 | forkhead box B1.2 | −1.52 | 0.012 |
| 81586 | cldng | claudin g | −1.52 | 0.0059 |

By Gene Ontology (GO) statistical analysis the most highly dysregulated class of genes in the endothelium of miR-126 morphants encoded transcription factors (Table 3). The homeobox (HOX) and forkhead box (FOX) family of genes were especially affected in miR-126 morphants, many of which regulate endothelial cell biology, including angiogenesis (Bruhl et al. (2004) Circ. Res. 94:743; Dejana et al. (2007) Biochmi. Biophys. Acta 1775:298; Myers et al. (2000) J. Cell Biol. 148:343. Table 3 depicts GO terms over-represented among genes altered by <−1.3 fold or >1.3 fold in zebrafish endothelial cells isolated from embryos injected with miR-126 morpholino (p<0.01). Upregulated genes are indicated in bold.

TABLE 3

| GO Term | Genes |
|---|---|
| Sequence-specific | cdx4, cebpd, crem, esr1, esrrg, fosl2, foxd5, |
| DNA binding | foxg1, foxi1, gbx2, hoxa2b, hoxa9b, |
| GO: 0043565, | hoxa10b, hoxb1a, hoxb1b, hoxb7a, hoxb8a, |
| p = 2.58E−08 | hoxb9a, hoxb10a, hoxc8a, hoxc11a, |
| | hoxd9a, hoxd11a, hoxd12a, krml2, krml2.2, |
| | lmx1a, pou12, rad51, thrb |
| Regulation of | atoh8, bhlhb2, cdx4, cebpd, crem, egr1, esr1, |
| transcription | esrrg, fosl2, foxd5, foxg1, foxi1, gbx2, |
| GO: 0045449, | hif1al2, hoxa2b, hoxa9b, hoxa10b, hoxb1a, |
| p = 9.89E−05 | hoxb1b, hoxb7a, hoxb8a, hoxb9a, hoxb10a, |
| | hoxc8a, hoxc11a, hoxd9a, hoxd11a, |
| | hoxd12a, krml2, krml2.2, lmx1a, myf5, |
| | myog, pou12, sirt5, sox19a, tbx15, tcea3, |
| | thrb |

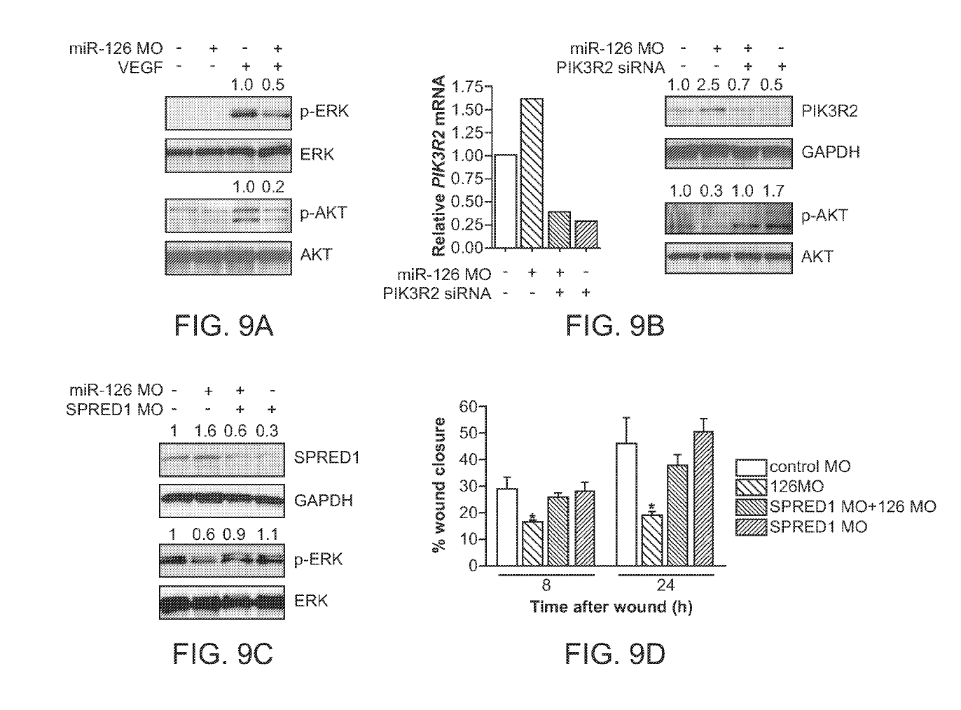

Microarray analysis was also performed with RNA from human endothelial cells (HUVECs) in which miR-126 was knocked-down for 72 h (see Tables 4 and 5, for up- and down-regulated genes, respectively). The most over-represented GO terms were related to the cell cycle and the cytoskeleton (Table 6). This observation supports the finding that cells with reduced levels of miR-126 proliferated more rapidly than control cells (FIG. 9D). Platelet-derived growth factors (PDGF) A, B, C and D, which are important in endothelial biology, were all significantly down-regulated in cells with reduced levels of miR-126 (Table 6). In addition, genes categorized as important for vascular development were highly dysregulated (Table 6). A total of 61 genes were similarly altered (p<0.05) in zebrafish and human miR-126 knockdown expression arrays, suggesting a high conservation in the gene repertoire regulated by miR-126. While many of the dysregulated genes were likely not direct targets of miR-126, the findings of this array suggest that miR-126 regulates several aspects of endothelial biology, including proliferation, cytoskeletal function, PDGF signaling, and vascular development.

Table 4 depicts select genes upregulated (>1.5 fold) in human endothelial cells treated with miR-126 morpholino (p>0.01). Bold indicates a predicted target of miR-126. Table 5 depicts genes downregulated (<−1.5-fold) in endothelial cells isolated from zebrafish injected with miR-126 morpholino (p>0.01) Table 6 depicts GO terms over-represented among genes altered by <−1.5 fold or >1.5 fold in human endothelial cells treated with miR-126 morpholino (p<0.01). Upregulated genes are indicated in bold.

TABLE 4

| Genbank | Gene Symbol | Gene Name | Fold Change | P-value |
|---|---|---|---|---|
| — | hsa-miR-126 | | 62.5 | 2.4E−08 |
| NM_201446 | EGFL7 | EGF-like-domain, multiple 7 | 2.78 | 2.6E−05 |
| NM_005266 | GJA5 | Gap junction protein, alpha 5, 40 kDa | 2.50 | 4.6E−06 |
| NM_003816 | ADAM9 | ADAM metallopeptidase domain 9 (meltrin | 1.94 | 1.4E−05 |
| | | gamma) | | |
| NM_005824 | LRRC17 | Leucine rich repeat containing 17 | 1.83 | 3.4E−05 |
| NM_012319 | SLC39A6 | Solute carrier family 39 (zinc transporter), | 1.82 | 0.00013 |
| | | member 6 | | |
| NM_014730 | KIAA0152 | KIAA0152 | 1.80 | 0.00016 |
| NM_005810 | KLRG1 | Killer cell lectin-like receptor, subfamily | 1.80 | 5.8E−05 |
| | | G, member 1 | | |
| NM_018712 | ELMOD1 | ELMO/CED-12 domain containing 1 | 1.80 | 2.6E−05 |
| NM_030650 | KIAA1715 | KIAA1715 | 1.77 | 0.00034 |
| NM_003535 | HIST1H3J | Histone cluster 1, H3j | 1.72 | 4.5E−05 |
| NM_004701 | CCNB2 | Cyclin B2 | 1.72 | 0.00049 |
| NM_001039724 | NOSTRIN | Nitric oxide synthase traffiker | 1.71 | 9.1E−05 |
| NM_003521 | HIST1H2BM | Histone cluster 1, H2bm | 1.70 | 0.00084 |
| NM_005733 | KIF20A | Kinesin family member 20A | 1.70 | 0.00058 |
| NM_016359 | NUSAP1 | Nucleolar and spindle associated protein 1 | 1.69 | 0.00078 |
| NM_004956 | ETV1 | Ets variant gene 1 | 1.67 | 0.00045 |
| NM_005019 | PDE1A | Phosphodiesterase 1A, calmodulin-dependent | 1.66 | 0.00061 |
| NM_018685 | ANLN | Anillin, actin binding protein | 1.65 | 0.00036 |
| NM_012310 | KIF4A | Kinesin family member 4A | 1.63 | 2.0E−04 |
| NM_145697 | NUF2 | NDC80 kinetochore complex component | 1.59 | 0.00012 |
| NM_016195 | MPHOSPH1 | M-phase phosphoprotein 1 | 1.58 | 0.00061 |
| NM_022909 | CENPH | Centromere protein H | 1.58 | 9.8E−05 |
| NM_005192 | CDKN3 | Cyclin-dependent kinase inhibitor 3 | 1.56 | 0.00011 |
| NM_003617 | RGS5 | Regulator of G-protein signaling 5 | 1.55 | 0.00026 |
| NM_004093 | EFNB2 | Ephrin-B2 | 1.54 | 0.00035 |
| NM_202002 | FOXM1 | Forkhead box M1 | 1.53 | 0.0024 |

TABLE 5

| Genbank | Gene Symbol | Gene Name | Fold Change | P-value |
|---|---|---|---|---|
| NM_001005340 | GPNMB | Glycoprotein (transmemberane) nmb | −2.40 | 4.0E−04 |
| NM_006216 | SERPINE2 | Serpin peptidase inhibitor, clade E, member 2 | −2.13 | 9.3E−05 |
| NM_054110 | GALNTL2 | UDP-N-acetyl-alpha-D- | −2.11 | 0.00058 |
| | | galactosamine:polypeptide | | |
| | | N-acetylgalactosaminyltransferase-like 2 | | |
| ENST00000356108 | ZNF578 | Zinc finger protein 578 | −1.99 | 0.00046 |
| NM_002153 | HSD17B2 | Hydroxysteroid (17-beta) dehydrogenase 2 | −1.96 | 4.2E−05 |
| NM_002353 | TACSTD2 | Tumor-associated calcium signal transducer 2 | −1.90 | 0.00018 |
| ENST00000282869 | ZNF117 | Zinc finger protein 117 | −1.78 | 0.00022 |
| NM_017762 | MTMR10 | Myotubularin related protein 10 | −1.75 | 4.80E−05 |
| NM_177531 | PKHD1L1 | Polycystic kidney and hepatic disease 1- | −1.69 | 0.00054 |
| | | like 1 | | |
| NM_025208 | PDGFD | Platelet-derived growth factor D | −1.69 | 2.0E−04 |
| NM_004694 | SLC16A6 | Solute carrier family 16, member 6 | −1.68 | 0.00016 |
| NM_004065 | CDR1 | Cerebellar degeneration protein-1 | −1.65 | 1.0E_04 |
| NM_145176 | SLC2A12 | Solute carrier family 2, member 12 | −1.64 | 0.00037 |
| NM_006528 | TFPI2 | Tissue factor pathway inhibitor 2 | −1.60 | 0.00047 |
| NM_021229 | NTN4 | Netrin 4 | −1.60 | 0.00057 |
| NM_003692 | TMEFF1 | Transmembrane protein with EGF-like and two | −1.60 | 0.00062 |
| | | follistatin-like domains 1 | | |
| NM_199355 | ADAMTS18 | ADAM metallopeptidase with thrombospondin | −1.58 | 2.0E−04 |
| | | type 1 motif, 18 | | |
| NM_015881 | DKK3 | Dickkopf homolog 3 | −1.58 | 0.00013 |
| NM_015589 | SAMD4A | Sterile alpha motif domain containing 4A | −1.58 | 0.00011 |
| NM_014271 | IL1RAPL1 | Interleukin 1 receptor accessory protein- | −1.58 | 0.00031 |
| | | like 1 | | |
| NM_001554 | CYR61 | Cysteine-rich, angiogenic inducer, 61 | −1.57 | 0.00015 |
| NM_016205 | PDGFC | Platelet-derived growth factor C | −1.57 | 3.0E−04 |
| NM_021244 | RRAGD | Ras-related GTP binding D | −1.53 | 0.00023 |
| NM_001257 | CDH13 | Cadherin 13, H-cadherin (heart) | −1.52 | 0.00048 |

TABLE 6

| GO Term | Genes |
|---|---|
| Cell cycle | ANLN, ASPM, BUB1, BUB1B, CCNB2, CDC20, |
| GO: 0007049, p = 1.41E−15 | CDC25C, CDKN3, CENPH, CEP55, CIT, DAZL, |
| | DLG7, KIF11, MKI67, MPHOSPH1, NCAPH, NEK2, |
| | NUF2, NUSAP1, PDGFC, PDGFD, PRC1, PTTG1, |
| | RACGAP1 |
| Microtubule cytoskeleton | ASPM, KIF2C, KIF11, KIF14, KIF15, KIF18A, |
| GO: 0015630, p = 3.2E−05 | KIF20A, MPHOSPH1, NEK2, NUSAP1, PRC1, SPRY2 |
| Cytoskeleton | ASPM, CIT, ELMOD1, KIF2C, KIF11, KIF14, |
| GO: 0005856, p = 0.0018 | KIF15, KIF18A, KIF20A, MPHOSPH1, NEK2, PRC1, |
| | SPRR2E, SPRY2 |
| PDGF receptor binding | PDGFC, PDGFD |
| GO: 0005161, p = 0.00183 | |
| Vascular development | BMP4, CYR61, DLL4, EFNB2, EGFL7, FOXM1, |
| GO: 0001944, p = 0.00462 | GJA5, TGFBR1 |

miR-126 Regulates EGFL7 Expression in a Negative Feed-Back Loop

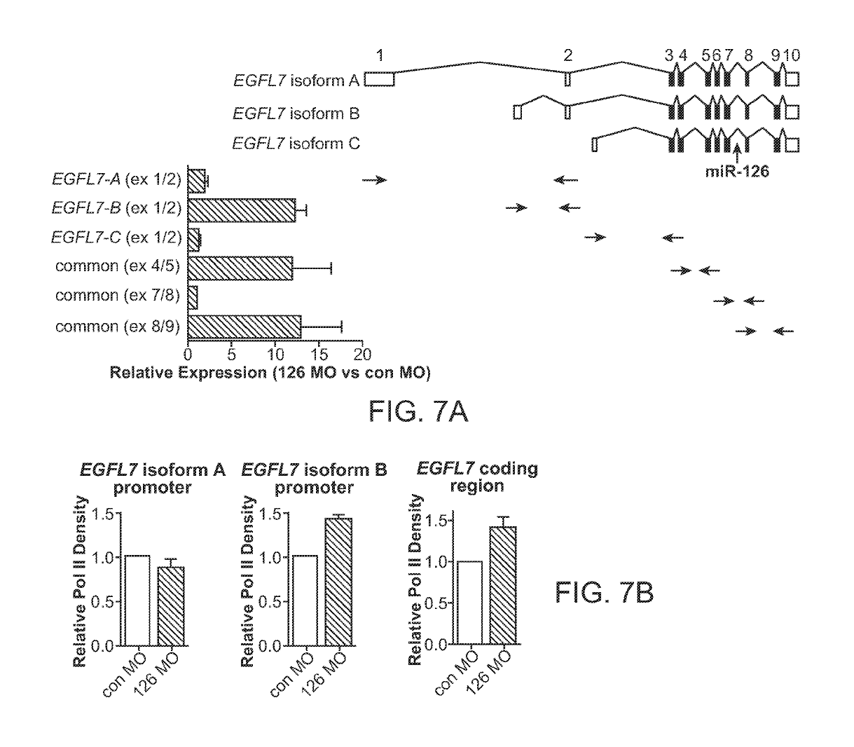

EGFL7 mRNA was highly upregulated on the human array despite our previous finding that levels of spliced EGFL7 mRNA and protein were unchanged. To understand this discrepancy, qRT-PCR with primer sets specific for the transcriptional start sites of the three EGFL7 isoforms (named here EGFL7 isoform-A, -B and -C, which all contain the same open reading frame (ORF)), as well as several primer sets that were common to all three isoforms, was used. EGFL7 mRNA levels were increased throughout the EGFL7 transcriptional unit (FIG. 7A), except for the spliced EGFL7 mRNA surrounding the miR-126-containing intron, as noted earlier (FIG. 3B). Thus, EGFL7 is upregulated in miR-126 MO-treated cells, but the MO apparently inhibits processing of the intron containing miR-126, resulting in no net change in EGFL7 protein levels. Only the EGFL7 isoform B was induced by miR-126 MO (FIG. 7A). Since all three isoforms contain the same 3′ UTR, miR-126 may regulate one of the isoforms in a 3′ UTR-independent fashion. By performing RNA polymerase II (Pol II) chromatin immunoprecipitation (ChIP) experiments an increase in Pol II density at the promoter of isoform B and in the coding region (which is common to all three isoforms), but not at the promoter of isoform A, which was not induced by miR-126 MO (FIG. 7B), was noted. Thus, miR-126 may regulate EGFL7 isoform B at the transcriptional level.

miR-126 Represses SPRED1, VCAM1 and PIK3R2 Post-Transcriptionally

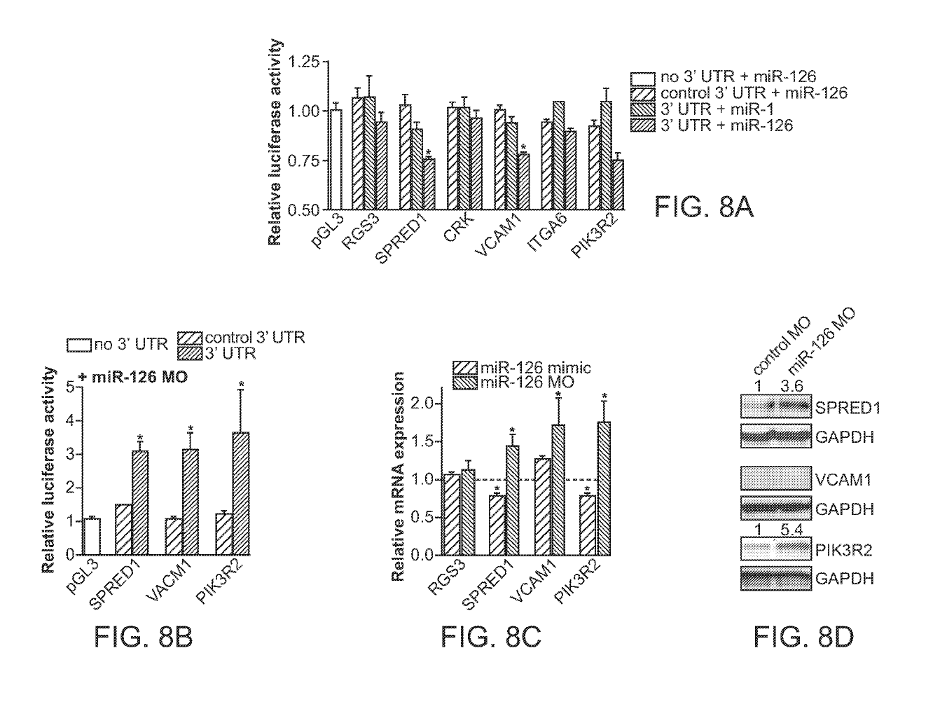

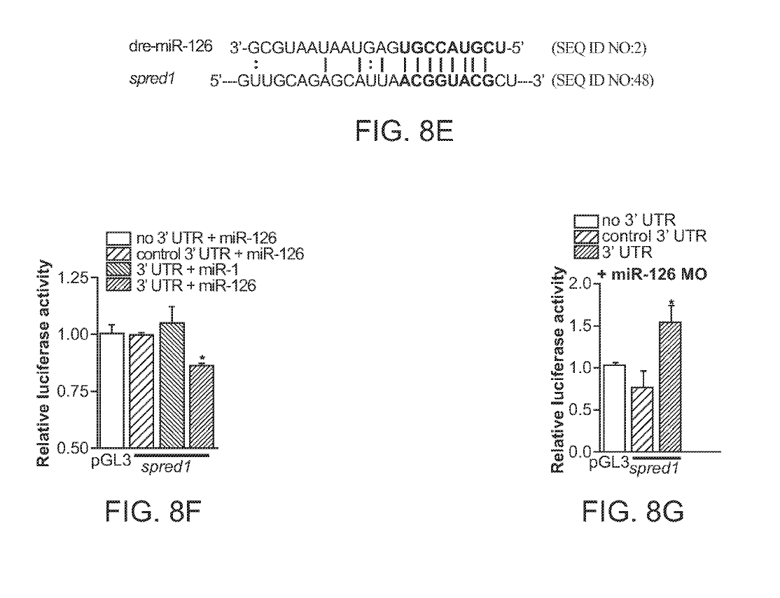

To understand the mechanisms by which miR-126 regulates endothelial biology a search for potential mRNA targets of miR-126 was conducted. Several miRNA target prediction algorithms were employed, including one developed in this laboratory, that incorporates sequence complementarity and mRNA target site accessibility. Portions of the 3′ UTR of several potential targets were cloned into the 3′ UTR of a luciferase construct, and the ability of miR-126 to affect luciferase expression was determined in HeLa cells, which do not normally express miR-126. Six potential targets were initially chosen based on binding sites (FIG. 6B) and a known role in cell signaling or vascular function. These included: regulator of G-protein signaling 3 (RGS3) (Bowman et al. (1998) J. Biol. Chem. 273:28040; Lu et al. (2001) Cell 105:69); SPRED1 (Wakioka et al. (2001) Nature 412:647); PIK3R2 (also known as p85-β) (Ueki et al. (2003) J. Biol. Chem. 278:48453); CRK (Park et al. (2006) Mol. Cell. Biol. 26:6272); integrin alpha-6 (ITGA6); and vascular cell adhesion molecule 1 (VCAM1). miR-126, but not a control miRNA, miR-1, significantly repressed the activity of luciferase derived from RNAs containing the 3′ UTR of SPRED1, VCAM1, and PIK3R2 (FIG. 8A).

Luciferase experiments were also performed in endothelial cells in which endogenous miR-126 levels were knocked down by antisense MO. The activity of luciferase from constructs that included portions of the SPRED1, VCAM1 or PIK3R23′ UTR was increased upon knockdown of miR-126 (FIG. 8B). In contrast, a MO directed to miR-21, which is also expressed in endothelial cells, had no effect on the activity of the constructs tested.

MicroRNAs can regulate mRNA stability or translation of target mRNAs. mRNA expression of potential miR-126 targets was quantified by qRT-PCR in HUVECs that had been transfected with antisense miR-126 MO or a miR-126 mimic (FIG. 8C). While SPRED1 and PIK3R2 mRNA levels were reciprocally regulated by miR-126 abundance, VCAM1 mRNA levels were elevated upon miR-126 inhibition, but were not decreased in the presence of miR-126 mimic. As a control, levels of RGS3 were examined, since the 3′ UTR of this gene did not affect luciferase activity in the presence of miR-126. RGS3 expression was unchanged when miR-126 levels were modulated (FIG. 8C). Expression of SPRED1, PIK3R2 and VCAM1 protein was also assessed by western blot after introduction of control or miR-126 MOs (FIG. 8D). SPRED1 and PIK3R2 protein were increased when miR-126 levels were decreased (FIG. 8D). However, VCAM1 protein was not detected in either control or miR-126 MO-transfected endothelial cells. This was not due to an ineffective antibody, since VCAM1 protein was readily detectible in TNF-α-treated endothelial cells. Indeed, VCAM1 has recently been identified as a miR-126 target in TNF-α-treated endothelial cells (Harris et al. (2008) Proc. Natl. Acad. Sci. USA 105:1516).

The 3′ UTR of zebrafish spred1 contains a highly conserved 8-mer that is perfectly complementary to nucleotides 2-9 of miR-126 (FIG. 8E). Addition of miR-126 specifically repressed the activity of luciferase reporters containing this 3′ UTR (FIG. 8F). Conversely, knockdown of miR-126 led to an increase in luciferase activity of the spred1 3′ UTR luciferase construct when transfected into HUVECs (FIG. 8G). This suggests that miR-126 targeting of SPRED1 is conserved in zebrafish.

FIGS. 7A and 7B. A feed-back loop involving miR-126 regulates EGFL7 expression. (A) Schematic of the A, B, and C isoforms of EGFL7, which initiate from separate promoters, but contain the same open reading frame (ORF). Exons are indicated by numbered boxes, with the ORF indicated by solid boxes. qRT-PCR was performed in endothelial cells treated with miR-126 MO using primers (head-to-head arrows) specific to the three isoforms, as well as common to all three isoforms. The common regions assessed were exon 4/5, exon 7/8, and exon 8/9. The exon 7/8 primer-set spanned the intron containing miR-126. Shown is the fold change in RNA abundance in cells transfected with miR-126 MO. Only EGFL7 isoform B was induced by miR-126 inhibition. (B) RNA Pol II ChIP was performed in miR-126 MO-treated cells for the promoter of EGFL7 isoforms A and B, and at a common coding region (exon 3/intron 3). Isoform B appeared to be transcriptionally induced by miR-126 inhibition.

FIGS. 8A-G. Identification of miR-126 mRNA targets. (A) Relative luciferase activity of constructs containing the 3′ UTR of potential miR-126 targets introduced into HeLa cells in the presence of miR-1 or miR-126. The 3′ UTR was also inserted in the antisense orientation as a control (control 3′ UTR). Firefly luciferase activity for each construct was normalized to the co-transfected Renilla luciferase construct and then normalized to the change in pGL3 luciferase in the presence of microRNA. For each construct, normalized luciferase activity in the absence of microRNA was set to 1. *p<0.05 compared to pGL3. (B) Relative luciferase activity of select constructs in (A) in HUVECs upon inhibition of miR-126 with MOs. (C) mRNA levels of potential targets in HUVEC cells transfected with miR-126 mimic or MO quantified by qRT-PCR. Values are relative to transfection controls. (D) Immunoblot of SPRED1, VCAM1 and PIK3R2 protein in control or miR-126 MO-transfected HUVECs. GAPDH is shown as a loading control. (E) Sequence complementarity of a potential miR-126 binding site in the zebrafish spred1 3′ UTR. (F and G) Luciferase assays (as in (A) and (B), respectively) using the zebrafish spred1 3′ UTR.

miR-126 Modulates VEGF-Dependent Events by Targeting SPRED1 and PIK3R2

SPRED1 and PIK3R2 negatively regulate growth factor signaling via independent mechanisms. SPRED1 functions by inhibiting VEGF-induced activation of the MAP kinase pathway (Taniguchi et al. (2007) Mol. Cell. Biol. 27:4541), while PIK3R2 is thought to negatively regulate the activity of PI3 kinase (Ueki et al. (2003), supra). Activation of the MAP and PI3 kinase pathways by VEGF stimulation can be assessed by measuring the phosphorylation status of ERK and AKT, downstream targets of these pathways, respectively. It was found that the VEGF-induced phosphorylation of ERK and AKT were lower in miR-126 knockdown cells (FIG. 9A). This corresponded with increased SPRED1 and PI3KR2 protein levels. The defect in VEGF-dependent AKT phosphorylation was rescued by siRNA-mediated knockdown of PI3KR2 in miR-126 deficient cells (FIG. 9B). Similarly, inhibition of SPRED1 in cells transfected with miR-126 morpholinos rescued the defect in ERK phosphorylation (FIG. 9C). Experiments were also conducted to determine if decreasing SPRED1 expression in miR-126 knockdown cells could rescue the VEGF-dependent migration defect described earlier (FIG. 9C). While SPRED1 MO alone had no effect on VEGF-induced endothelial cell migration, the knockdown of SPRED1 protein largely rescued the migration defect in cells with decreased miR-126 expression (FIG. 9D).

FIGS. 9A-D. miR-126 positively regulates VEGF signaling in endothelial cells by repressing SPRED1 and PIK3R2. (A) Immunoblot of lysates from HUVECs transfected with control or miR-126 MOs in the presence or absence of VEGF. VEGF induced phosphorylation of ERK (p-ERK) and AKT (p-AKT), which was blocked by miR-126 inhibition. Total ERK and AKT were not affected. Densitometric analysis of normalized protein levels are indicated above. (B) PIK3R2 mRNA was knocked-down by RNAi in HUVECs transfected with control or miR-126 MOs (qRT-PCR) Immunoblot indicates a decrease in PIK3R2 protein by introduction of siRNA, even in the presence of miR-126 MOs. Knockdown of PIK3R2 rescued the defect in VEGF-dependent phosphorylation of AKT in miR-126 MO-treated cells. (C) Immunoblot shows reduction of SPRED1 levels by transfection of a MO that blocks SPRED1 translation, even in the presence of miR-126 MO. SPRED1 MO rescued the defect in VEGF-induced phorphorylation of ERK in miR-126 MO-transfected cells. (D) Quantification of percent (%) wound closure of endothelial cells in a “scratch” assay reveals rescue of miR-126 MO effects by knocking down SPRED1. *p<0.05 compared to control MO.

### Increased Spred1 in Zebrafish Causes Vascular Instability and Hemorrhage

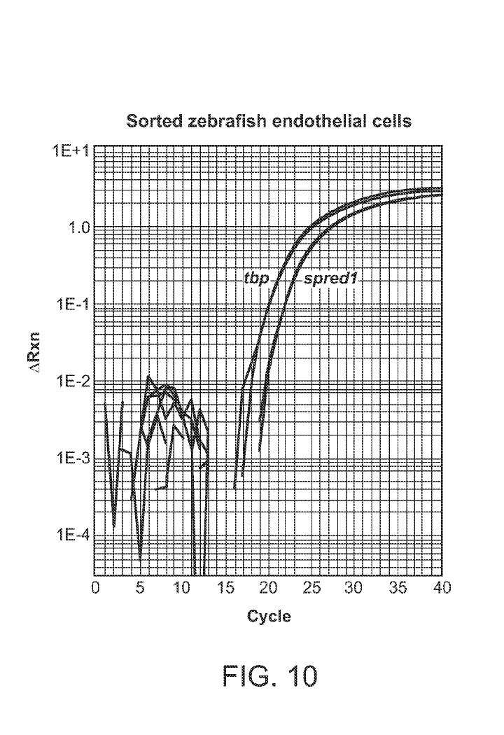

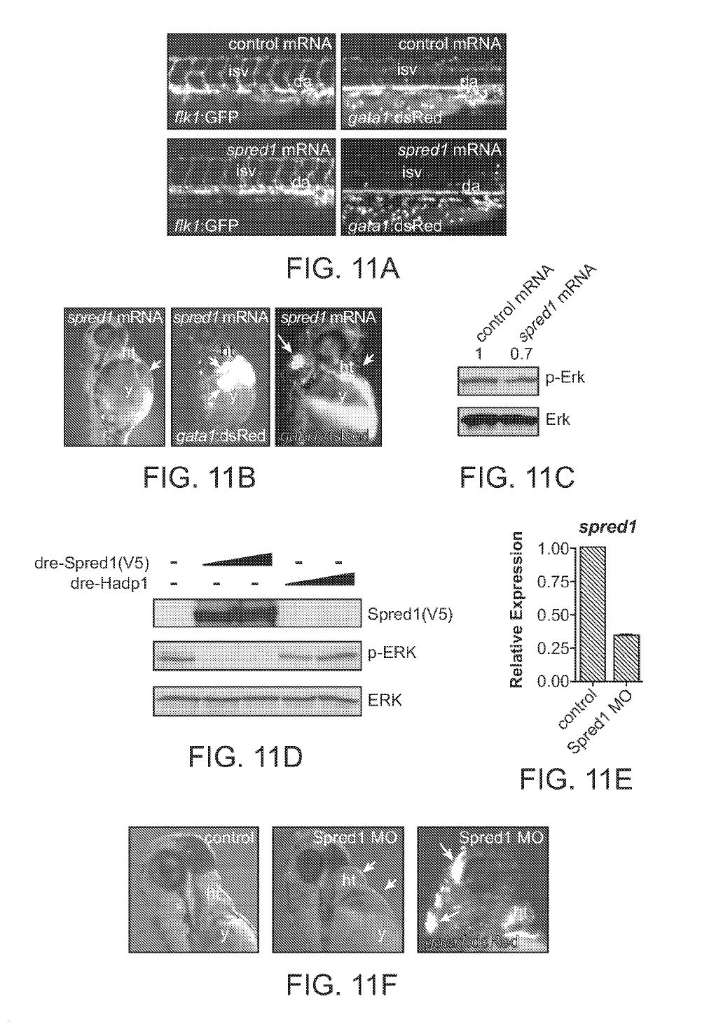

To determine whether excess Spred1 in zebrafish could cause vascular defects similar to miR-126 inhibition, spred1 mRNA, which is normally expressed in endothelial cells (FIG. 10), was injected into zebrafish embryos. Vascular patterning as assessed by Tg(flk1:GFP)^{s843} expression was relatively normal in the majority of spred1 mRNA-injected embryos (FIG. 11A, left panels). However, the presence of blood cells marked by Tg(gata1:dsRed)^{sd2} expression was markedly decreased or absent in the ISVs, DA and PCV (FIG. 11A, right panels). Greater than 30% of the embryos developed cranial and pericardial hemorrhages, indicating the presence of blood cells but the lack of vascular integrity (FIG. 11B). Most of these defects were similar to that observed with miR-126 inhibition and were not prevalent in controls. Consistent with the known function of Spred1, spred1 mRNA-injected embryos had decreased levels of phosphorylated ERK, suggesting diminished growth factor signaling (FIG. 11C). Additionally, transfection of COS-1 cells with an expression construct containing zebrafish spred1 cDNA resulted in dramatically reduced levels of phosphorylated ERK, confirming that zebrafish Spred1, like its mammalian counterpart, negatively regulates the MAP kinase pathway (FIG. 11D). The phenotypic and functional similarities in embryos with increased expression of Spred1 compared to those with increased Spred1 secondary to miR-126 inhibition suggests that Spred1 may be a major mechanism by which miR-126 regulates vascular stability.

Experiments were conducted to test whether knockdown of Spred1 (FIG. 11E) could rescue the vascular defects in miR-126 morphants; however, severe consequences of Spred1 inhibition were observed. The Spred1 MO-injected embryos developed cranial hemorrhages and pericardial edema, even at low MO doses (FIG. 11F). The similarity of this phenotype to the increased expression of Spred1 demonstrates the sensitivity of vascular integrity to Spred1 dosage.

FIG. 10. Spred1 is expressed in zebrafish endothelial cells. spred1 expression was quantified in FACS-isolated GFP⁺ endothelial cells from 72 hpf Tg(flk1:GFP)^{s843} zebrafish embryos by real-time PCR. Shown is the amplification curve for spred1 and the endogenous control gene tbp.

FIGS. 11A-F. Increased Spred1 causes vascular instability and hemorrhage similar to miR-126 knockdown. (A) Lateral view of trunk region of 48 hpf embryos after injection of control (hadp1) or 100 pg of spred1 mRNA. flk1:GFP reveals normal vascular patterning but gata1:dsRed shows diminished blood cells in the dorsal aorta (da) and intersomitic vessels (isv). (B) Injection of spred1 mRNA also resulted in pericardial (left panels; arrowheads) and cranial (right panel; arrow) hemorrhage visualized by gata1:dsRed marking of blood cells. (C) Immunoblot shows a decrease in phosphorylated Erk in 8 hpf zebrafish injected with spred1 mRNA but not with control mRNA (hadp1). Densitometric analysis is shown above. (D) Zebrafish Spred1, but not Hadp1, expression also reduced p-ERK in COS cells as observed by immunoblot. (E) spred1 expression was inhibited by injection of a MO that was designed to generate a splicing product that results in a premature stop codon and nonsense-mediated decay of spred1. Shown is spred1 expression by qRT-PCR in 72 hpf embryos. (F) Lateral view of 72 hpf zebrafish embryos injected with Spred1 MO showing pericardial edema (middle panel; arrowheads) and cranial hemorrhage (right panel; arrows).

A miR-126 Antagomir Reduces Angiogenesis In Vivo.

A miR-126 antagomir of the sequence 5′-cgcauuauuacucacgguacga-3′ (SEQ ID NO:3) was synthesized. All of the nucleotides of the miR-126 antagomir include a 2′-OMe modification. The miR-126 antagomir also included two phosphorothioate linkages at the 5′ end and four phosphorothioate linkages at the 3′ end. In addition, a cholesterol moiety was covalently linked to the 3′ end of the miR-126 antagomir.

RIP-Tag mice express SV40 large T antigen, where the SV40 large T antigen-encoding nucleotide sequence is under the control of a rat insulin promoter, in β islet cells of the pancreas. RIP-Tag mice develop hyperplastic and dysplastic islets that eventually become angiogenic, form invasive carcinomas, and metastasize. Hanahan et al. (1985) Nature 315:115.

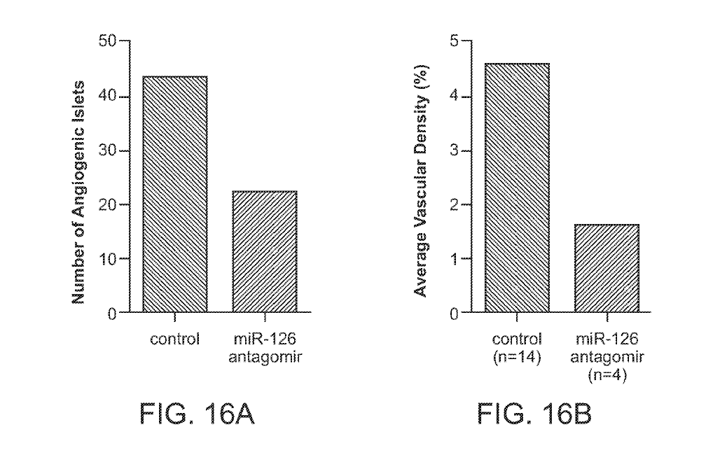

6 week old RIP-Tag mice were treated with a single injection of phosphate buffered saline (PBS; “control”) or 80 mg/kg of miR-126 antagomir. Pancreati were harvested two weeks after the injection. The number of angiogenic islets, and the average vascular density, were assessed. The data are shown in FIGS. 16A and 16B.

FIGS. 16A and 16B. (A) The number of angiogenic islets in the pancreati was quantified. (B) Vascular density within the angiogenic islets was calculated by Metamorph analysis of FITC-lectin-positive cells. The data were averaged based on the number of angiogenic islets measured.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A composition comprising an antisense nucleic acid comprising a nucleotide sequence that is complementary to a nucleotide sequence included in a miR-126 nucleic acid, wherein the antisense nucleic acid comprises a nucleotide sequence capable of forming a stable duplex with a portion of the miR-126 nucleic acid comprising a ribonuclease III cleavage site, and wherein the antisense nucleic acid has a length of from about 20 nucleotides to about 50 nucleotides.

2. The composition of claim 1, wherein the miR-126 nucleic acid comprises a nucleotide sequence having at least about 80% nucleotide sequence identity to nucleotides 15-74 of the nucleotide sequence depicted in FIG. 12A and set forth in SEQ ID NO:1.

3. The composition of claim 1, wherein the antisense nucleic acid comprises fewer than five mismatches in complementarity with the portion of the miR-126 nucleic acid comprising a ribonuclease III cleavage site.

4. The composition of claim 1, wherein the ribonuclease III cleavage site is a Dicer cleavage site or a Drosha cleavage site.

5. (canceled)

6. The composition of claim 1, wherein the antisense nucleic acid comprises at least one non-phosphodiester internucleosidic linkage, at least one modified nucleotide, or at least one substituted sugar moiety.

7.-17. (canceled)

18. A method of reducing angiogenesis in a mammal, the method comprising administering to a mammal in need thereof an effective amount of an agent that decreases a level and/or activity of miR-126 in an endothelial cell in the mammal.

19. The method of claim 18, wherein the agent is: i) an antisense nucleic acid that reduces a level of miR-126 in the cell; ii) a nucleic acid comprising a nucleotide sequence encoding an antisense nucleic acid that reduces a level of miR-126 in the cell; iii) a nucleic acid comprising a nucleotide sequence that is complementary to a mature miR-126 nucleic acid and that inhibits binding of a mature miR-126 to a miR-126 target; or iv) a nucleic acid comprising a nucleotide sequence encoding a nucleic acid comprising a nucleotide sequence that is complementary to a mature miR-126 nucleic acid and that inhibits binding of a mature miR-126 to a miR-126 target.

20. The method of claim 19, wherein the antisense-encoding nucleotide sequence is operably linked to an endothelial-specific transcriptional control element, or to an inducible promoter.

21. (canceled)

22. The method of claim 18, wherein the agent is an antisense nucleic acid that reduces a level of miR-126 in the cell.

23. The method of claim 22, wherein the antisense nucleic acid forms a stable duplex with a portion of the miR-126 nucleic acid comprising a ribonuclease III cleavage site.

24.-26. (canceled)

27. The method of claim 18, wherein the agent is a target protector nucleic acid that binds to a miR-126 target mRNA, and that does not induce cleavage or translational repression of the target mRNA, wherein the target protector nucleic acid inhibits binding of a miR-126 to the miR-126 target mRNA.

28. The method of claim 27, wherein the target mRNA is a Spred1 mRNA or a Pik3r2 mRNA.

29.-32. (canceled)

33. A method of increasing angiogenesis in an individual, the method comprising administering to a mammal having a disorder that is treatable by increasing angiogenesis an effective amount of an agent that increases a level of miR-126 in an endothelial cell in the mammal, wherein said agent is a recombinant nucleic acid comprising a nucleotide sequence encoding a miR-126 nucleic acid.

34. (canceled)

35. The method of claim 33, wherein said miR-126-encoding nucleotide sequence is operably linked to an endothelial cell-specific promoter.

36. (canceled)

37. The method of claim 33, wherein said miR-126 nucleic acid comprises a nucleotide sequence having at least about 75% nucleotide sequence identity to nucleotides 15-41 of the nucleotide sequence depicted in FIG. 12A and set forth in SEQ ID NO:1.

38. The method of claim 33, wherein said administering is via delivery to a local site, or via system administration.

39.-42. (canceled)

43. A synthetic target protector nucleic acid that binds to a miR-126 target mRNA, wherein the target protector nucleic acid does not induce cleavage or translational repression of the target mRNA, and wherein the target protector nucleic acid inhibits binding of a miR-126 to the miR-126 target mRNA.

44. The nucleic acid of claim 43, wherein the target mRNA is a Spred1 mRNA, a Pik3r2 mRNA, a CRK mRNA, an RGS3 mRNA, an IGFA6 mRNA, or a VCAM1 mRNA.

45. The nucleic acid of claim 43, wherein the synthetic target protector nucleic acid comprises a nucleotide sequence having at least about 85% nucleotide sequence identity to the complement of any one of the nucleotide sequences depicted in FIG. 6B.

46. The nucleic acid of claim 43, wherein the target mRNA is Spred1, and wherein the synthetic target protector nucleic acid comprises a nucleotide sequence having at least about 85% nucleotide sequence identity to 5′ TCGTACCTTACATTTAGTTAAA-3′ (SEQ ID NO:32), or wherein the target mRNA is Pik3r2, and wherein the synthetic target protector nucleic acid comprises a nucleotide sequence having at least about 85% nucleotide sequence identity to 5′-ACGTACCGTACAAAACCTGCCT-3′ (SEQ ID NO:33).

47. (canceled)

48. The nucleic acid of claim 43, wherein the synthetic target protector nucleic acid has a length of from about 19 nucleotides to about 50 nucleotides, or from about 19 nucleotides to about 25 nucleotides.

49.-53. (canceled)

54. A composition comprising:
a) the synthetic target protector nucleic acid of claim 43; and
b) a pharmaceutically acceptable carrier.

55. (canceled)

56. A synthetic competitive inhibitor nucleic acid comprising a nucleotide sequence that is complementary to a mature miR-126 nucleic acid and that inhibits binding of a mature miR-126 to a miR-126 target.

57. The synthetic competitive inhibitor nucleic acid of claim 56, wherein the synthetic competitive inhibitor nucleic acid comprises one or more of a nuclease-resistant internucleosidic linkage, a modified nucleotide, and a covalent modification.

58. A recombinant vector comprising a nucleotide sequence encoding the synthetic competitive inhibitor nucleic acid of claim 56.

59. A composition comprising:
a) the synthetic competitive inhibitor nucleic acid of claim 56; and
b) a pharmaceutically acceptable carrier.
